(19) United States
(12) Patent Application Publication
(10) Pub. No.: US 2022/0364119 A1
(43) Pub. Date: Nov. 17, 2022

(54) COMPOSITIONS AND METHODS FOR TREATING AN IMMUNODEFICIENCY VIRUS INFECTION WITH A THERAPEUTIC INTERFERING PARTICLE

(71) Applicant: The J. David Gladstone Institutes, a testamentary trust established under the Will of J.David Gladst, San Francisco, CA (US)

(72) Inventors: Leor S. Weinberger, San Francisco, CA (US); Elizabeth Tanner, San Francisco, CA (US); Seung-Yong Jung, San Francisco, CA (US)

(21) Appl. No.: 17/619,063

(22) PCT Filed: Jun. 12, 2020
(86) PCT No.: PCT/US2020/037488

§ 371 Date: Dec. 14, 2021

Related U.S. Application Data
(60) Provisional application No. 62/861,723, filed on Jun. 14, 2019.

(51) Int. Cl. C12N 15/867; A61K 35/76; A61K 38/16

(52) U.S. Cl. CPC C12N 15/867; A61K 35/76; A61K 38/162; C12N 2740/16032

(57) ABSTRACT

The present disclosure provides interfering, conditionally replicating human immunodeficiency virus (HIV) constructs; infectious particles comprising the constructs; and compositions comprising the constructs or the particles. The constructs, particles, and compositions are useful in methods of reducing HIV viral load in an individual, which methods are also provided.

### PRIORITY APPLICATIONS

This application claims the benefit of priority to U.S. Provisional Patent Application Ser. No. 62/861,723, filed Jun. 14, 2019, the content of which is incorporated herein by reference in its entirety.

### STATEMENT REGARDING FEDERALLY SPONSORED RESEARCH

This invention was made with government support under Grant No. 5DPIDE024408-05 (DP1-R02427) awarded by the National Institutes of Health and Grant No. D17AC00009 (INTERCEPT-R02564) awarded by DOD/DARPA. The government has certain rights in the invention.

### INCORPORATION BY REFERENCE OF SEQUENCE LISTING PROVIDED AS A TEXT FILE

A Sequence Listing is provided herewith as a text file, “2047936.txt” created on Jun. 12, 2020 and having a size of 81,920 bytes. The contents of the text file are incorporated by reference herein in their entirety.

### INTRODUCTION

Despite the transformative success of antiretrovirals, 37 million people are living with HIV/AIDS. Current interventions require high patient engagement but many infected individuals, particularly those in low-resource settings, are unable to access consistent care. In 2003, Weinberger et al. proposed a strategy to reduce the burden on patients: a transmissible antiviral that amplifies in the presence of virus and enters HIV transmission chains, making it likely to encounter high-risk individuals (Weinberger, 2003 & 2011). The concept, called Therapeutic Interfering Particles (TIPs), draws inspiration from defective interfering particles (DIPs), replication-defective viral genomes that hijack replication and/or packaging proteins from wild-type virus, thereby interfering with viral outgrowth (FIG. 1A). Although DIPs were originally proposed as a viral countermeasure back in the 1970s (Huang and Baltimore), they have fallen short of their promise due to a limited therapeutic window. For RNA viruses, DIP replication and transmission require sustained co-infection with wild-type virus, otherwise DIP RNAs are rapidly eliminated through degradation. TIPs, on the other hand, are explicitly engineered for persistence through a combination of high transmission rates (i.e. R₀>1) and integration into host cell genomes (Weinberger, Metzger, Igor). Simulations suggest that a single dose of a TIP that meets these persistence criteria and reduces viral load by one log in patients would significantly reduce the likelihood of a patient's progression to AIDS and their risk of transmitting the infection.

### SUMMARY

The present disclosure provides an interfering, conditionally replicating human immunodeficiency virus (HIV) construct; infectious particles comprising the construct; and compositions comprising the construct or the particle. The constructs, particles, and compositions are useful in methods of reducing HIV viral load in an individual, which methods are also provided.

In one aspect, an interfering, conditionally replicating, and conditionally transmitting, recombinant human immunodeficiency virus (HIV) construct is provided, the construct comprising: a) cis-acting elements comprising a long terminal repeat, a Gag-leader sequence, a Ψ packaging signal, a central polypurine tract (cPPT), a rev response element (RRE) sequence, a polypurine tract (PPT), a major splice donor (MSD), A3, and A7; and b) one or more alterations in an HIV nucleotide sequence, wherein the one or more alterations renders each of Pol, Tat, Vpr, Nef, and Vif nonfunctional such that the construct is incapable of replication and production of virus on its own but requires replication-competent HIV to act as a helper virus.

In certain embodiments, the cis-acting elements further comprise D4, A4, and A5.

In certain embodiments, the long terminal repeat is a 3′ long terminal repeat or a 5′ long terminal repeat.

In certain embodiments, the construct further comprises one or more alterations that renders each of Rev, Vpu, and Env nonfunctional.

In certain embodiments, the genomic RNA (gRNA) encoded by the construct is produced at a higher rate than wild-type HIV gRNA when present in a host cell infected with a wild-type HIV, such that the ratio of the gRNA encoded by the construct to the wild-type HIV gRNA is higher than about 1 in the cell.

In certain embodiments, the construct has a higher transmission frequency than the wild-type HIV.

In certain embodiments, the construct has a basic reproductive ratio (R0)>1.

In certain embodiments, the construct does not include any heterologous nucleotide sequences that encode a gene product.

In certain embodiments, the construct is packaged with a higher efficiency than wild-type HIV when present in a host cell infected with a wild-type HIV.

In certain embodiments, the cis-acting elements include at least one cis element embedded within an HIV protein-coding sequence.

In certain embodiments, the construct further comprises one or more alterations comprising a deletion or mutation in an HIV splice donor or acceptor site. For example, one or more of the D2 and D3 splice donor sites and A1 and A2 splice acceptor sites may be deleted. In some embodiments, the D2 and D3 splice donor sites and the A1 and A2 splice acceptor sites are all deleted.

In certain embodiments, one or more alterations in the HIV nucleotide sequence comprise a deletion or a mutation of one or more nucleotides in the nucleotide sequence encoding HIV Pol, a deletion or a mutation of one or more nucleotides in the nucleotide sequence encoding HIV Vif, a deletion or a mutation of one or more nucleotides in the nucleotide sequence encoding HIV Vpr, a deletion or a mutation of one or more nucleotides in the nucleotide sequence encoding HIV Tat, and a deletion or a mutation of one or more nucleotides in the nucleotide sequence encoding HIV Nef.

In certain embodiments, the construct comprises a deletion of the nucleotides corresponding to positions 3159 to 5737, 3159 to 4780, 4904 to 5737, and 8786 to 9048 numbered relative to the reference HIV sequence of SEQ ID NO:1. In certain embodiments, the construct comprises the nucleotides corresponding to positions 1 to 634, 635 to 789, 686 to 823, 790 to 2292, 2085 to 3158, 4781 to 4903, 5738 to 5849, 5830 to 6044, 5872 to 5880, 5969 to 6044, 6045 to 6060, 6061 to 6306, 6221 to 8785, 7759 to 7992, 8369 to 8414, 8369 to 8643, and 9049 to 9709 numbered relative to the reference HIV sequence of SEQ ID NO:1. In certain embodiments, the construct comprises the nucleotide sequence of SEQ ID NO:2, or a nucleotide sequence displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto.

In certain embodiments, the construct further comprises a deletion or a mutation of one or more nucleotides in the nucleotide sequence encoding HIV Rev, a deletion or a mutation of one or more nucleotides in the nucleotide sequence encoding HIV Vpu, and a deletion or a mutation of one or more nucleotides in the nucleotide sequence encoding HIV Env. In certain embodiments, the construct comprises a deletion of the nucleotides corresponding to positions 3159 to 4780, 4904 to 5737, 5850 to 6341, and 8786 to 9048 numbered relative to the reference HIV sequence of SEQ ID NO:1. In certain embodiments, the construct comprises the nucleotides corresponding to positions 1 to 634, 635 to 789, 686 to 823, 790 to 2292, 2085 to 3158, 4781 to 4903, 5738 to 5849, 6342 to 8785, 7759 to 7992, 8369 to 8414, 8369 to 8643, and 9049 to 9709 numbered relative to the reference HIV sequence of SEQ ID NO:1. In certain embodiments, the construct comprises or consists of the nucleotide sequence of SEQ ID NO:3, or a nucleotide sequence displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto.

In certain embodiments, the construct comprises a deletion of the nucleotides corresponding to positions 3159 to 5630 and 8786 to 9048 numbered relative to the reference HIV sequence of SEQ ID NO:1. In certain embodiments, the construct comprises the nucleotides corresponding to positions 1 to 634, 635 to 789, 686 to 823, 790 to 2292, 2085 to 3158, 5631 to 5849, 4778 to 4898, 5850 to 6044, 5969 to 6044, 6045 to 6060, 6061 to 6306, 6221 to 8785, 7759 to 7992, 8369 to 8414, 8369 to 8643, 9049 to 9709 numbered relative to the reference HIV sequence of SEQ ID NO:1. In certain embodiments, the construct comprises or consists of the nucleotide sequence of SEQ ID NO:4, or a nucleotide sequence displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto.

In certain embodiments, the construct further comprises a deletion of the nucleotides corresponding to positions 5850 to 6341, numbered relative to the reference HIV sequence of SEQ ID NO:1. In certain embodiments, the construct comprises the nucleotides corresponding to positions 1 to 634, 635 to 789, 686 to 823, 790 to 2292, 2085 to 3158, 5631 to 5849, 4778 to 4898, 6342 to 8785, 7759 to 7992, 8369 to 8414, 8369 to 8643, 9049 to 9709 numbered relative to the reference HIV sequence of SEQ ID NO:1. In certain embodiments, the construct comprises or consists of the nucleotide sequence of SEQ ID NO:5, or a nucleotide sequence displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto.

In certain embodiments, the construct comprises a deletion in the nucleotide sequence encoding HIV Pol, a deletion of the nucleotide sequence encoding HIV Vif, a deletion of the nucleotide sequence encoding HIV Vpr, a deletion of the nucleotide sequence encoding HIV Tat, a deletion of the nucleotide sequence encoding HIV Rev, a deletion of the nucleotide sequence encoding HIV Vpu, a deletion of the nucleotide sequence encoding HIV Env, a deletion of the nucleotide sequence encoding HIV Nef, or a deletion of the nucleotide sequence encoding HIV Gag, or a combination thereof.

In another aspect, a composition is provided comprising a construct described herein. In some embodiments, the composition further comprises a pharmaceutically acceptable excipient. In some embodiments, the composition further comprises another HIV antiviral therapeutic agent.

In another aspect, a method of treating an individual infected with a human immunodeficiency virus is provided, the method comprising administering a therapeutically effective amount of a pharmaceutical composition comprising a construct described herein to the individual. In some embodiments, the method further comprises administering to the individual an effective amount of an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, viral Pol activity, and viral Vif activity.

In another aspect, a method of reducing human immunodeficiency virus viral load in an individual is provided, the method comprising administering to the individual an effective amount of the pharmaceutical composition comprising a construct described herein. In some embodiments, the method further comprises administering to the individual an effective amount of an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, viral Pol activity, and viral Vif activity.

In another aspect, a particle is provided, the particle comprising a construct described herein and a viral envelope protein. In some embodiments, the envelope protein comprises gp120. In other embodiments, the envelope protein is a non-HIV protein.

In another aspect, a composition comprising a particle comprising a construct described herein and a viral envelope protein is provided. In some embodiments, the composition further comprises a pharmaceutically acceptable excipient. In some embodiments, the composition further comprises another HIV antiviral therapeutic agent.

In another aspect, a kit for treating an infection by a human immunodeficiency virus is provided, the kit comprising a container (e.g., syringe) comprising a pharmaceutical composition comprising a construct described herein.

In another aspect, a kit for treating an infection by a human immunodeficiency virus is provided, the kit comprising a container (e.g., syringe) comprising a pharmaceutical composition comprising a particle comprising a construct, described herein, and a viral envelope protein.

In another aspect, a method of treating an individual infected with a human immunodeficiency virus is provided, the method comprising administering a therapeutically effective amount of a pharmaceutical composition comprising a particle described herein to the individual. In some embodiments, the method further comprises administering to the individual an effective amount of an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, viral Pol activity, and viral Vif activity.

In another aspect, a method of reducing human immunodeficiency virus viral load in an individual is provided, the method comprising administering to the individual an effective amount of the pharmaceutical composition comprising a particle described herein. In some embodiments, the method further comprises administering to the individual an effective amount of an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, viral Pol activity, and viral Vif activity.

In certain embodiments, the methods described herein are used to treat an individual who has been diagnosed with an HIV infection or is considered to be at higher risk than the general population of becoming infected with HIV. Such an individual may be further treated with an effective amount of an agent that reactivates latent HIV integrated into the genome of a cell infected with HIV.

In another aspect, an isolated biological fluid comprising a construct, described herein, is provided such as, for example, including without limitation, blood or plasma.

In another aspect, a method of generating a variant interfering, conditionally replicating, human immunodeficiency virus (HIV) construct is provided, the method comprising: a) introducing a construct described herein into a first individual; b) obtaining a biological sample from a second individual to whom the construct has been transmitted from the first individual, wherein the construct present in the second individual is a variant of the construct introduced into the first individual; and c) cloning the variant construct from the second individual.

In another aspect, an isolated cell comprising a construct, described herein, is provided.

In another aspect, a method of generating a particle is provided, the method comprising transfecting a cell infected with a human immunodeficiency virus with a construct described herein and incubating the cell under conditions suitable for packaging the construct in the particle.

### BRIEF DESCRIPTION OF THE DRAWINGS

FIGS. 1A-1B show the concept of HIV Therapeutic Interfering Particles (TIPs). (FIG. 1A) Schematic of the HIV genome which encodes nine genes (Gag, Pol, Vif, Vpu, Vpr, Env, Tat, Rev, Nef) driven by the (weak) LTR promoter. After integration into the genome (DNA at left), Tat transactivates the LTR (arrow) generating HIV RNAs that ultimately produce the packaging proteins that form the virus capsid and HIV RNAs are packaged if the HIV packaging signal (Ψ) is present on the viral genomic RNA (gRNA). (FIG. 1B) HIV TIPs are pared-down versions of the HIV genome re-engineered to carry Ψ, the LTR, and other viral cis elements required for packaging. TIP RNA can thus only be made by cells that also express HIV proteins (e.g., Tat). TIPs are engineered to produce substantially more gRNA copies than HIV in dually infected cells (see preliminary data). With disproportionately more TIP gRNA than HIV gRNA, HIV's packaging materials are mainly wasted enclosing TIPs rather than HIV. TIPs thus lower HIV burst size and convert infected cells from HIV-producing into mostly TIP-producing, thereby lowering HIV set-point viral load (see FIG. 2).

FIGS. 2A-2B show that TIPs would lower patient HIV viral load and are predicted to outperform conventional interventions in sub-Saharan Africa. (FIG. 2A) A mathematical model of patient viral load predicts that TIPs that stoichiometrically outcompete HIV for intracellular packaging resources will interfere and conditionally mobilize to lower HIV set-point viral load, a strong correlate of clinical progression & transmission. (FIG. 2B) Projected impact of TIP intervention on HIV/AIDS in Malawi based on the UNAIDS antenatal-clinic data set. TIP intervention is compared to: (i) introduction of a 30%-protective vaccine (vaccine 1), based on reported protection levels from the RV 144 “Thai” vaccine trial that is rolled out to an optimistic 80% coverage of all individuals; (ii) a potential 50% protective vaccine (vaccine 2) with optimistic 95% coverage, and (iii) an optimistic antiretroviral ‘test-and-treat’ campaign where 75% of all infections are treated with 99.9% efficacy in halting transmission. TIPs, which reduce HIV viral load by only 0.5-1.5 logs, would produce far greater reduction in prevalence due to inherent targeting of high-risk groups. A TIP that reduces HIV set point by 1-log would lead to ˜5% HIV/AIDS prevalence while a 1.5-log decrease achieves <1% prevalence. Importantly, these models do not account for evolution of HIV resistance, thus, vaccine and antiviral projections are optimistic; from (Metzger et al. 2011).

FIGS. 3A-3C show detection of the HIV F1 DIP from long-term reactor culture. (FIG. 3A) Schematic of automated reactor with cell replenishment for long-term HIV culturing (FIG. 3B) Oscillations in infected CEM cells in an HIV-GFP infection (FIG. 3C) Detection of a ˜2.5kb ‘F1’ deletion provirus in surviving cells at day 80.

FIG. 4 shows general method to construct a randomized, barcoded deletion library for TIP screening. Schematic cycle of method for constructing a barcoded TIP candidate library from a molecular clone: a cycle of in vitro retrotransposition [1], exonuclease-mediated excision of the randomly inserted retrotransposon [2], enzymatic chewback to create a deletion (Δ) [3], and barcoding during re-ligation [4]. (Weinberger and Notton, 2017)

FIGS. 5A-5B show library deletions span HIV's genome. (FIG. 5A) Distribution of transposon insertion sites across the HIV molecular clone, pNL4-3 (from ˜10⁴ colonies). (FIG. 5b) Underlying pNL4-3 map annotated with selected cis (RRE, LTR) and trans elements (gag, pol, env). A 15% subset of the 120,000 variant library is shown. Note absence of deletions in ori/β-lac, which is required for maintaining the pNL4-3 plasmid in E. coli; from (Weinberger and Notton, 2017).

FIGS. 6A-6C show identification of cis-acting elements in HIV from the random-deletion library. (FIG. 6A) High-MOI passage scheme. A diverse library of HIV deletion mutants (light gray virions) are passaged in a permissive T cell line (MT-4) in the presence of NL43 HIV (dark gray virions); only deletion mutants retaining all necessary cis-acting elements are mobilized by HIV to subsequent generations of target cells. (FIG. 6B) After 12 passages in MT-4 (25k mutants), the genotypes of the surviving mutants are evaluated by deep sequencing of the barcode tags and deletion genotypes mapped to the HIV genome (shown in FIG. 6C). Regions of low-deletion depth indicate the presence of cis-acting elements (5′ LTR, 5′ gag, cPPT, RRE, 3′ LTR) intolerant to deletion, whereas regions with high deletion depth are tolerant of deletion (trans-acting elements) and can be deleted to generate a TIP.

FIG. 7 shows that HIV splicing is post-transcriptional. Single-molecule RNA florescence in situ hybridization (FISH) of HIV un-, singly, and multiply spliced transcripts (US, SS, MS) after latency reversal (TNF) followed by actinomycin D (ActD) to halt transcription; from (Hansen et al. 2018).

FIGS. 8A-8D show that the F1^{cPPT} recombinant interferes with HIV. (FIG. 8A) Schematics of NL43 HIV-BFP (reporter in nef) and the F1^{cPPT} encoding a GFP reporter in nef. (FIG. 8B) Two-color flow cytometry of MT-4 cells transduced with F1 (TIP+ cells) or a sham (naïve cells), and then infected with HIV-BFP. Data shown for 1^{st} & 2^{nd} rounds of infection (2 & 4 dpi). F1 interference with HIV evident during 2^{nd} round of infection (reduced BFP on 4 dpi). (FIG. 8C) HIV titer (outgrowth) after viral supernatant transfer from naïve or F1+ cells on 2 dpi to naïve MT-4 cells. Prototype TIP significantly reduces HIV titer (as quantified by % BFP+ cells and cytopathic effect). (FIG. 8D) RT-qPCR: supernatant HIV gRNA is reduced by F1 and HIV mobilizes F1 gRNA, which out-competes and out-packages HIV gRNA ˜60:40. See FIGS. **-** for primary cell and humanized mouse data.

FIG. 9 shows that F1^{cPPT} is mobilized by HIV infection and does not self-mobilize. Flow cytometry analysis of MT-4 co-culture assay: naïve mCherry target cells co-cultured with F1^{cPPT} GFP transduced cells or control GFP+ cells in presence or absence of HIV infection.

FIGS. 10A-10B show that F1^{cPPT} exhibits R₀>1 and reduces HIV's R₀. (FIG. 10A) Rate of HIV and TIP spread in spinner flasks seeded with ˜10% F1^{cPPT} P CEM cells (GFP+) and 88% mCherry+ target CEMs and infected with HIV-BFP at low MOI. % BFP+ and GFP+/mCherry+ double-positive cells were monitored by flow cytometry. Cells were diluted 1:4 when they reached 2 million cells/mL. (FIG. 10B) Calculated R₀ of HIV and F1^{cPPT} TIP from single-round spread. Top, spread of HIV and TIP into mCherry-labeled cells. Bottom, table of R₀ values.

FIG. 11 shows that F1^{cPPT} T stably interferes with HIV spread in long-term culture. Flow cytometry analysis of reactor culture of HIV in CEM cells (black) or HIV in 50% F1^{cPPT} transduced CEM cells (green). Note the putative co-evolutionary ‘blip’ at day 25-35.

FIGS. 12A-12C show that the F1 deletion encompasses RT but RT activity alone is insufficient to explain F1 phenotype. (FIG. 12A) Schematic of F1 deletion. (FIG. 12B) RT enzymatic activity in virions (FIG. 12C) HIV infectious titers as determined by BFP expression in susceptible cells.

FIGS. 13A-13C show that the F1 deletion disrupts protease cleavage of Gag. (FIG. 13A) Schematic of Gag processing (FIG. 13B) FLAQ assay for p24 in viral supernatant (left) and RT-qPCR with primers specific to either HIV or F1^{cPPT} gRNA normalized to p24 (right). (FIG. 13C) Western blot of 293T cells transfected with indicated ratios of HIV or F1 assayed by anti-p24 antibody.

FIGS. 14A-14B show that F1's truncated GagPol protein is packaged into virion particles. (FIG. 14A) Schematic of wild-type HIV and F1 gag-pol coding regions with Gag-Pol protein size indicated. The Gag-Pol protein is made due to a frameshift signal within the gag sequence. (FIG. 14B) Western blot analysis of purified virion particles. 293T cells were transfected with full-length HIV or F1 constructs encoding wild-type protease or a protease inactivating mutation (D25A) to detect uncleaved Gag and Gag-Pol proteins to be detected in virions. At two days post transfection, supernatants were purified and concentrated through a sucrose gradient and virion proteins were analyzed via LICOR™ Western blot using an anti-gag (p24) antibody.

FIGS. 15A-15D show stages of HIV-1 Virion Morphogenesis. Thin section EM images showing: (FIG. 15A) a mature, infectious HIV-1 virion, (FIG. 15B) virions assembling in a cell lacking AMOT, arrested prior to membrane envelopment, (FIG. 15C) virions assembling in a cell lacking TSG101, arrested prior to budding, and (FIG. 15D) virions released from a cell lacking NHP2L1, arrested prior to maturation.

FIG. 16 shows LC MS/MS mass spectrometry detection of proteins in HIV virions. Virions produced in HEK293T cells were purified by ultracentrifugation of filtered culture supernatant over a 20% sucrose layer. The resulting pellet was digested with trypsin and subjected to LC-MS/MS analysis. HIV proteins in bold.

FIGS. 17A-17B show SVA imaging of RNA packaging. (FIG. 17A) Schematics of constructs. HIV NL43 encodes BSL (green) while F1 will encode MSL (red) (FIG. 17B) Representative SVA TIRF imaging of HIV_{BSL} and HIV_{MSL} in a cell (from Chen et al., 2016). Arrows show homodiploid HIV_{BSL} or HIV_{MSL} virions (2 colors) and a heterodiploid HIV_{BSL}-HIV_{MSL} virion (3 colors, middle arrow).

FIGS. 18A-18B show that F1^{cPPT} interferes with HIV-2 lineage lab strains (SIVmac239). (FIG. 18A) Flow cytometry histograms showing that SIVmac239 infection of CEM×174 cells substantially transactivates F1^{cPPT} TIP. In transduced, uninfected CEM×174 cells, GFP activity is modest (green), while SIV infection substantially transactivates F1^{cPPT} (pink, data from 2 dpi). (FIG. 18B) SIV outgrowth as measured by viral cytopathic effect on CEM×174 cells treated with viral supernatant (from 2 dpi) from either CEM×174 (left) or F1 TIP_{cPPT} transduced CEM×174 cells (right). F1 TIP significantly reduces SIV outgrowth in a single round of infection (error bars show STDEV from biological duplicate experiments).

FIGS. 19A-19D show that F1 TIP inhibits HIV and protects CD4 cells in the hu-PBL mouse model. (FIG. 19A) Experimental timeline for engraftment, infection and analysis of mice. (FIG. 19B) CD4+ T cell concentration in TIP-treated and control mice, with and without HIV infection, at one (left) and two (right) weeks post infection. F1^{splice*} TIP contains a double stop codon early in the F1 p51 coding region. Box and whiskers plots, n=8 for each condition. Stars indicate p values calculated within group (i.e. +/− infection) or between no TIP vs. TIP mice. *p≤0.03, **p≤0.003, Mann-Whitney test. (FIG. 19C) Viral loads in mouse serum collected from infected mice 1 wk post infection measured by ddPCR. Points=individual mice; line=mean. Samples with values below the detection threshold are graphed on the x-axis. **p<0.001, one-way ANOVA followed by Tukey's multiple comparisons test. (FIG. 19D) Frequency of full-length HIV and TIP genomes in mouse serum. WT and TIP genomes were quantified by ddPCR 1 wk post infection. Each bar represents a single HIV-infected mouse.

FIG. 20 shows hu-NSG mice exhibit stable HIV set-point for ˜100 d. Plasma viral RNA (copies/ml) kinetics in hu-HSPC-NSG mice before ART (weeks 0-4), during ART (weeks 4-10, shaded region), and after ART (weeks 10-14). Assay sensitivity is ˜750-800 RNA copies/ml (dotted line), since only 50-100 μl of blood was collected per mouse. The solid circles indicate vRNA levels in untreated mice, whereas, the open circles indicate the levels in ART treated animals. (from Satheesan et al. (2018) J. Virol. 92(7) pii: e02118-17).

### DEFINITIONS

The term “immunodeficiency virus” includes human immunodeficiency virus (HIV), feline immunodeficiency virus, and simian immunodeficiency virus. The term “human immunodeficiency virus” as used herein, refers to human immunodeficiency virus-1 (HIV-1); human immunodeficiency virus-2 (HIV-2); and any of a variety of HIV subtypes and quasispecies.

As referred to herein, a “pseudotype envelope” is an envelope protein other than the one that naturally occurs with the retroviral core virion, which encapsidates the retroviral core virion (resulting in a phenotypically mixed virus).

A “virus” is an infectious agent that consists of protein and nucleic acid, and that uses a host cell's genetic machinery to produce viral products specified by the viral nucleic acid. A “nucleic acid” refers to a polymer of DNA or RNA that is single or double-stranded, linear or circular, and, optionally, contains synthetic, non-natural, or modified nucleotides, which are capable of being incorporated into DNA or RNA polymers. A DNA polynucleotide preferably is comprised of genomic or cDNA sequences.

A “wild-type strain of a virus” is a strain that does not comprise any of the human-made mutations as described herein, i.e., a wild-type virus is any virus that can be isolated from nature (e.g., from a human infected with the virus). A wild-type virus can be cultured in a laboratory, but still, in the absence of any other virus, is capable of producing progeny genomes or virions like those isolated from nature.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of an agent (e.g., a construct, a particle, etc., as described herein) that, when administered to a mammal (e.g., a human) or other subject for treating a disease, is sufficient to effect such treatment for the disease. The “therapeutically effective amount” can vary depending on the compound or the cell, the disease and its severity and the age, weight, etc., of the subject to be treated.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, a “pharmaceutical composition” is meant to encompass a composition suitable for administration to a subject, such as a mammal, e.g., a human. In general a “pharmaceutical composition” is sterile, and is free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal and the like.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an interfering particle” includes a plurality of such particles and reference to “the cis-acting element” includes reference to one or more cis-acting elements and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides interfering, conditionally replicating human immunodeficiency virus (HIV) constructs; infectious particles comprising the constructs; and compositions comprising the constructs or the particles. The constructs, particles, and compositions are useful in methods of reducing HIV viral load in an individual, which methods are also provided.

Interfering Constructs

The present disclosure provides an interfering, conditionally replicating human immunodeficiency virus (HIV) construct. For simplicity, the interfering, conditionally replicating HIV constructs are referred to as “interfering constructs” or “TIPs.” A subject interfering construct is conditionally replicating, e.g., a subject interfering construct, when present in a mammalian host, cannot, in the absence of a wild-type HIV, form infectious particles containing copies of itself. A subject interfering construct can be packaged into an infectious particle in vitro in a laboratory (e.g., in an in vitro cell culture) when the appropriate polypeptides required for packaging are provided. The infectious particle can deliver the interfering construct into a host cell, e.g., an in vivo host cell. Once inside an in vivo host cell (a host cell in a mammalian subject), the interfering construct can integrate into the genome of the host cell, or can remain cytoplasmic. The interfering construct can replicate in the in vivo host cell only in the presence of a wildtype HIV. When an in vivo host cell comprising an interfering construct is infected by a wildtype HIV, the interfering construct replicates (e.g., is transcribed and packaged) substantially more efficiently than the wildtype HIV, thereby outcompeting the wildtype HIV. As a result, the HIV viral load is substantially reduced in the individual.

An interfering construct of the present disclosure can be an RNA construct, or a DNA construct (e.g., a DNA copy of an RNA).

An interfering construct of the present disclosure does not include any heterologous nucleotide sequences, e.g., sequences not derived from HIV. An interfering construct of the present disclosure does not include any heterologous nucleotide sequences that encode a gene product. Gene products include polypeptides and RNA. “Heterologous” refers to a nucleotide sequence that is not normally present in a wild-type HIV in nature.

A subject interfering construct comprises HIV cis-acting elements; and comprises an alteration in the HIV nucleotide sequence such that alteration renders one or more encoded HIV trans-acting polypeptides non-functional. By “non-functional” is meant that the HIV trans-activating polypeptide does not carry out its normal function, due to truncation of or internal deletion within the encoded polypeptide, or due to lack of the polypeptide altogether. “Alteration” of an HIV nucleotide sequence includes: deletion of one or more nucleotides and/or substitution of one or more nucleotides.

In some cases, a subject interfering construct, when present in a host cell (e.g., in a host cell in an individual) that is infected with a wildtype HIV, replicates at a rate that is at least about 10%, at least about 20%, at least about 30%, at least about 40%, at least about 50%, at least about 75%, at least about 2-fold, at least about 2.5-fold, at least about 5-fold, at least about 10-fold, or greater than 10-fold, higher than the rate of replication of the wildtype HIV in a host cell of the same type that does not comprise a subject interfering construct.

In some cases, a subject interfering construct, when present in a host cell (e.g., in a host cell in an individual) that is infected with a wildtype HIV, reduces the amount of wildtype HIV transcripts in the cell by at least about 20%, at least about 30%, at least about 40%, at least about 50%, at least about 60%, at least about 70%, at least about 80%, or at least about 90%, compared to the amount of wildtype HIV transcripts in a host cell that is infected with wildtype HIV, but does not comprise a subject interfering construct.

In some cases, a subject interfering construct, when present in a host cell (e.g., in a host cell in an individual) that is infected with a wildtype HIV, results in production of interfering construct-encoded RNA such that the ratio (by weight, e.g., μg:μg) of interfering construct-encoded RNA to wild-type HIV-encoded RNA in the cytoplasm of the host cell is greater than 1. In some cases, a subject interfering construct, when present in a host cell (e.g., in a host cell in an individual) that is infected with a wildtype HIV, results in production of interfering construct-encoded RNA such that the ratio (by weight, e.g., μg:μg) of interfering construct-encoded RNA to wild-type HIV-encoded RNA in the cytoplasm of the host cell is from at least about 1.5:1 to at least about 10²:1 or greater than 10²:1, e.g., from about 1.5:1 to about 2:1, from about 2:1 to about 5:1, from about 5:1 to about 10:1, from about 10:1 to about 25:1, from about 25:1 to about 50:1, from about 50:1 to about 75:1, from about 75:1 to about 100:1, or greater than 100:1.

In some cases, a subject interfering construct, when present in a host cell (e.g., in a host cell in an individual) that is infected with a wildtype HIV, results in production of interfering construct-encoded RNA such that the ratio (e.g., molar ratio) of interfering construct-encoded RNA to wild-type HIV-encoded RNA in the cytoplasm of the host cell is greater than 1. In some cases, a subject interfering construct, when present in a host cell (e.g., in a host cell in an individual) that is infected with a wildtype HIV, results in production of interfering construct-encoded RNA such that the ratio (e.g., molar ratio) of interfering construct-encoded RNA to wild-type HIV-encoded RNA in the cytoplasm of the host cell is from at least about 1.5:1 to at least about 10²:1 or greater than 10²:1, e.g., from about 1.5:1 to about 2:1, from about 2:1 to about 5:1, from about 5:1 to about 10:1, from about 10:1 to about 25:1, from about 25:1 to about 50:1, from about 50:1 to about 75:1, from about 75:1 to about 100:1, or greater than 100:1.

Any convenient method can be used to measure the ratio of interfering construct-encoded RNA to wild-type HIV-encoded RNA in the cytoplasm of the host cell. Suitable methods can include, for example, measuring transcript number directly via qRT-PCR of both an interfering construct-encoded RNA and a wild-type HIV-encoded RNA; measuring levels of a protein encoded by the interfering construct-encoded RNA and the wild-type HIV-encoded RNA (e.g., via western blot, ELISA, mass spectrometry, etc.); and measuring levels of a detectable label associated with the interfering construct-encoded RNA and the wild-type HIV-encoded RNA (e.g., fluorescence of a fluorescent protein that is encoded by the RNA and is fused to a protein that is translated from the RNA). Such measurements can be performed, for example, after co-transfection, using any convenient cell type.

In some embodiments, the interfering construct-encoded RNA is packaged. In some embodiments, the interfering construct-encoded RNA is unpackaged. In some cases, the interfering construct-encoded RNA includes both packaged and unpackaged RNA.

In some cases, a subject interfering construct, when present in a host cell (e.g., in a host cell in an individual) that is infected with a wildtype HIV, dimerizes with wildtype gRNA HIV genomes. In some cases, a subject interfering construct, when present in a host cell (e.g., in a host cell in an individual) that is infected with a wildtype HIV, dimerizes with a wildtype gRNA HIV genome and inhibits dimerization of wild-type HIV.

Both wildtype HIV and a subject interfering construct can be packaged into an infectious particle by a host cell (e.g., in a host cell in an individual). In some cases, where a host cell comprises both a subject interfering construct and a wildtype HIV, the ratio of interfering construct-containing particles produced by the host cell to wildtype HIV-containing particles is greater than 1. In some cases, where a host cell comprises both a subject interfering construct and a wildtype HIV, the ratio of interfering construct-containing particles produced by the host cell to wildtype HIV-containing particles is from at least about 1.5:1 to at least about 10²:1 or 5 greater than 10²:1, e.g., from about 1.5:1 to about 2:1, from about 2:1 to about 5:1, from about 5:1 to about 10:1, from about 10:1 to about 25:1, from about 25:1 to about 50:1, from about 50:1 to about 75:1, from about 75:1 to about 100:1, or greater than 100:1.

A wildtype HIV genome is approximately 9700 nucleotides in length, e.g., from about 9700 nucleotides to about 9800 nucleotides in length). In contrast, a subject interfering construct has a genome that is some fraction of the total HIV genome length, such as from about 1000 nucleotides (nt) to about 9700 nt, e.g., from about 1000 nt to about 2000 nt, from about 2000 nt to about 3000 nt, from about 3000 nt to about 4000 nt, from about 4000 nt to about 5000 nt, from about 5000 nt to about 6000 nt, from about 6000 nt to about 7000 nt, from about 7000 nt to about 8000 nt, from about 8000 nt to about 9000 nt, or from about 9000 nt to about 9700 nt. In some cases, a subject interfering construct has a length of from about 2500 nucleotides (nt) to about 9500 nt, e.g., from about 2500 nt to about 3500 nt, from about 3000 nt to about 4000 nt, from about 3500 nt to about 4500 nt, from about 4000 nt to about 5000 nt, from about 4500 nt to about 5500 nt, from about 5000 nt to about 6000 nt, from about 5500 nt to about 6500 nt, from about 6000 nt to about 7000 nt, from about 6500 nt to about 7500 nt, from about 7000 nt to about 8000 nt, from about 7500 nt to about 8500 nt, from about 8000 nt to about 9000 nt, or from about 8500 nt to about 9500 nt.

A subject interfering construct can exhibit a basic reproductive ratio (R₀) (also referred to as the “basic reproductive number”) that is greater than 1. R₀ is the number of cases one case generates on average over the course of its infectious period. When R₀ is 1, the infection will be able to spread in a population. Thus, a subject interfering construct has the capacity to spread from one individual to another in a population. In some cases, the subject interfering construct (or a subject interfering particle) has an R₀ from about 2 to about 5, from about 5 to about 7, from about 7 to about 10, from about 10 to about 15, or greater than 15.

Cis-Acting Elements

An interfering construct of the present disclosure comprises lentivirus cis-acting elements. Cis-acting elements include, e.g., a lentiviral Ψ (psi) packaging signal; a lentiviral rev responsive element (rre); a lentiviral long terminal repeat (LTR); and a cis element embedded within an HIV protein-coding sequence. Nucleotide sequences for HIV-1 cis-acting elements are known in the art. See, e.g., the following web site: hiv.lanl.gov.

Lentiviral Ψ packaging signal sequences are known in the art. See, e.g., Lever et al. (1989) J. Virol. 63:4085; and McBride et al. (1998) J. Virol. 71:4544. The Ψ packaging signal has a length of from about 80 nt to about 150 nt; and includes four stem-loop (SL) structures: SL1-SL4. A lentiviral Ψ (psi) packaging signal sequence can have at least about 85%, at least 90%, at least 95%, at least 98%, at least 99%, or 100%, nucleotide sequence identity with any known wild-type Ψ packaging signal sequence.

HIV-1 SL-1 (also known in the art as HIV-1_DIS)
can have the sequence:
(SEQ ID NO: 6)
5′-GGACUCGGCUUGCUGAAGYGCRCWCRGCAAGAGGCGAGRG-3′.
HIV-1 SL2 (also known in the art as HIV-1_SD)
can have the sequence:
(SEQ ID NO: 7)
5′-RGCGACUGGUGAGUACGCH-3′.
HIV-1 SL3 can have the sequence:
(SEQ ID NO: 8)
5′-UGACUAGCGGAGGCUAGAAGGAG-3′.
HIV-1 SL4 can have the sequence:
(SEQ ID NO: 9)
5′-UGGGUGCGAGAGCGUCARUA-3′.

In the above-noted sequences, Y is C or T; W is A or T; R is A or G; and H is A, C, or T.

A lentiviral rev responsive element (rre) lies within about nt 7709-8063 of the HIV-1 genome; and has a length of from about 240 nt to about 355 nt. See, e.g., Cullen et al. (1991) J. Virol. 65: 1053; Cullen et al. (1991) Cell 58: 423-426; and Malim et al. (1989) Nature 338(6212):254-7. A suitable lentiviral rre can comprise a nucleotide sequence having at least about 85%, at least 90%, at least 95%, at least 98%, at least 99%, or 100%, nucleotide sequence identity with any known wild-type HIV-1 rre sequence.

HIV-1 LTR sequences are known in the art. A suitable lentiviral 5′-LTR or 3′-LTR can comprise a nucleotide sequence having at least about 85%, at least 90%, at least 95%, at least 98%, at least 99%, or 100%, nucleotide sequence identity with any known wild-type HIV-1 5′LTR or 3′LTR sequence.

In some embodiments, an interfering, conditionally replicating, human immunodeficiency virus (HIV) construct comprises the cis-acting elements including a long terminal repeat, a Gag-leader sequence, a Ψ packaging signal, a central polypurine tract (cPPT), a rev response element (RRE) sequence, a polypurine tract (PPT), a major splice donor (MSD), and A3, and A7. In some embodiments, the cis-acting elements further comprise D4, A4, and A5. In some embodiments, the construct includes a 3′ long terminal repeat, a 5′ long terminal repeat, or a combination thereof.

In certain embodiments, the construct further comprises one or more alterations comprising a deletion or mutation in an HIV splice donor or acceptor site. For example, one or more of the D2 and D3 splice donor sites and A1 and A2 splice acceptor sites may be deleted. In some embodiments, the D2 and D3 splice donor sites and the A1 and A2 splice acceptor sites are deleted.

In certain embodiments, the construct further comprises one or more alterations that renders each of Rev, Vpu, and Env nonfunctional.

Dimerization Initiation Signal

Lentiviruses are diploid and genomic RNAs (gRNAs) are packaged into virions in pairs, where encapsidation of two copies of RNA is achieved by allowing the gRNAs to dimerize. This gRNA pairing is initiated at a six-nucleotide palindrome termed the dimerization initiation signal (DIS) which is located within stem loop 1 (SL1) of the HIV-1 genome and has the consensus sequence GCGCGC.

An interfering construct of the present disclosure can comprise a wildtype DIS, e.g., having the consensus sequence GCGCGC. In other cases, an interfering construct of the present disclosure comprises a DIS with a single nucleotide mutation (e.g., GCGCGC→GCGAGC). The GCGAGC DIS results in reduced HIV-1 homodimerization.

Trans-Acting Elements

An interfering construct of the present disclosure includes one or more alterations in an HIV nucleotide sequence, wherein the one or more alterations renders each of Pol, Tat, Vpr, Nef, and Vif nonfunctional such that the construct is incapable of replication and production of virus on its own but requires replication-competent HIV to act as a helper virus. A wild-type HIV-1 genome gives rise to three classes of RNA: unspliced RNA; incompletely spliced RNA; and fully spliced RNA.

Unspliced RNA: The unspliced 9-kb primary transcript can be expressed to generate the Gag and Gag-Pol precursor proteins or be packaged into virions to serve as the genomic RNA. Incompletely spliced RNA. These mRNAs use the splice donor site located nearest the 5′ end of the HIV RNA genome in combination with any of the splice acceptors located in the central region of the virus. These RNAs can potentially express Env, Vif, Vpu, Vpr, and the single-exon form of Tat. These heterogeneous mRNAs are 4- to 5-kb long and retain the second intron of HIV.

Fully spliced RNA. These mRNAs have spliced out both introns of HIV and have the potential to express Rev, Nef, and the two-exon form of Tat. These heterogeneous mRNAs do not require the expression of the Rev protein.

In some cases, the alteration in the HIV nucleotide sequence is a deletion of one or more nucleotides in a splice donor and/or a splice acceptor. See, e.g., Schwartz et al. (1990) J. Virol. 64:2519. For example, in some instances, the alteration is a deletion or a substitution of one or more nucleotides in the 5′ major splice donor. Nucleotide sequences of the 5′ major splice donor of HIV are known. See, e.g., Harrison and Lever (1992) J. Virol. 66:4144.

An interfering construct of the present disclosure can in some embodiments include a deletion of one or more nucleotides in one or more splice donor and/or splice acceptor sequences of HIV, such that one or more of Env, Gag, Pol, Tat, Rev, Vpr, Nef, Vif, and Vpu are not produced. An interfering construct of the present disclosure can in some embodiments include a mutation of one or more nucleotides in one or more splice donor and/or splice acceptor sequences of HIV, such that one or more of Env, Gag, Pol, Tat, Rev, Vpr, Nef, Vif, and Vpu are not produced. In some instances, none of Env, Gag, Pol, Tat, Rev, Vpr, Nef, Vif, and Vpu is produced. In some instances, Env, Vif, Vpu, Vpr, and Tat are not produced. In some instances, none of Pol, Tat, Vpr, Nef, and Vif is produced. In some instances, none of Env, Pol, Tat, Rev, Vpr, Nef, Vif, and Vpu is produced.

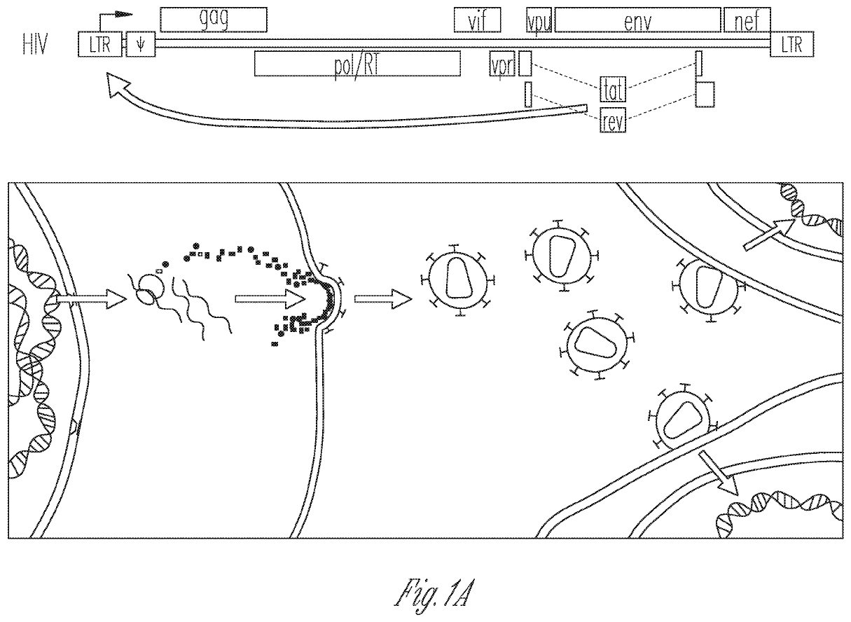

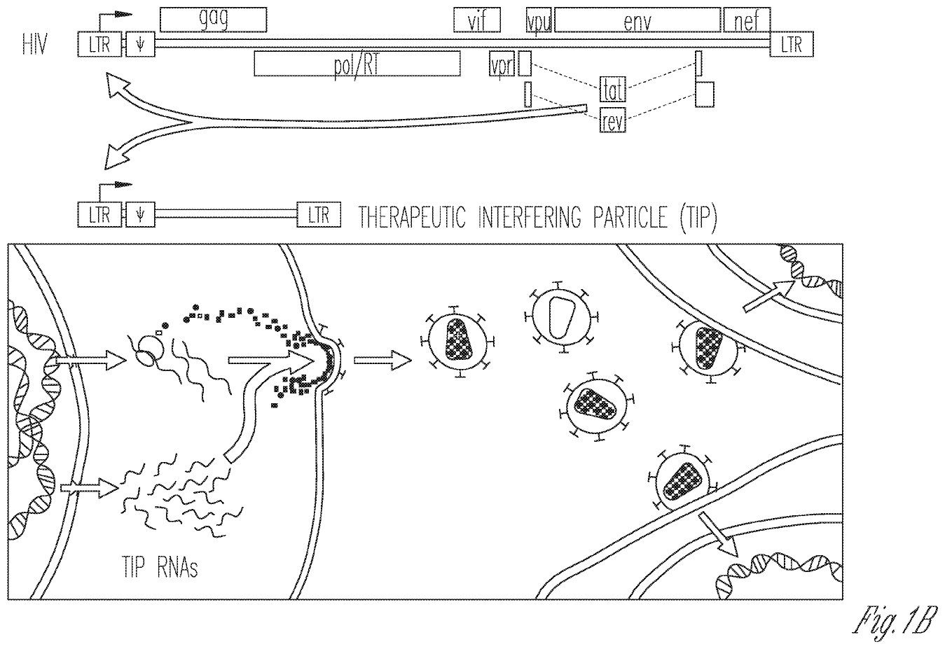

In some instances, an interfering construct of the present disclosure includes a deletion or a substitution of one or more nucleotides in an HIV splice donor selected from D1, D2, D3, and D4. In some instances, an interfering construct of the present disclosure includes a deletion or a substitution of one or more nucleotides in an HIV splice acceptor selected from A1, A2, A3, A4, A5, A6, and A7. See, e.g., FIG. 1 of Mandal et al. ((2010) J. Virol. 84:12790) for the organization of HIV splice donors D1-D4 and splice acceptors A1-A7, relative to locations of exons, in the HIV genome.

In some instances, an interfering construct of the present disclosure includes a deletion or a substitution of one or more nucleotides in the HIV major splice donor, where exemplary wild-type sequences surrounding the major splice donor (D1) include:

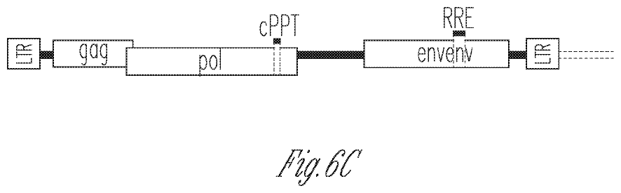

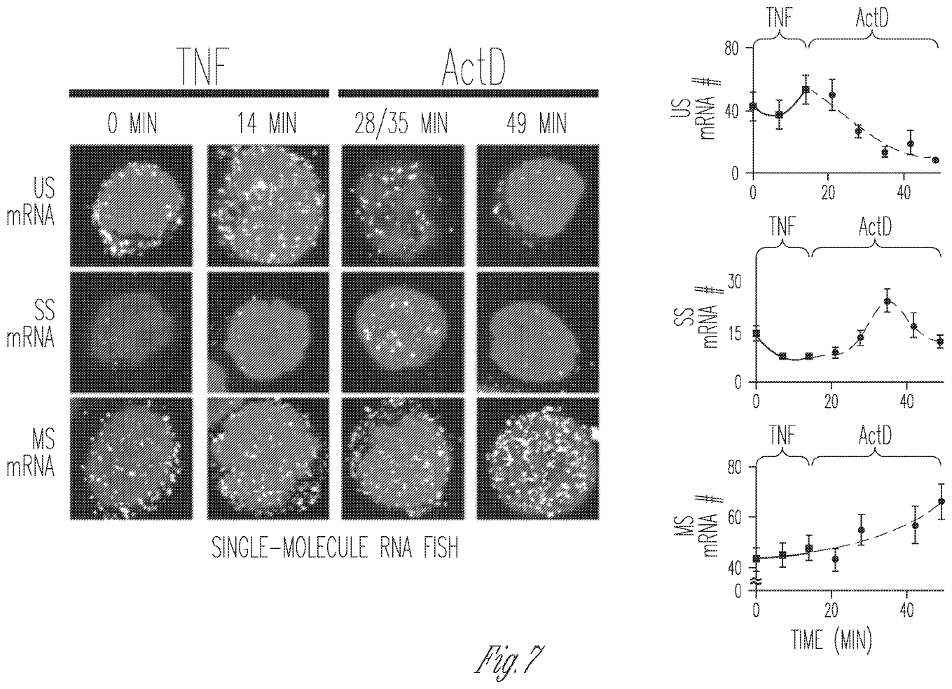

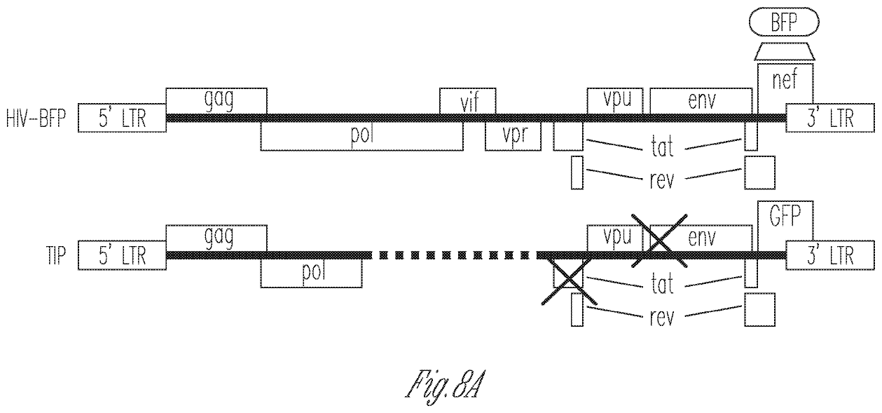

(SEQ ID NO: 10)
5′-ggcgactgGtgagtacgcc-3′;
(SEQ ID NO: 11)
5′-ggcggctgGtgagtacgcc-3′;
(HIVRF)
(SEQ ID NO: 12)
5′-ggcgaatgGtgagtacgcc-3′,
(SEQ ID NO: 13)
5′-agcgactgGtgagtacgct-3′;
and
(HIV2226)
(SEQ ID NO: 14)
5′-agcgaccgGtgagtacgct-3′;

where the “G” in upper case and bold is the major splice donor. See, e.g., FIG. 8 of Harrison and Lever (1992) J. Virol. 66:4144. The major splice donor is approximately 50 nucleotides (e.g., 45 nucleotides to 55 nucleotides) 5′ of the gag initiator ATG.

In some instances, an interfering construct of the present disclosure includes a deletion or a substitution of one or more nucleotides in an HIV splice donor D2. An exemplary nucleotide sequence surrounding HIV splice donor D2 is 5′-AAGGUGAAGGG-3′ (SEQ ID NO:15). In some instances, an interfering construct of the present disclosure includes a deletion or a substitution of one or more nucleotides in an HIV splice donor D3. An exemplary nucleotide sequence surrounding HIV splice donor D3 is 5′-AAGGUAGGUCA-3′ (SEQ ID NO:16). In some instances, an interfering construct of the present disclosure includes a deletion or a substitution of one or more nucleotides in an HIV splice donor D4. An exemplary nucleotide sequence surrounding HIV splice donor D4 is 5′-CUAGACUAGAG-3′ (SEQ ID NO:17). See, e.g., Mandal et al. (2010) J. Virol. 84:12790. Another exemplary nucleotide sequence surrounding HIV splice donor D4 is 5′-GCAGUAAGUAG-3′ (SEQ ID NO:18); see, e.g., Kammler et al. (2006) Retrovirol. 3:89.

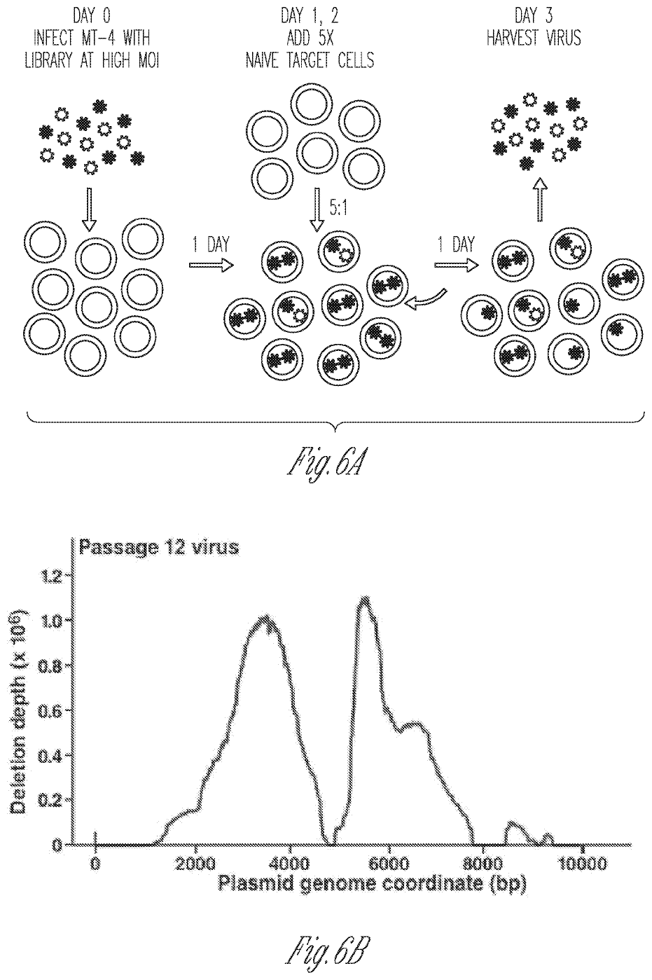

In some instances, an interfering construct of the present disclosure includes a deletion or a substitution of one or more nucleotides in an HIV splice acceptor sequence. Nucleotide sequences surrounding HIV splice acceptor sequences are known in the art. See, e.g., FIG. 7 of Schwartz et al. (1990) J. Virol. 64:2519.

For example, in the sequence: 5′-ataacaaaAGccttAGgcatctcctatggcAGgaagaagagagttAGgcAGggatattcaccattatcgtttcAGcc-3′ (SEQ ID NO:19), the “AG” in upper case and bold indicate, from 5′-3′, splice acceptors 4A, 4B, 5, 7A, 7B, and 7. The rev initiator ATG is underlined.

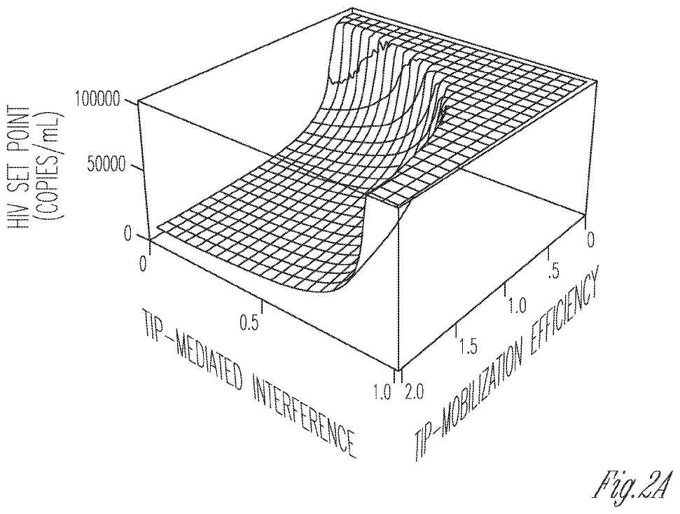

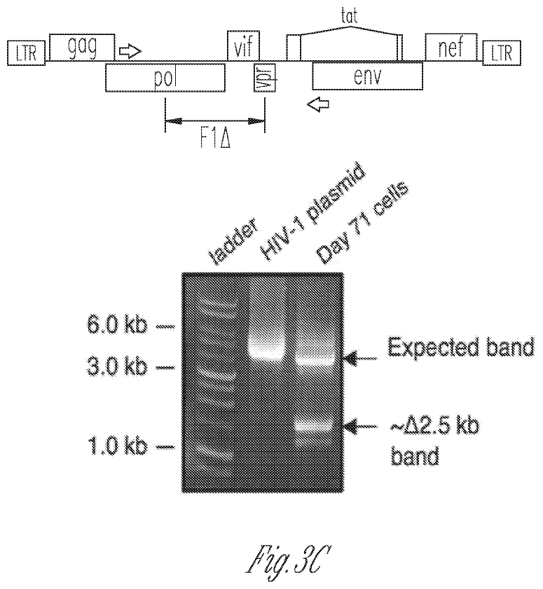

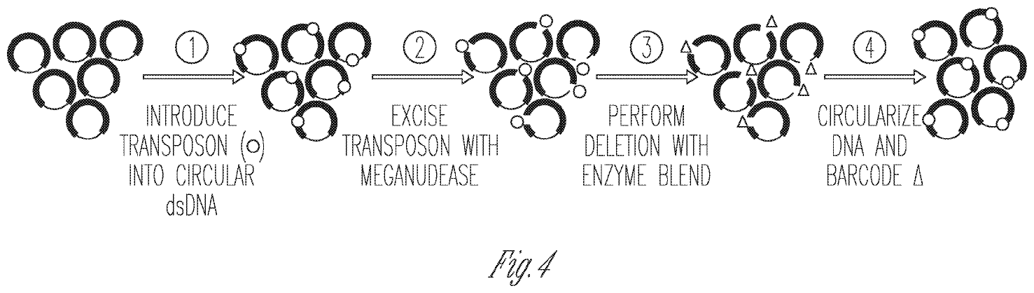

An exemplary nucleotide sequence surrounding HIV splice acceptor A7 is shown in FIG. 2A of Kammler et al. (2006) Retrovirol. 3:89. An exemplary nucleotide sequence surrounding HIV splice acceptor A5 is shown in FIG. 4A of Kammler et al. (2006) Retrovirol. 3:89. Exemplary nucleotide sequences surrounding HIV splice acceptors A1-A7 are shown in FIG. 5A of Kammler et al. (2006) Retrovirol. 3:89.

### Exemplary Interfering Constructs

Non-limiting examples of interfering constructs are depicted schematically in FIGS. 1D, 2A, and 4A. Exemplary interfering constructs are designated “F1pol” (SEQ ID NO:2); “F1pol splice” (SEQ ID NO:3); “F1vpr” (SEQ ID NO:4); and “F1vpr splice” (SEQ ID NO:5). Exemplary interfering constructs are depicted in FIGS. 7A-7E.

In certain embodiments, the interfering, conditionally replicating, human immunodeficiency virus (HIV) construct comprises one or more alterations in an HIV nucleotide sequence, wherein the one or more alterations renders each of Pol, Tat, Vpr, Nef, and Vif nonfunctional such that the construct is incapable of replication and production of virus on its own but requires replication-competent HIV to act as a helper virus.

In certain embodiments, one or more alterations in the HIV nucleotide sequence comprise a deletion or a mutation of one or more nucleotides in the nucleotide sequence encoding HIV Pol, a deletion or a mutation of one or more nucleotides in the nucleotide sequence encoding HIV Vif, a deletion or a mutation of one or more nucleotides in the nucleotide sequence encoding HIV Vpr, a deletion or a mutation of one or more nucleotides in the nucleotide sequence encoding HIV Tat, and a deletion or a mutation of one or more nucleotides in the nucleotide sequence encoding HIV Nef.

In certain embodiments, the construct comprises a deletion of the nucleotides corresponding to positions 3159 to 4780, 4904 to 5737, and 8786 to 9048 numbered relative to the reference HIV sequence of SEQ ID NO:1. In certain embodiments, the construct comprises the nucleotides corresponding to positions 1 to 634, 635 to 789, 686 to 823, 790 to 2292, 2085 to 3158, 4781 to 4903, 5738 to 5849, 5830 to 6044, 5872 to 5880, 5969 to 6044, 6045 to 6060, 6061 to 6306, 6221 to 8785, 7759 to 7992, 8369 to 8414, 8369 to 8643, and 9049 to 9709 numbered relative to the reference HIV sequence of SEQ ID NO:1.

In certain embodiments, the construct comprises the nucleotide sequence of SEQ ID NO:2, or a nucleotide sequence displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto.

In certain embodiments, the construct further comprises a deletion or a mutation of one or more nucleotides in the nucleotide sequence encoding HIV Rev, a deletion or a mutation of one or more nucleotides in the nucleotide sequence encoding HIV Vpu, and a deletion or a mutation of one or more nucleotides in the nucleotide sequence encoding HIV Env. In certain embodiments, the construct comprises a deletion of the nucleotides corresponding to positions 3159 to 4780, 4904 to 5737, 5850 to 6341, and 8786 to 9048 numbered relative to the reference HIV sequence of SEQ ID NO:1. In certain embodiments, the construct comprises the 5 nucleotides corresponding to positions 1 to 634, 635 to 789, 686 to 823, 790 to 2292, 2085 to 3158, 4781 to 4903, 5738 to 5849, 6342 to 8785, 7759 to 7992, 8369 to 8414, 8369 to 8643, and 9049 to 9709 numbered relative to the reference HIV sequence of SEQ ID NO:1. In certain embodiments, the construct comprises the nucleotide sequence of SEQ ID NO:3, or a nucleotide sequence displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto.

In certain embodiments, the construct comprises a deletion of the nucleotides corresponding to positions 3159 to 5630 and 8786 to 9048 numbered relative to the reference HIV sequence of SEQ ID NO:1. In certain embodiments, the construct comprises the nucleotides corresponding to positions 1 to 634, 635 to 789, 686 to 823, 790 to 2292, 2085 to 3158, 5631 to 5849, 4778 to 4898, 5850 to 6044, 5969 to 6044, 6045 to 6060, 6061 to 6306, 6221 to 8785, 7759 to 7992, 8369 to 8414, 8369 to 8643, 9049 to 9709 numbered relative to the reference HIV sequence of SEQ ID NO:1. In certain embodiments, the construct comprises the nucleotide sequence of SEQ ID NO:4, or a nucleotide sequence displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto.

In certain embodiments, the construct further comprises a deletion of the nucleotides corresponding to positions 5850 to 6341, numbered relative to the reference HIV sequence of SEQ ID NO:1. In certain embodiments, the construct comprises the nucleotides corresponding to positions 1 to 634, 635 to 789, 686 to 823, 790 to 2292, 2085 to 3158, 5631 to 5849, 4778 to 4898, 6342 to 8785, 7759 to 7992, 8369 to 8414, 8369 to 8643, 9049 to 9709 numbered relative to the reference HIV sequence of SEQ ID NO:1. In certain embodiments, the construct comprises the nucleotide sequence of SEQ ID NO:5, or a nucleotide sequence displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto.

The foregoing numbering is relative to the reference genomic sequence of the HIV-1 pNL4-3 strain (SEQ ID NO:1), but it is to be understood that the corresponding positions in the genomes of other HIV strains and isolates of any type, subtype (HIV-1 Groups: M, N, O, P and HIV-2), or clade (HIV-1 M: A1, A2, A3, A4, A6, B, C, D, F1, F2, G, H, J, and K; HIV-2: A, B, C, F and G), are also intended to be encompassed by the present invention.

In certain embodiments, the construct comprises a deletion in the nucleotide sequence encoding HIV Pol, a deletion of the nucleotide sequence encoding HIV Vif, a deletion of the nucleotide sequence encoding HIV Vpr, a deletion of the nucleotide sequence encoding HIV Tat, a deletion of the nucleotide sequence encoding HIV Rev, a deletion of the nucleotide sequence encoding HIV Vpu, a deletion of the nucleotide sequence encoding HIV Env, a deletion of the nucleotide sequence encoding HIV Nef, or a deletion of the nucleotide sequence encoding HIV Gag, or a combination thereof.

Interfering Particles

The present disclosure further comprises a particle comprising an interfering construct. Such a particle is referred to herein as an “interfering particle.” An interfering particle is capable of infecting and entering a host cell.

An interfering particle of the present disclosure will in some cases comprise HIV envelope proteins (e.g., gp120 and gp41). In other cases, an interfering particle of the present disclosure will comprise a non-HIV envelope protein, i.e., the interfering construct will be pseudotyped.

In some cases, an interfering particle of the present disclosure will comprise HIV envelope proteins (e.g., gp120 and gp41), such that the packaged interfering particle will infect cells via the same receptor (CCR5) used by wild-type HIV. CCR5 is predominantly expressed on T cells, macrophages, dendritic cells and microglia. Thus, in some embodiments, a subject interfering particle will infect primarily T cells, macrophages, dendritic cells and microglia.

Pseudotyped Interfering Constructs

As noted above, in some instances, a subject interfering construct will be pseudotyped. For example, in some instances, a subject interfering construct is pseudotyped with VSV-G. VSV-G pseudotyped retroviruses demonstrate a broad host range (pantropic) and are able to efficiently infect cells that are resistant to infection by ecotropic and amphotropic retroviruses. (Yee et al. (2004) Proc. Natl. Acad. Sci. USA 91:9564-9568. Any suitable serotype (e.g., Indiana, New Jersey, Chandipura, Piry) and strain (e.g., VSV Indiana, San Juan) of VSV-G can be used. Stable VSV-G pseudotyped retrovirus packaging cell lines permit generation of large scale viral preparations (e.g. from 10 to 50 liters supernatant) to yield retroviral stocks in the range of 10⁷ to 10¹¹ retroviral particles per ml.

In some embodiments, an interfering construct of the present disclosure is pseudotyped with a Sindbis virus envelope glycoprotein. See, e.g., U.S. Pat. No. 8,187,872.

Cell Lines

Any suitable cell line can be employed to prepare packaging cells for use in packaging a subject interfering construct to generate an interfering construct-containing infectious particle (an “interfering particle”). Generally, the cells are mammalian cells. In a particular embodiment, the cells used to produce the packaging cell line are human cells. Suitable human cell lines which can be used include, for example, 293 cells (Graham et al. (1977) J. Gen. Virol., 36:59-72, tsa 201 cells (Heinzel et al. (1988) J. Virol., 62:3738), and NIH3T3 cells (ATCC)). Other suitable packaging cell lines for use in the present invention include other human cell line derived (e.g., embryonic cell line derived) packaging cell lines and murine cell line derived packaging cell lines, such as Psi-2 cells (Mann et al. (1983) Cell, 33:153-159; FLY (Cossett et al. (1993) Virol., 193:385-395; BOSC 23 cells (Pear et al. (1993) PNAS 90:8392-8396; PA317 cells (Miller et al. (1986) Molec. and Cell. Biol., 6:2895-2902; Kat cell line (Finer et al. (1994) Blood, 83:43-50; GP+E cells and GP+EM12 cells (Markowitz et al. (1988) J. Virol., 62:1120-1124, and Psi Crip and Psi Cre cells (U.S. Pat. No. 5,449,614; Danos, O. and Mulligan et al. (1988) PNAS 85:6460-6464). Packaging cell lines can produce retroviral particles having a pantropic, amphotropic, or ecotropic host range. Exemplary packaging cell lines produce retroviral particles, such as lentiviral particles (e.g., HIV-1, HIV-2 and SIV) capable of infecting dividing, as well as non-dividing cells.

Compositions

The present disclosure provides compositions, including pharmaceutical compositions and biological compositions, comprising a subject interfering construct or a subject interfering particle. For simplicity, a subject interfering construct and a subject interfering particle are referred to collectively below as an “active agent.”

The present disclosure provides a composition comprising a subject interfering construct or a subject interfering particle. A subject interfering construct composition or a subject interfering particle composition can comprise, in addition to a subject interfering construct or a subject interfering particle, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a nuclease inhibitor; glycerol; and the like.

Pharmaceutical Compositions

An active agent is in some embodiments formulated with a pharmaceutically acceptable excipient(s). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc. For the purposes of the following description of formulations, “active agent” includes an active agent as described above, and optionally one or more additional therapeutic agent.

In a subject method, an active agent may be administered to the host using any convenient means capable of resulting in the desired degree of reduction of immunodeficiency virus transcription. Thus, an active agent can be incorporated into a variety of formulations for therapeutic administration. For example, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. In an exemplary embodiment, an active agent is formulated as a gel, as a solution, or in some other form suitable for intravaginal administration. In a further exemplary embodiment, an active agent is formulated as a gel, as a solution, or in some other form suitable for rectal (e.g., intrarectal) administration.

In pharmaceutical dosage forms, an active agent may be administered in the form of its pharmaceutically acceptable salts, or it may also be used alone or in appropriate association, as well as in combination, with other pharmaceutically active compounds. The following methods and excipients are merely exemplary and are in no way limiting.

In some embodiments, an active is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. An active agent can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise the active agent(s) in a composition as a solution in sterile water, normal saline or another pharmaceutically acceptable carrier.

Unit dosage forms for intravaginal or intrarectal administration such as syrups, elixirs, gels, and suspensions may be provided wherein each dosage unit, for example, teaspoonful, tablespoonful, tablet, unit gel volume, or suppository, contains a predetermined amount of the composition containing one or more active agents.

The term “unit dosage form,” as used herein, refers to physically discrete units suitable as unitary dosages for human and animal subjects, each unit containing a predetermined quantity of an active agent, calculated in an amount sufficient to produce the desired effect in association with a pharmaceutically acceptable diluent, carrier or vehicle. The specifications for a given active agent will depend in part on the particular compound employed and the effect to be achieved, and the pharmacodynamics associated with each compound in the host. Other modes of administration will also find use with the subject invention. For instance, an active agent can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), e.g. about 1% to about 2%.

An active agent can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

An active agent will in some embodiments be formulated for vaginal delivery. A subject formulation for intravaginal administration comprises an active agent formulated as an intravaginal bioadhesive tablet, intravaginal bioadhesive microparticle, intravaginal cream, intravaginal lotion, intravaginal foam, intravaginal ointment, intravaginal paste, intravaginal solution, or intravaginal gel.

An active agent will in some embodiments be formulated for rectal delivery. A subject formulation for intrarectal administration comprises an active agent formulated as an intrarectal bioadhesive tablet, intrarectal bioadhesive microparticle, intrarectal cream, intrarectal lotion, intrarectal foam, intrarectal ointment, intrarectal paste, intrarectal solution, or intrarectal gel. A subject formulation comprising an active agent includes one or more of an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Tablets comprising an active agent may be coated with a suitable film-forming agent, e.g., hydroxypropylmethyl cellulose, hydroxypropyl cellulose or ethyl cellulose, to which a suitable excipient may optionally be added, e.g., a softener such as glycerol, propylene glycol, diethylphthalate, or glycerol triacetate; a filler such as sucrose, sorbitol, xylitol, glucose, or lactose; a colorant such as titanium hydroxide; and the like.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated. The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Biological Compositions

The present disclosure provides a biological composition comprising: a) a subject interfering construct or a subject interfering particle; and b) a biological fluid. Suitable biological fluids include, e.g., blood or a blood fraction. Blood fractions include, e.g., serum and plasma. In some cases, the biological fluid has been isolated from an individual. In some cases, the biological fluid has been subjected to one or more processing steps, e.g., removal of pathogen(s) such as HCV. HIV, and the like.

Treatment and Prophylactic Methods

The present disclosure provides a method of reducing human immunodeficiency virus viral load in an individual. The method generally involves administering to the individual an effective amount of a subject interfering construct, a pharmaceutical formulation comprising a subject interfering construct, a subject interfering particle, or a pharmaceutical formulation comprising a subject interfering particle.

In some cases, a subject method involves administering to an individual in need thereof an effective amount of a subject interfering particle, or a pharmaceutical formulation comprising a subject interfering particle. In some cases, an effective amount of a subject interfering particle is an amount that, when administered to an individual in one or more doses, in monotherapy or in combination therapy, is effective to reduce immunodeficiency virus load in the individual by at least about 10%, at least about 20%, at least about 25%, at least about 30%, at least about 40%, at least about 50%, at least about 60%, at least about 70%, at least about 80%, or greater than 80%, compared to the immunodeficiency virus load in the individual in the absence of treatment with the interfering particle.

In some cases, a subject method involves administering to an individual in need thereof an effective amount of a subject interfering particle. In some embodiments, an “effective amount” of a subject interfering particle is an amount that, when administered to an individual in one or more doses, in monotherapy or in combination therapy, is effective to increase the number of CD4⁺ T cells in the individual by at least about 20%, at least about 25%, at least about 30%, at least about 40%, at least about 50%, at least about 60%, at least about 70%, at least about 80%, at least about 2-fold, at least about 2.5-fold, at least about 3-fold, at least about 5-fold, at least about 10-fold, or greater than 10-fold, compared to the number of CD4⁺ T cells in the individual in the absence of treatment with the interfering particle.

Any of a variety of methods can be used to determine whether a treatment method is effective. For example, methods of determining whether the methods of the invention are effective in reducing immunodeficiency virus (e.g., HIV) viral load, and/or treating an immunodeficiency virus (e.g., HIV) infection, are any known test for indicia of immunodeficiency virus (e.g., HIV) infection, including, but not limited to, measuring viral load, e.g., by measuring the amount of immunodeficiency virus (e.g., HIV) in a biological sample, e.g., using a polymerase chain reaction (PCR) with primers specific for an immunodeficiency virus (e.g., HIV) polynucleotide sequence: detecting and/or measuring a polypeptide encoded by an immunodeficiency virus (e.g., HIV), e.g., p24, gp120, reverse transcriptase, using, e.g., an immunological assay such as an enzyme-linked immunosorbent assay (ELISA) with an antibody specific for the polypeptide; and measuring the CD4⁺ T cell count in the individual.

Formulations, Dosages, and Routes of Administration

Prior to introduction into a host, an interfering construct or an interfering particle can be formulated into various compositions for use in therapeutic and prophylactic treatment methods. In particular, the interfering construct or interfering particle can be made into a pharmaceutical composition by combination with appropriate pharmaceutically acceptable carriers or diluents, and can be formulated to be appropriate for either human or veterinary applications. For simplicity, a subject interfering construct and a subject interfering particle are collectively referred to below as “active agent” or “active ingredient.”

Thus, a composition for use in a subject treatment method can comprise a subject interfering construct or subject interfering particle, e.g., in combination with a pharmaceutically acceptable carrier. Pharmaceutically acceptable carriers are well known to those skilled in the art, as are suitable methods of administration. The choice of carrier will be determined, in part, by the particular vector, as well as by the particular method used to administer the composition. One skilled in the art will also appreciate that various routes of administering a composition are available, and, although more than one route can be used for administration, a particular route can provide a more immediate and more effective reaction than another route. Accordingly, there are a wide variety of suitable formulations of a subject interfering construct composition or a subject interfering particle composition.

A composition a subject interfering construct or subject interfering particle, alone or in combination with other antiviral compounds, can be made into a formulation suitable for parenteral administration. Such a formulation can include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The formulations can be provided in unit dose or multidose sealed containers, such as ampules and vials, and can be stored in a freeze-dried (lyophilized) condition requiring only the addition of the sterile liquid carrier, for example, water, for injections, immediately prior to use. Injectable solutions and suspensions can be prepared from sterile powders, granules, and tablets, as described herein.

A formulation suitable for oral administration can be a liquid solution, such as an effective amount of a subject interfering construct or a subject interfering particle dissolved in diluents, such as water, saline, or fruit juice; capsules, sachets or tablets, each containing a predetermined amount of the active agent (a subject interfering construct or subject interfering particle), as solid or granules; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers.

An aerosol formulation suitable for administration via inhalation also can be made. The aerosol formulation can be placed into a pressurized acceptable propellant, such as dichlorodifluoromethane, propane, nitrogen, and the like.

Similarly, a formulation suitable for oral administration can include lozenge forms, that can comprise the active ingredient in a flavor, usually sucrose and acacia or tragacanth; pastilles comprising the active ingredient (a subject interfering construct or subject interfering particle) in an inert base, such as gelatin and glycerin, or sucrose and acacia; and mouthwashes comprising the active agent in a suitable liquid carrier; as well as creams, emulsions, gels, and the like containing, in addition to the active agent, such carriers as are known in the art.

A formulation suitable for topical application can be in the form of creams, ointments, or lotions.

A formulation for rectal administration can be presented as a suppository with a suitable base comprising, for example, cocoa butter or a salicylate. A formulation suitable for vaginal administration can be presented as a pessary, tampon, cream, gel, paste, foam, or spray formula containing, in addition to the active ingredient, such carriers as are known in the art to be appropriate. Similarly, the active ingredient can be combined with a lubricant as a coating on a condom.

The dose administered to an animal, particularly a human, in the context of the present invention should be sufficient to effect a therapeutic response in the infected individual over a reasonable time frame. The dose will be determined by the potency of the particular interfering construct or interfering particle employed for treatment, the severity of the disease state, as well as the body weight and age of the infected individual. The size of the dose also will be determined by the existence of any adverse side effects that can accompany the use of the particular interfering construct or interfering particle employed. It is always desirable, whenever possible, to keep adverse side effects to a minimum.

The dosage can be in unit dosage form, such as a tablet, a capsule, a unit volume of a liquid formulation, etc. The term “unit dosage form” as used herein refers to physically discrete units suitable as unitary dosages for human and animal subjects, each unit containing a predetermined quantity of an interfering construct or an interfering particle, alone or in combination with other antiviral agents, calculated in an amount sufficient to produce the desired effect in association with a pharmaceutically acceptable diluent, carrier, or vehicle. The specifications for the unit dosage forms of the present disclosure depend on the particular construct or particle employed and the effect to be achieved, as well as the pharmacodynamics associated with each construct or particle in the host. The dose administered can be an “antiviral effective amount” or an amount necessary to achieve an “effective level” in the individual patient.

Generally, an amount of a subject interfering construct or a subject interfering particle sufficient to achieve a tissue concentration of the administered construct or particle of from about 50 mg/kg to about 300 mg/kg of body weight per day can be administered, e.g., an amount of from about 100 mg/kg to about 200 mg/kg of body weight per day. In certain applications, e.g., topical, ocular or vaginal applications, multiple daily doses can be administered. Moreover, the number of doses will vary depending on the means of delivery and the particular interfering construct or interfering particle administered.

Combination Therapy

In some embodiments, a subject interfering construct or interfering particle (or composition comprising same) is administered in combination therapy with one or more additional therapeutic agents. Suitable additional therapeutic agents include agents that inhibit one or more functions of an immunodeficiency virus; agents that treat or ameliorate a symptom of an immunodeficiency virus infection; agents that treat an infection that may occur secondary to an immunodeficiency virus infection; and the like.

Therapeutic agents include, e.g., beta-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™), trisodium phosphonoformate (Foscarnet™), famcyclovir, pencyclovir, valacyclovir, nucleic acid/replication inhibitors, interferon, zidovudine (AZT, Retrovir™), didanosine (dideoxyinosine, ddI, Videx™), stavudine (d4T, Zerit™), zalcitabine (dideoxycytosine, ddC, Hivid™), nevirapine (Viramune™), lamivudine (Epivir™, 3TC), protease inhibitors, saquinavir (Invirase™, Fortovase™), ritonavir (Norvir™), nelfinavir (Viracept™), efavirenz (Sustiva™), abacavir (Ziagen™), amprenavir (Agenerase™) indinavir (Crixivan™), ganciclovir, AzDU, delavirdine (Rescriptor™), kaletra, trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparaginase and combinations thereof. Anti-HIV agents are those in the preceding list that specifically target a function of one or more HIV proteins.

In some embodiments, a subject active agent is administered in combination therapy with two or more anti-HIV agents. For example, a subject active agent can be administered in combination therapy with one, two, or three nucleoside reverse transcriptase inhibitors (e.g., Combivir, Epivir, Hivid, Retrovir, Videx, Zerit, Ziagen, etc.). A subject active agent can be administered in combination therapy with one or two non-nucleoside reverse transcriptase inhibitors (e.g., Rescriptor, Sustiva, Viramune, etc.). A subject active agent can be administered in combination therapy with one or two protease inhibitors (e.g., Agenerase, Crixivan, Fortovase, Invirase, Kaletra, Norvir, Viracept, etc.). A subject active agent can be administered in combination therapy with a protease inhibitor and a nucleoside reverse transcriptase inhibitor. A subject active agent can be administered in combination therapy with a protease inhibitor, a nucleoside reverse transcriptase inhibitor, and a non-nucleoside reverse transcriptase inhibitor. A subject active agent can be administered in combination therapy with a protease inhibitor and a non-nucleoside reverse transcriptase inhibitor. Other combinations of a subject active agent with one or more of a protease inhibitor, a nucleoside reverse transcriptase inhibitor, and a non-nucleoside reverse transcriptase inhibitor are contemplated.

In some embodiments, a subject treatment method involves administering: a) a subject active agent; and b) an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity.

In some embodiments, a subject treatment method involves administering: a) a subject active agent; and b) an HIV inhibitor, where suitable HIV inhibitors include, but are not limited to, one or more nucleoside/nucleotide reverse transcriptase inhibitors (NRTIs), non-nucleoside reverse transcriptase inhibitors (NNRTIs), protease inhibitors (PIs), fusion inhibitors, integrase inhibitors, chemokine receptor (e.g., CXCR4, CCR5) inhibitors, and hydroxyurea. Nucleoside reverse transcriptase inhibitors include, but are not limited to, abacavir (ABC; ZIAGEN™), didanosine (dideoxyinosine (ddI); VIDEX™), lamivudine (3TC; EPIVIR™), stavudine (d4T: ZERIT™, ZERIT XR™), zalcitabine (dideoxycytidine (ddC): HMD™), zidovudine (ZDV, formerly known as azidothymidine (AZT); RETROVIR™), abacavir, zidovudine, and lamivudine (TRIZIVIR™), zidovudine and lamivudine (COMBIVIR™), and emtricitabine (EMTRIVA™). Nucleotide reverse transcriptase inhibitors include tenofovir disoproxil fumarate (VIREAD™). Non-nucleoside reverse transcriptase inhibitors for HIV include, but are not limited to, nevirapine (VIRAMUNE™), delavirdine mesylate (RESCRIPTOR™), and efavirenz (SUSTIVA™).

Protease inhibitors (PIs) for treating HIV infection include amprenavir (AGENERASE™), saquinavir mesylate (FORTOVASE™, INVIRASE™), ritonavir (NORVIR™), indinavir sulfate (CRIXIVAN™), nelfmavir mesylate (VIRACEPT™), lopinavir and ritonavir (KALETRA™), atazanavir (REYATAZ™), and fosamprenavir (LEXIVA™). Fusion inhibitors prevent fusion between the virus and the cell from occurring, and therefore, prevent HIV infection and multiplication. Fusion inhibitors include, but are not limited to, enfuvirtide (FUZEON™), Lalezari et al., New England J. Med., 348:2175-2185 (2003); and maraviroc (SELZENTRY™, Pfizer).

An integrase inhibitor blocks the action of integrase, preventing HIV-1 genetic material from integrating into the host DNA, and thereby stopping viral replication. Integrase inhibitors include, but are not limited to, raltegravir (ISENTRESS™, Merck); and elvitegravir (GS 9137, Gilead Sciences).

Maturation inhibitors include, e.g., bevirimat (3β-(3-carboxy-3-methyl-butanoyloxy) lup-20(29)-en-28-oic acid); and Vivecon (MPC9055).

In some embodiments, a subject treatment method involves administering: a) a subject active agent; and b) one or more of: (1) an HIV protease inhibitor selected from amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, RO0334649, KNI-272, DPC-681, DPC-684, GW640385X, DG17, PPL-100, DG35, and AG 1859; (2) an HIV non-nucleoside inhibitor of reverse transcriptase selected from capravirine, emivirine, delaviridine, efavirenz, nevirapine, (+) calanolide A, etravirine, GW5634, DPC-083, DPC-961, DPC-963, MIV-150, and TMC-120, TMC-278 (rilpivirene), efavirenz, BILR 355 BS, VRX 840773, UK-453061, and RDEA806; (3) an HIV nucleoside inhibitor of reverse transcriptase selected from zidovudine, emtricitabine, didanosine, stavudine, zalcitabine, lamivudine, abacavir, amdoxovir, elvucitabine, alovudine, MIV-210, racivir, D-d4FC, emtricitabine, phosphazide, fozivudine tidoxil, apricitibine (AVX754), amdoxovir, KP-1461, and fosalvudine tidoxil (formerly HDP 99.0003); (4) an HIV nucleotide inhibitor of reverse transcriptase selected from tenofovir and adefovir; (5) an HIV integrase inhibitor selected from curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, MK-0518 (raltegravir), BMS-538158, GSK364735C, BMS-707035, MK-2048, and BA 011; (6) a gp41 inhibitor selected from enfuvirtide, sifuvirtide, FB006M, and TRI-1144; (7) a CXCR4 inhibitor, such as AMD-070; (8) an entry inhibitor, such as SP01A; (9) a gp120 inhibitor, such as BMS-488043 and/or BlockAide/CR; (10) a G6PD and NADH-oxidase inhibitor, such as immunitin; (11) a CCR5 inhibitors selected from the group consisting of aplaviroc, vicriviroc, maraviroc, PRO-140, 1NCB15050, PF-232798 (Pfizer), and CCR5 mAb004; (12) another drug for treating HIV selected from BAS-100, SPI-452, REP 9, SP-01A, TNX-355, DES6, ODN-93, ODN-112, VGV-1, PA-457 (bevirimat), Ampligen, HRG214, Cytolin, VGX-410, KD-247, AMZ 0026, CYT 99007A-221 HIV, DEBIO-025, BAY 50-4798, MDXO10 (ipilimumab), PBS119, ALG 889, and PA-1050040 (PA-040); (13) any combinations or mixtures of the above.

As further examples, in some embodiments, a subject treatment method involves administering: a) a subject active agent; and b) one or more of: i) amprenavir (Agenerase; (3S)-oxolan-3-yl N-[(2S,3R)-3-hydroxy-4-[N-(2-methylpropyl)(4-aminobenzene)sulfonamido]-1-phenylbutan-2-yl]carbamate) in an amount of 600 mg or 1200 mg twice daily; ii) tipranavir (Aptivus; N-{3-[(1R)-1-[(2R)-6-hydroxy-4-oxo-2-(2-phenylethyl)-2-propyl-3,4-dihydro-2H-pyran-5-yl]propyl]phenyl}-5-(trifluoromethyl)pyridine-2-sulfonamide) in an amount of 500 mg twice daily: iii) idinavir (Crixivan; (2S)-1-[(2S,4R)-4-benzyl-2-hydroxy-4-{[(1S,2R)-2-hydroxy-2,3-dihydro-1H-inden-1-yl]carbamoyl}butyl]-N-tert-butyl-4-(pyridin-3-ylmethyl)piperazine-2-carboxamide) in an amount of 800 mg three times daily; iv) saquinavir (Invirase; 2S)-N-[(2S,3R)-4-[(3S)-3-(tert-butylcarbamoyl)-decahydroisoquinolin-2-yl]-3-hydroxy-1-phenylbutan-2-yl]-2-(quinolin-2-ylformamido)butanediamide) in an amount of 1,000 mg twice daily; v) lopinavir and ritonavir (Kaleta; where lopinavir is 2S)-N-[(2S,4S,5S)-5-[2-(2,6-dimethylphenoxy)acetamido]-4-hydroxy-1,6-diphenylhexan-2-yl]-3-methyl-2-(2-oxo-1,3-diazinan-1-yl)butanamide; and ritonavir is 1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl})carbamoyl]amino}butanamido]-1,6-diphenylhexan-2-yl]carbamate) in an amount of 133 mg twice daily: vi) fosamprenavir (Lexiva: {[(2R,3S)-1-[N-(2-methylpropyl)(4-aminobenzene)sulfonamido]-3-({[(3S)-oxolan-3-yloxy]carbonyl}amino)-4-phenylbutan-2-yl]oxy}phosphonic acid) in an amount of 700 mg or 1400 mg twice daily): vii) ritonavir (Norvir) in an amount of 600 mg twice daily; viii) nelfinavir (Viracept: (3S,4aS,8aS)-N-tert-butyl-2-[(2R,3R)-2-hydroxy-3-[(3-hydroxy-2-methylphenyl)formamido]-4-(phenylsulfanyl)butyl]-decahydroisoquinoline-3-carboxamide) in an amount of 750 mg three times daily or in an amount of 1250 mg twice daily; ix) Fuzeon (Acetyl-YTSLIHSLIEESQNQ QEKNEQELLELDKWASLWNWF-amide; SEQ ID NO:20) in an amount of 90 mg twice daily; x) Combivir in an amount of 150 mg lamivudine (3TC; 2′,3′-dideoxy-3′-thiacytidine) and 300 mg zidovudine (AZT; azidothymidine) twice daily; xi) emtricitabine (Emtriva; 4-amino-5-fluoro-1-[(2R,5S)-2-(hydroxymethyl)-1,3-oxathiolan-5-yl]-1,2-dihydropyrimidin-2-one) in an amount of 200 mg once daily; xii) Epzicom in an amount of 600 mg abacavir (ABV; {(1S,4R)-4-[2-amino-6-(cyclopropylamino)-9H-purin-9-yl]cyclopent-2-en-1-yl}methanol) and 300 mg 3TC once daily; xiii) zidovudine (Retrovir; AZT or azidothymidine) in an amount of 200 mg three times daily: xiv) Trizivir in an amount of 150 mg 3TC and 300 mg ABV and 300 mg AZT twice daily: xv) Truvada in an amount of 200 mg emtricitabine and 300 mg tenofovir (({[(2R)-1-(6-amino-9H-purin-9-yl)propan-2-yl]oxy}methyl)phosphonic acid) once daily: xvi) didanosine (Videx; 2′,3′-dideoxyinosine) in an amount of 400 mg once daily; xvii) tenofovir (Viread) in an amount of 300 mg once daily; xviii) abacavir (Ziagen) in an amount of 300 mg twice daily; xix) atazanavir (Reyataz; methyl N-[(1S)-1-{[(2S,3S)-3-hydroxy-4-[(2S)-2-[(methoxycarbonyl)amino]-3,3-dimethyl-N′-{[4-(pyridin-2-yl)phenyl]methyl}butanehydrazido]-1-phenylbutan-2-yl]carbamoyl}-2,2-dimethylpropyl]carbamate) in an amount of 300 mg once daily or 400 mg once daily; xx) lamivudine (Epivir) in an amount of 150 mg twice daily; xxi) stavudine (Zerit; 2′-3′-didehydro-2′-3′-dideoxythymidine) in an amount of 40 mg twice daily; xxii) delavirdine (Rescriptor; N-[2-({4-[3-(propan-2-ylamino)pyridin-2-yl]piperazin-1-yl}carbonyl)-1H-indol-5-yl]methanesulfonamide) in an amount of 400 mg three times daily; xxiii) efavirenz (Sustiva; (4S)-6-chloro-4-(2-cyclopropylethynyl)-4-(trifluoromethyl)-2,4-dihydro-1H-3,1-benzoxazin-2-one) in an amount of 600 mg once daily); xxiv) nevirapine (Viramune; 11-cyclopropyl-4-methyl-5,11-dihydro-6H-dipyrido[3,2-b:2′,3′-e][1,4]diazepin-6-one) in an amount of 200 mg twice daily); xxv) bevirimat; and xxvi) Vivecon.

Kits, Containers, Devices, Delivery Systems

Kits with unit doses of the active agent, e.g. in oral, vaginal, rectal, transdermal, or injectable doses (e.g., for intramuscular, intravenous, or subcutaneous injection), are provided. In such kits, in addition to the containers containing the unit doses will be an informational package insert describing the use and attendant benefits of the drugs in treating an immunodeficiency virus (e.g., an HIV) infection. Suitable active agents (a subject interfering construct or a subject interfering particle) and unit doses are those described herein above. In many embodiments, a subject kit will further include instructions for practicing the subject methods or means for obtaining the same (e.g., a website URL directing the user to a webpage which provides the instructions), where these instructions are typically printed on a substrate, which substrate may be one or more of: a package insert, the packaging, formulation containers, and the like.

In some embodiments, a subject kit includes one or more components or features that increase patient compliance, e.g., a component or system to aid the patient in remembering to take the active agent at the appropriate time or interval. Such components include, but are not limited to, a calendaring system to aid the patient in remembering to take the active agent at the appropriate time or interval.

The present invention provides a delivery system comprising an active agent. In some embodiments, the delivery system is a delivery system that provides for injection of a formulation comprising an active agent subcutaneously, intravenously, or intramuscularly. In other embodiments, the delivery system is a vaginal or rectal delivery system. In some embodiments, an active agent is packaged for oral administration. The present invention provides a packaging unit comprising daily dosage units of an active agent. For example, the packaging unit is in some embodiments a conventional blister pack or any other form that includes tablets, pills, and the like. The blister pack will contain the appropriate number of unit dosage forms, in a sealed blister pack with a cardboard, paperboard, foil, or plastic backing, and enclosed in a suitable cover. Each blister container may be numbered or otherwise labeled, e.g., starting with day 1.

In some embodiments, a subject delivery system comprises an injection device. Exemplary, non-limiting drug delivery devices include injections devices, such as pen injectors, and needle/syringe devices. In some embodiments, the invention provides an injection delivery device that is pre-loaded with a formulation comprising an effective amount of a subject active agent. For example, a subject delivery device comprises an injection device pre-loaded with a single dose of a subject active agent. A subject injection device can be re-usable or disposable. Pen injectors are well known in the art. Exemplary devices which can be adapted for use in the present methods are any of a variety of pen injectors from Becton Dickinson, e.g., BD™ Pen, BD™ Pen II, BD™ Auto-Injector; a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable.

The present invention provides a delivery system for vaginal or rectal delivery of an active agent to the vagina or rectum of an individual. The delivery system comprises a device for insertion into the vagina or rectum. In some embodiments, the delivery system comprises an applicator for delivery of a formulation into the vagina or rectum; and a container that contains a formulation comprising an active agent. In these embodiments, the container (e.g., a tube) is adapted for delivering a formulation into the applicator. In other embodiments, the delivery system comprises a device that is inserted into the vagina or rectum, which device includes an active agent. For example, the device is coated with, impregnated with, or otherwise contains a formulation comprising the active agent.

In some embodiments, the vaginal or rectal delivery system is a tampon or tampon-like device that comprises a subject formulation. Drug delivery tampons are known in the art, and any such tampon can be used in conjunction with a subject drug delivery system. Drug delivery tampons are described in, e.g., U.S. Pat. No. 6,086,909. If a tampon or tampon-like device is used, there are numerous methods by which an active agent can be incorporated into the device. For example, the active agent can be incorporated into a gel-like bioadhesive reservoir in the tip of the device. Alternatively, the active agent can be in the form of a powdered material positioned at the tip of the tampon. The active agent can also be absorbed into fibers at the tip of the tampon, for example, by dissolving the active agent in a pharmaceutically acceptable carrier and absorbing the drug solution into the tampon fibers. The active agent can also be dissolved in a coating material which is applied to the tip of the tampon. Alternatively, the active agent can be incorporated into an insertable suppository which is placed in association with the tip of the tampon.

In other embodiments, the drug delivery device is a vaginal or rectal ring. Vaginal or rectal rings usually consist of an inert elastomer ring coated by another layer of elastomer containing an active agent to be delivered. The rings can be easily inserted, left in place for the desired period of time (e.g., up to 7 days), then removed by the user. The ring can optionally include a third, outer, rate-controlling elastomer layer which contains no active agent. Optionally, the third ring can contain a second active agent for a dual release ring. The active agent can be incorporated into polyethylene glycol throughout the silicone elastomer ring to act as a reservoir for drug to be delivered.

In other embodiments, a subject vaginal or rectal delivery system is a vaginal or rectal sponge. The active agent is incorporated into a silicone matrix which is coated onto a cylindrical drug-free polyurethane sponge, as described in the literature.

Pessaries, tablets, and suppositories are other examples of drug delivery systems which can be used in the context of the present disclosure. These systems have been described extensively in the literature.

Bioadhesive microparticles constitute still another drug delivery system suitable for use in the context of the present disclosure. This system is a multi-phase liquid or semi-solid preparation which does not seep from the vagina or rectum as do many suppository formulations. The substances cling to the wall of the vagina or rectum and release the drug over a period of time. Many of these systems were designed for nasal use but can be used in the vagina or rectum as well (e.g. U.S. Pat. No. 4,756,907). The system may comprise microspheres with an active agent; and a surfactant for enhancing uptake of the drug. The microparticles have a diameter of 10-100 μm and can be prepared from starch, gelatin, albumin, collagen, or dextran.

Another system is a container comprising a subject formulation (e.g., a tube) that is adapted for use with an applicator. The active agent is incorporated into creams, lotions, foams, paste, ointments, and gels which can be applied to the vagina or rectum using an applicator. Processes for preparing pharmaceuticals in cream, lotion, foam, paste, ointment and gel formats can be found throughout the literature. An example of a suitable system is a standard fragrance free lotion formulation containing glycerol, ceramides, mineral oil, petrolatum, parabens, fragrance and water such as the product sold under the trademark JERGENS™ (Andrew Jergens Co., Cincinnati, Ohio). Suitable nontoxic pharmaceutically acceptable systems for use in the compositions of the present invention will be apparent to those skilled in the art of pharmaceutical formulations and examples are described in Remington's Pharmaceutical Sciences, 19th Edition, A. R. Gennaro, ed., 1995. The choice of suitable carriers will depend on the exact nature of the particular vaginal or rectal dosage form desired, e.g., whether the active ingredient(s) is/are to be formulated into a cream, lotion, foam, ointment, paste, solution, or gel, as well as on the identity of the active ingredient(s). Other suitable delivery devices are those described in U.S. Pat. No. 6,476,079.

Subjects Suitable for Treatment

The methods of the present disclosure are suitable for treating individuals who have an immunodeficiency virus infection, e.g., who have been diagnosed as having an immunodeficiency virus infection. The methods of the present disclosure are also suitable for use in individuals who have not been diagnosed as having an HIV infection (e.g., individuals who have been tested for HIV and who have tested negative for HIV; and individuals who have not been tested), and who are considered at greater risk than the general population of contracting an HIV infection (e.g., “at risk” individuals).

The methods of the present disclosure are suitable for treating individuals who have an HIV infection (e.g., who have been diagnosed as having an HIV infection), and individuals who are considered at greater risk than the general population of contracting an HIV infection. Such individuals include, but are not limited to, individuals with healthy, intact immune systems, but who are at risk for becoming HIV infected (“at-risk” individuals). At-risk individuals include, but are not limited to, individuals who have a greater likelihood than the general population of becoming HIV infected. Individuals at risk for becoming HIV infected include, but are not limited to, individuals at risk for HIV infection due to sexual activity with HIV-infected individuals. Individuals suitable for treatment include individuals infected with, or at risk of becoming infected with, HIV-1 and/or HIV-2 and/or HIV-3, or any variant thereof.

Methods of Generating a Variant

The present disclosure provides a method of generating a variant interfering, conditionally replicating, HIV construct. The method generally involves: a) introducing an interfering construct as described above into a first individual; b) obtaining a biological sample from a second individual to whom the interfering construct has been transmitted from the first individual (either directly or via one or more intervening individuals), wherein the construct present in the second individual is a variant of the interfering construct introduced into the first individual; and c) cloning the variant construct from the second individual.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-78 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

Aspect 1. An interfering, conditionally replicating, human immunodeficiency virus (HIV) construct, the construct comprising:
a) cis-acting elements comprising a long terminal repeat, Gag-leader sequence, Ψ packaging signal, central polypurine tract (cPPT), rev response element (RRE) sequence, polypurine tract (PPT), major splice donor (MSD). A3, and A7; and
b) one or more alterations in an HIV nucleotide sequence, wherein the one or more alterations renders each of Pol, Tat, Vpr, Nef, and Vif nonfunctional such that the construct is incapable of replication and production of virus on its own but requires replication-competent HIV to act as a helper virus.

Aspect 2. The construct of aspect 1, wherein the cis-acting elements further comprise D4, A4, and A5.

Aspect 3. The construct of aspect 1 or b), wherein the long terminal repeat is a 3′ long terminal repeat or a 5′ long terminal repeat.

Aspect 4. The construct of any one of aspects 1-3, further comprising one or more alterations that renders each of Rev, Vpu, and Env nonfunctional.

Aspect 5. The construct of any one of aspects 1-4, wherein genomic RNA (gRNA) encoded by the construct is produced at a higher rate than wild-type HIV gRNA when present in a host cell infected with a wild-type HIV, such that the ratio of the gRNA encoded by the construct to the wild-type HIV gRNA is higher than about 1 in the cell.

Aspect 6. The construct of aspect 5, wherein the construct has a higher transmission frequency than the wild-type HIV.

Aspect 7. The construct of any one of aspects 1-5, wherein the construct has a basic reproductive ratio (R0)>1.

Aspect 8. The construct of any one of aspects 1-7, wherein the construct does not include any heterologous nucleotide sequences that encode a gene product.

Aspect 9. The construct of any one of aspects 1-8, wherein the construct is packaged with a higher efficiency than wild-type HIV when present in a host cell infected with a wild-type HIV.

Aspect 10. The construct of any one of aspects 1-9, wherein the cis-acting elements include at least one cis element embedded within an HIV protein-coding sequence.

Aspect 11. The construct of any one of aspects 1-10, further comprising one or more alterations comprising a deletion or mutation in an HIV splice donor site.

Aspect 12. The construct of aspect 11, wherein D2 and D3 splice donor sites are deleted.

Aspect 13. The construct of any one of aspects 1-12, further comprising one or more alterations comprising a deletion or mutation in an HIV splice acceptor site.

Aspect 14. The construct of aspect 13, wherein A1 and A2 splice acceptor sites are deleted.

Aspect 15. The construct of any one of aspects 1-14, wherein the one or more alterations in the HIV nucleotide sequence comprises a deletion or a mutation of one or more nucleotides in the nucleotide sequence encoding HIV Pol, a deletion or a mutation of one or more nucleotides in the nucleotide sequence encoding HIV Vif, a deletion or a mutation of one or more nucleotides in the nucleotide sequence encoding HIV Vpr, a deletion or a mutation of one or more nucleotides in the nucleotide sequence encoding HIV Tat, and a deletion or a mutation of one or more nucleotides in the nucleotide sequence encoding HIV Nef.

Aspect 16. The construct of aspect 15, wherein the construct comprises a deletion of the nucleotides corresponding to positions 3159 to 4780, 4904 to 5737, and 8786 to 9048 numbered relative to the reference HIV sequence of SEQ ID NO:1.

Aspect 17. The construct of aspect 16, wherein the construct comprises the nucleotides corresponding to positions 1 to 634, 635 to 789, 686 to 823, 790 to 2292, 2085 to 3158, 4781 to 4903, 5738 to 5849, 5830 to 6044, 5872 to 5880, 5969 to 6044, 6045 to 6060, 6061 to 6306, 6221 to 8785, 7759 to 7992, 8369 to 8414, 8369 to 8643, and 9049 to 9709 numbered relative to the reference HIV sequence of SEQ ID NO:1.

Aspect 18. The construct of aspect 16 or 17, wherein the construct comprises or consists of the nucleotide sequence of SEQ ID NO:2, or a nucleotide sequence having at least 90% identity to the nucleotide sequence of SEQ ID NO:2.

Aspect 19. The construct of any one of aspects 15-18, further comprising a deletion or a mutation of one or more nucleotides in the nucleotide sequence encoding HIV Rev, a deletion or a mutation of one or more nucleotides in the nucleotide sequence encoding HIV Vpu, and a deletion or a mutation of one or more nucleotides in the nucleotide sequence encoding HIV Env.

Aspect 20. The construct of aspect 19, wherein the construct comprises a deletion of the nucleotides corresponding to positions 3159 to 4780, 4904 to 5737, 5850 to 6341, and 8786 to 9048 numbered relative to the reference HIV sequence of SEQ ID NO:1.

Aspect 21. The construct of aspect 20, wherein the construct comprises the nucleotides corresponding to positions 1 to 634, 635 to 789, 686 to 823, 790 to 2292, 2085 to 3158, 4781 to 4903, 5738 to 5849, 6342 to 8785, 7759 to 7992, 8369 to 8414, 8369 to 8643, and 9049 to 9709 numbered relative to the reference HIV sequence of SEQ ID NO:1.

Aspect 22. The construct of aspect 20 or 21, wherein the construct comprises or consists of the nucleotide sequence of SEQ ID NO:3, or a nucleotide sequence having at least 90% identity to the nucleotide sequence of SEQ ID NO:3.

Aspect 23. The construct of any of aspects 1-15, wherein the construct comprises a deletion of the nucleotides corresponding to positions 3159 to 5630 and 8786 to 9048 numbered relative to the reference HIV sequence of SEQ ID NO:1.

Aspect 24. The construct of aspect 23, wherein the construct comprises the nucleotides corresponding to positions 1 to 634, 635 to 789, 686 to 823, 790 to 2292, 2085 to 3158, 5631 to 5849, 4778 to 4898, 5850 to 6044, 5969 to 6044, 6045 to 6060, 6061 to 6306, 6221 to 8785, 7759 to 7992, 8369 to 8414, 8369 to 8643, 9049 to 9709 numbered relative to the reference HIV sequence of SEQ ID NO:1.

Aspect 25. The construct of aspect 23 or 24, wherein the construct comprises or consists of the nucleotide sequence of SEQ ID NO:4, or a nucleotide sequence having at least 95% identity to the nucleotide sequence of SEQ ID NO:4.

Aspect 26. The construct of any of aspects 23-25, further comprising a deletion of the nucleotides corresponding to positions 5850 to 6341, numbered relative to the reference HIV sequence of SEQ ID NO:1.

Aspect 27. The construct of aspect 26, wherein the construct comprises the nucleotides corresponding to positions 1 to 634, 635 to 789, 686 to 823, 790 to 2292, 2085 to 3158, 5631 to 5849, 4778 to 4898, 6342 to 8785, 7759 to 7992, 8369 to 8414, 8369 to 8643, 9049 to 9709 numbered relative to the reference HIV sequence of SEQ ID NO:1.

Aspect 28. The construct of aspect 26 or 27, wherein the construct comprises or consists of the nucleotide sequence of SEQ ID NO:5, or a nucleotide sequence having at least 90% identity to the nucleotide sequence of SEQ ID NO:5.

Aspect 29. The construct of any one of aspects 1-28, wherein the construct comprises a deletion in the nucleotide sequence encoding HIV Pol.

Aspect 30. The construct of any one of aspects 1-29, wherein the construct comprises a deletion of the nucleotide sequence encoding HIV Vif.

Aspect 31. The construct of any one of aspects 1-30, wherein the construct comprises a deletion of the nucleotide sequence encoding HIV Vpr.

Aspect 32. The construct of any one of aspects 1-31, wherein the construct comprises a deletion of the nucleotide sequence encoding HIV Tat.

Aspect 33. The construct of any one of aspects 1-32, wherein the construct comprises a deletion of the nucleotide sequence encoding HIV Rev.

Aspect 34. The construct of any one of aspects 1-33, wherein the construct comprises a deletion of the nucleotide sequence encoding HIV Vpu.

Aspect 35. The construct of any one of aspects 1-34, wherein the construct comprises a deletion of the nucleotide sequence encoding HIV Env.

Aspect 36. The construct of any one of aspects 1-35, wherein the construct comprises a deletion of the nucleotide sequence encoding HIV Nef.

Aspect 37. The construct of any one of aspects 1-36, wherein the construct comprises a deletion of the nucleotide sequence encoding HIV Gag.

Aspect 38. A pharmaceutical composition comprising the construct of any one of aspects 1-37 and a pharmaceutically acceptable excipient.

Aspect 39. A method of treating an infection by a human immunodeficiency virus, the method comprising administering a therapeutically effective amount of the pharmaceutical composition of aspect 38 to an individual.

Aspect 40. A method of reducing human immunodeficiency virus viral load in an individual, the method comprising administering to the individual an effective amount of the pharmaceutical composition of aspect 38.

Aspect 41. A particle comprising the construct of any one of aspects 1-37 and a viral envelope protein.

Aspect 42. The particle of aspect 41, wherein the envelope protein comprises gp120.

Aspect 43. The particle of aspect 41, wherein the envelope protein is a non-HIV protein.

Aspect 44. A pharmaceutical composition comprising the particle of any one of aspects 41-44 and a pharmaceutically acceptable excipient.

Aspect 45. A kit for treating an infection by a human immunodeficiency virus comprising a container comprising the pharmaceutical composition of aspect 38 or 44.

Aspect 46. The kit of aspect 45, wherein the container is a syringe.

Aspect 47. A method of treating an infection by a human immunodeficiency virus, the method comprising administering a therapeutically effective amount of the pharmaceutical composition of aspect 44 to an individual.

Aspect 48. A method of reducing human immunodeficiency virus viral load in an individual, the method comprising administering to the individual an effective amount of the pharmaceutical composition of aspect 44.

Aspect 49. The method of any one of aspects 39, 40, 47, and 48, further comprising administering to the individual an effective amount of an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, viral Pol activity, and viral Vif activity.

Aspect 50. The method of aspect 49, wherein the individual has been diagnosed with an HIV infection or is considered to be at higher risk than the general population of becoming infected with HIV.

Aspect 51. The method of aspect 50, further comprising administering to the individual an effective amount of an agent that reactivates latent HIV integrated into the genome of a cell infected with HIV.

Aspect 52. An isolated biological fluid comprising the construct of any one of aspects 1-37.

Aspect 53. The isolated biological fluid of aspect 52, wherein the biological fluid is blood or plasma.

Aspect 54. A method of generating a variant interfering, conditionally replicating, human immunodeficiency virus (HIV) construct, the method comprising:
a) introducing the construct of any one of aspects 1-37 into a first individual;
b) obtaining a biological sample from a second individual to whom the construct of any one of aspects 1-37 has been transmitted from the first individual, wherein the construct present in the second individual is a variant of the construct of any one of aspects 1-37; and
c) cloning the variant construct from the second individual.

Aspect 55. An isolated cell comprising the construct of any one of aspects 1-37.

Aspect 56. A method of generating a particle, the method comprising transfecting a cell infected with a human immunodeficiency virus with the construct of any one of aspects 1-37, and incubating the cell under conditions suitable for packaging the construct in the particle.

Aspect 57. An isolated cell comprising the particle of any one of aspects 41-44.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s): s or sec, second(s); min, minute(s); h or hr, hour(s); aa, amino acid(s); kb, kilobase(s); bp, base pair(s); nt, nucleotide(s): i.m., intramuscular(ly); i.p., intraperitoneal(ly): s.c., subcutaneous(ly); and the like.

### Example 1: A Transmissible Antiviral Therapy for HIV: Discovery and Validation of the First Therapeutic Interfering Particles

Introduction

Current antiretroviral therapies (ART) are pharmaceutical-based and require lifelong administration—which is dependent on patient behavior/adherence—and interruption of therapy leads to viral rebound from latent reservoirs, clinical progression, and the threat of viral mutational escape (Metzger, et al., 2011; Notton, et al., 2014).

We have forwarded the idea that the historical concept of Defective Interfering Particles (DIPs), (Henle and Henle, 1943; Huang and Baltimore, 1970), could be rationally engineered into Therapeutic Interfering Particles (TIPs) that act as single-administration therapies with a high genetic barrier to resistance (Metzger, et al., 2011). Like DIPs, TIPs are sub-genomic deletion mutants, but TIPs are engineered to have a basic reproductive ratio (R₀)>1 (Metzger, et al., 2011; Rouzine and Weinberger, 2013).

For HIV-1 (hereafter “HIV”), TIPs were predicted to act as molecular parasites to deprive HIV of critical replication machinery while conditionally replicating along with HIV to reduce HIV burst size (FIG. 1). At the molecular scale, TIPs were predicted to lack the structural and envelope genes required to self-replicate (i.e., trans-acting elements) but retain the genetic signals for packaging and encapsidation (i.e., cis-acting elements). Thus, TIPs would co-opt HIV trans elements (e.g., capsid, envelope), using HIV as a helper virus to mobilize from infected cells. However, no evidence for a TIP existed.

Epidemiological models projected that TIPs-if they could be successfully engineered-would have the potential to significantly outperform existing and proposed interventions (FIG. 2). Specifically, TIPs could significantly reduce HIV set-point viral load in vivo (Weinberger, et al., 2003), leading to slower clinical progression and lower HIV transmission (Metzger, et al., 2011), and minimizing HIV evolution and mutational escape in patients (Rouzine and Weinberger, 2013) and in high-risk populations (Rast, et al., 2016). TIPs would achieve these benefits by reducing HIV set-point viral load-which remains a strong predictor of clinical progression (Mellors, et al., 1996) and viral transmission (Fraser, et al., 2007) despite other metrics (e.g., CD4⁺ cell count).

Mathematical models of HIV in vivo dynamics are well-established (Nowak and May, 2000) and laid the scientific premise for TIPs: the models showed that despite only ˜1% of cells being productively HIV infected, the vast majority of these cells become HIV-TIP co-infected (Weinberger, et al., 2003). The reason is the rapid turnover of HIV-infected cells: infected cells die in ˜1 day (Perelson, et al., 1997) such that a new subset of 1% of cells is infected each day during chronic set-point infection. As a consequence of these in vivo dynamics, the models showed that CD4⁺ T cells harboring integrated TIP proviruses become super-infected and transactivated by HIV within 30-50 days [quicker if during acute infection when a larger fraction of cells is HIV infected (Metzger, et al., 2011)]. Incidentally, this is similar to the conventional explanation for why HIV recombination is rapid—as recombination requires heterodiploid virions that are produced from cells super-infected with two distinct proviruses—despite cells with multiple HIV proviruses being rare (Josefsson, et al., 2011).

TIPs could Constitute ‘Salvage Therapies’ that Limit Viral Rebound Upon ART Interruption.

HIV-1 TIPs (and DIPs) are lentiviral vectors lacking Tat—and the associated positive-feedback circuit that controls latency. We, and others, have shown that such vectors obligately enter proviral latency and remain dormant until Tat is provided in trans (Razooky, et al., 2015; Weinberger, et al., 2005). Hence, during ART interruption or failure, latent DIPs/TIPs would be reactivated when HIV reverses latency or infects a transduced cell. TIPs could thus act as a salvage therapy—complementary to ART—to reduce HIV viral load during ART interruption (Metzger, et al., 2011). In fact, analytical therapy interruptions (ATIs) are a likely approach for testing, and eventually implementing, TIPs in the clinic (e.g., NCT03617198 a 24-week ATI clinical trial currently underway).

TIPs have a High Genetic Barrier to Mutational Escape

Mathematical models of HIV-1 in vivo dynamics indicate that DIPs and TIPs are intrinsically less vulnerable to HIV mutational escape than traditional therapeutics as they mutate using the same reverse transcriptase machinery, with a larger burst size, and are under evolutionary selection to maintain their parasitic relationship with wild-type virus. (Rast, et al., 2016; Rouzine and Weinberger, 2013). Preliminary models show that, overwhelmingly, the most likely mutations are TIP loss-of-function events (i.e., for the TIP to lose its therapeutic effect on wild-type HIV). Strikingly, in our models, even if TIP loss-of-function occurs twice as fast as HIV escapes from current ART, TIPs would still stably reduce AIDS population prevalence >10-fold better than potential vaccines (Metzger, et al., 2011).

Another concern is that TIPs may acquire via recombination an element that ‘upregulates’ pathogen production and in turn upregulates its own production from the cell. We explored the evolutionary selection aspects of this concern in detail (Metzger, et al., 2011; Rast, et al., 2016; Rouzine and Weinberger, 2013); strikingly, in these models, TIP-mediated upregulation of HIV led to increased TIP viral loads in patients, which actually generated even lower HIV viral loads and HIV population prevalence. Thus. TIPs are subject to competing selection pressures at multiple scales, which limits the potential evolutionary breakdown of these therapies.

Epidemiological Projections: TIPs could Circumvent Behavioral Barriers and Target High-Risk Groups.

Epidemiologists have long recognized the immense potential of targeting high-risk ‘core-groups’ and viral ‘superspreaders’ for efficient control of infectious diseases. However, it is often very difficult to identify and reach these high-risk populations: a problem further aggravated by nonhealthy-seeking behaviors in the key populations (e.g., injecting drug users, IDUs), and social stigma or criminal barriers that motivate high-risk individuals to remain unknown (e.g., IDUs, commercial sex workers and their clients, men who have sex with men, and people with extra-marital sexual partners). The resulting cost and effort involved in identifying high-risk populations have meant that-despite the huge potential benefits-targeting of disease-control measures is often not feasible in practice. TIPs surmount this obstacle by exploiting the underlying transmission patterns of disease spread to reach these key infectious populations via the same mechanisms and risk factors as the pathogen (Metzger, et al., 2011; Notton, et al., 2014). That is, TIPs are projected to yield the benefits of targeted control without the costs and challenges of identifying and reaching high-risk individuals. Current prevention and treatment approaches face the challenges of poor adherence and behavioral disinhibition—wherein successful disease control leads to a reduced sense of personal risk from the disease and can result in increases in risk behavior. Disinhibition remains a concern for HIV prevention and control (Dukers, et al., 2001) and can generate the perverse outcome wherein a successful therapeutic actually increases HIV incidence (Blower, et al., 2000). Single-administration therapies could effectively circumvent these problems, unlike ART or potential vaccines, and the public health benefits of TIPs are uniquely robust to disinhibition (Metzger, et al., 2011).

Genotoxicity: Lentiviral Vector Therapeutic Successes (e.g. CAR-T Cells) have Reduced Genotoxicity Concerns

Understandable concerns persist for retroviral integration due to early reports of insertional mutagenesis leading to oncogenic transformation (Pike-Overzet, et al., 2007). However, unlike that trial, TIPs do not depend on transducing hematopoietic stem cells (or any progenitor cell), but only on transducing terminally differentiated T cells and possibly macrophage—gene therapies into blood cells are now a successful therapeutic strategy as evidenced by CAR T therapies and other approaches. This could be due to the resistance of T lymphocytes to oncogenic transformation; T-cell leukemias are exceptionally rare, even in the HIV-infected population, and B-cell leukemias (more common in HIV patients), are not due to HIV integration in these cells.

Regardless, modern lentiviral therapies have strong safety records, consistently demonstrating a lack of insertion-mediated genotoxicity (Aiuti, et al., 2013; Biffi, et al., 2013; Hacein-Bey Abina, et al., 2015; Levine, et al., 2006; Malech and Ochs, 2015: Scholler, et al., 2012). Notably, retroviral vectors in T cells (CAR T cells) now have decade-long safety and efficacy profiles (Scholler, et al., 2012) and recent success in sickle-cell disease (Ribeil, et al., 2017) and SCID-X1 infants (Mamcarz, et al., 2019). Thus, insertional genotoxicity and oncogenesis of lentiviral vectors (e.g., DIPs and TIPs) appear minimal.

Recombination: Lentiviral Vectors do not Exhibit Recombination with Wild-Type HIV in Cell-Culture, Mice, or Humans.

Being pseudo-diploid, lentiviruses carry two genomic RNA strands that can recombine during reverse transcription. Lentiviral recombination appears to require significant homology in sequence, and recombination between highly heterozygous gRNA strands, such as one full-length 9.7 kb HIV gRNA and a significantly shortened gRNA of 3-7 kb, fail to produce viral progeny due to a block prior to provirus integration (An, et al., 1999). This molecular finding is in agreement with data from murine models (Davis, et al., 2004) and human clinical trials (Levine, et al., 2006), neither of which detected recombination between wild-type HIV and shorter lentiviral vectors. Our preliminary data (below) also fail to show recombination between our putative TIP, F1, and HIV.

### Discovery of the F1 DIP

TIPs have the potential to act as single-administration therapeutics with a high genetic barrier to resistance (Metzger, et al., 2011; Rast, et al., 2016; Rouzine and Weinberger, 2013; Weinberger, et al., 2003). Given the potential benefits of TIP therapy, we set out to identify candidates for HIV TIPs. Ideally, we wanted to modify an existing DIP to meet the criteria outlined above (i.e. high transmission and a one-log reduction in viral outgrowth) but the HIV “DIPs” described in the literature fail to transmit.

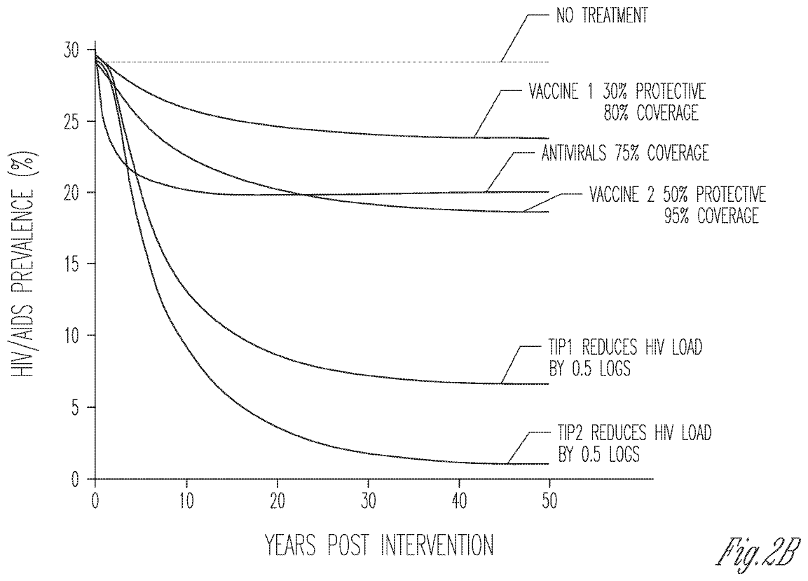

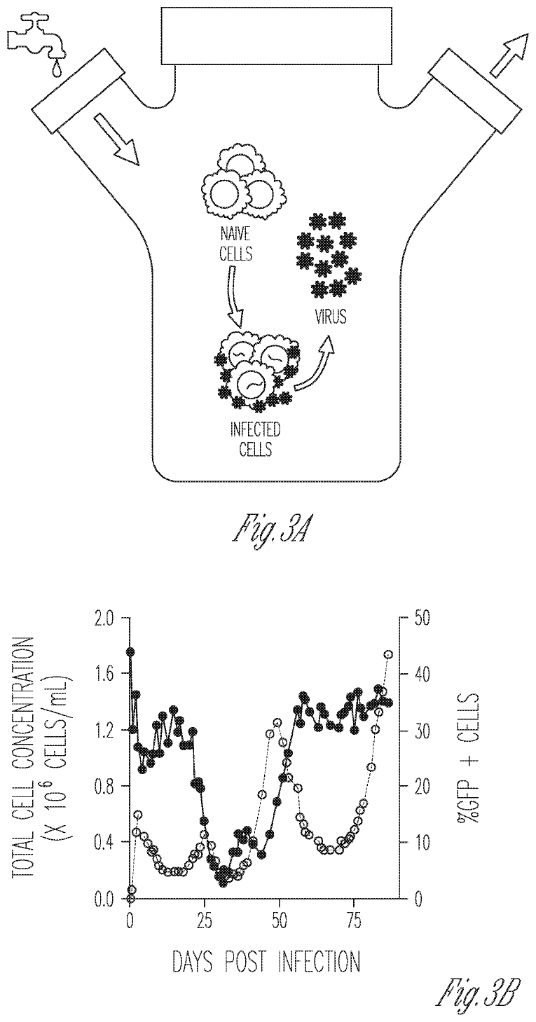

Based on models of HIV that a DIP may take ˜50 days to reach detectable levels (Weinberger, et al., 2003), we constructed a continuous reactor system to culture HIV long term in CEM T cells (FIG. 3). The reactor generated von Magnus-like oscillations in infected cell numbers (FIG. 3) and we detected a ˜2.5 kb deletion (designated F1 for flask 1) in the cells that survived. Notably, the F1 deletion arose repeatedly (i.e., in five subsequent experiments) and has not appeared in previously sequenced patient samples (Bruner, et al., 2016; Cohn, et al., 2015; Ho, et al., 2013; Pollack, et al., 2017) and our screen, described below (FIGS. 4-6), provides a potential explanation.

Engineering the HIV F1 DIP into a TIP

Preliminary data indicated that while the F1 DIP interfered with HIV, it failed to be efficiently mobilized by HIV in trans (i.e., its R₀<1). Therefore, further engineering of F1 was required to meet the criteria for a TIP. To engineer F1 into a TIP we utilized two approaches: (i) screening a random-deletion library, and (ii) splicing ablation.

i) Random library screen. To systematically identify regions of HIV required for efficient mobilization, we utilized a randomized deletion screen (Weinberger and Notton, 2017) to create & index random-deletion libraries of HIV NL4-3 (FIG. 4). Briefly, plasmid DNA was subjected to transposon-mediated random insertion, followed by excision of the transposon and exonuclease-mediated digestion of the exposed ends to create deletions centered at a random genetic position, each of variable size. The plasmid is then re-ligated together with a cassette containing a 20-nucleotide random DNA barcode to ‘index’ the deletion. Indexing is critical as it allows a deleted region to be easily identified (by the junction of genomic sequence and the barcode) and tracked/quantified by deep sequencing.

The randomized, barcoded library yielded ˜120,000 variants (a 15% subset of the full library shown in FIG. 5). The deletions in the pNL4-3 plasmid, occur across the entire plasmid except at the origin of replication/beta-lactamase in the plasmid, which is expected given that deletion of these regions is strongly selected against in bacteria (these regions are natural controls for the experiment). The mean deletion size was 1.4 kb but ranged up to 6 kb (Weinberger and Notton, 2017).

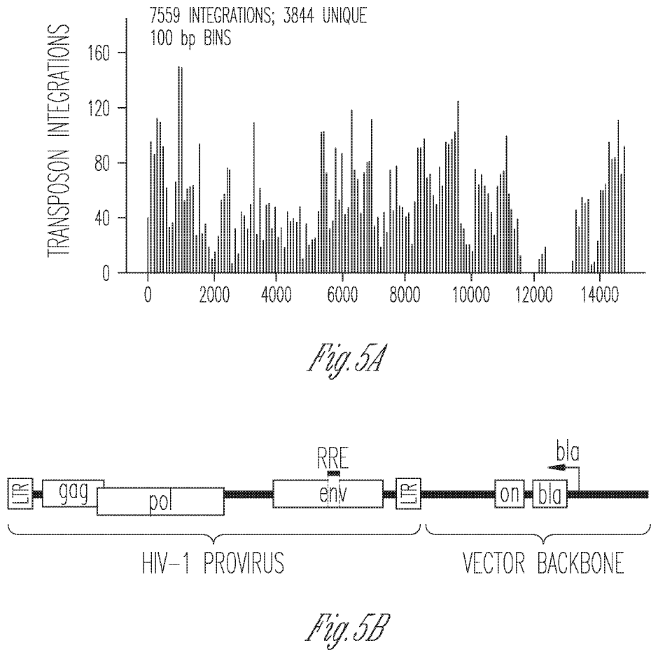

Screening of the library was carried out by repeated high-MOI (MOI=5) passage in vitro in the presence of replication-competent HIV acting as a helper virus (i.e., the classical DIP-isolation approach for RNA viruses). As in previous approaches. MOI=5 was used to ensure that defective mutants with the potential to be complemented in trans would have the opportunity to amplify upon super-infection by replication-competent HIV (replication-competent viruses are also selected but subsequently filtered out). Barcodes were analyzed by deep sequencing after 12 passages (FIG. 6A). Briefly, the barcoded deletion library was packaged into viral-like lentiviral particles in 293T cells and used to transduce a T-cell line (MT-4). MT-4 cells were subsequently passaged 12 times at high MOI (MOI>5) through both cell-to-cell and cell-free transfers. Viral RNA was isolated from the supernatant at periodic time points and deep sequenced via Illumina sequencing of the barcode cassettes, allowing computation of a deletion-depth landscape after each passage. The frequency at which each base is spanned by a barcoded deletion is given by the “deletion depth”, analogous to “read depth” in the DNA deep-sequencing literature. Between passages 9-12, the deletion landscape stabilized allowing generation of a map—at single-base resolution—of HIV genome regions intolerant to deletion (cis-acting elements) and regions tolerant to deletion (trans-acting elements) (FIG. 6B) (Weinberger and Notton, 2017). The landscape recapitulated known cis-acting elements (e.g., RRE, Ψ, cPPT/CTS, etc.) with the cPPT/CTS being most striking as it is located directly within the F1 deletion that spans pol through vpr (FIG. 3). The cPPT/CTS element is known to aid nuclear import (Dardalhon, et al., 2001) and hence, we reinserted it into the F1 deletion to generate F1^{cPPT}, which we hypothesized might constitute a TIP.

ii) Ablation of HIV splicing. Next, to enhance F1 gRNA production, we built off our recent mapping of HIV's regulatory circuit (Hansen, et al., 2018a) showing that HIV splicing is obligately post-transcriptional (FIG. 7). We mutated splice-acceptor sites in F1^{cPPT}, to limit F1 gRNA splicing and generate only gRNA. All F1^{cPPT} P vectors mentioned hereafter encode the mutated splice-acceptor sites. By increasing the F1^{cPPT} gRNA intracellular abundance relative to HIV gRNA, which is spliced, we hypothesized that F1^{cPPT} would package more efficiently.

The F1^{cPPT} recombinant. The F1^{cPPT} recombinant (FIG. 8A) was trans-complemented by HIV (FIG. 8B), and, strikingly, elicited a 1-Log reduction in HIV infectious titer (FIG. 8C) RT-qPCR analysis of cytoplasmic and supernatant gRNAs showed that F1^{cPPT} packaged more of its gRNA into virion particles than HIV (FIG. 8D).

F1^{cPPT} requires HIV to mobilize to new cells. To quantify mobilization efficiency, we developed a co-culture assay. Naïve CEM target cells, stably expressing an mCherry reporter, were co-cultured together with cells pre-transduced with F1^{cPPT} (which carries a GFP reporter). After 2 days of co-culture, we observed a large fraction of dual-positive (mCherry+GFP+) cells, but only in the presence of HIV infection (FIG. 9).

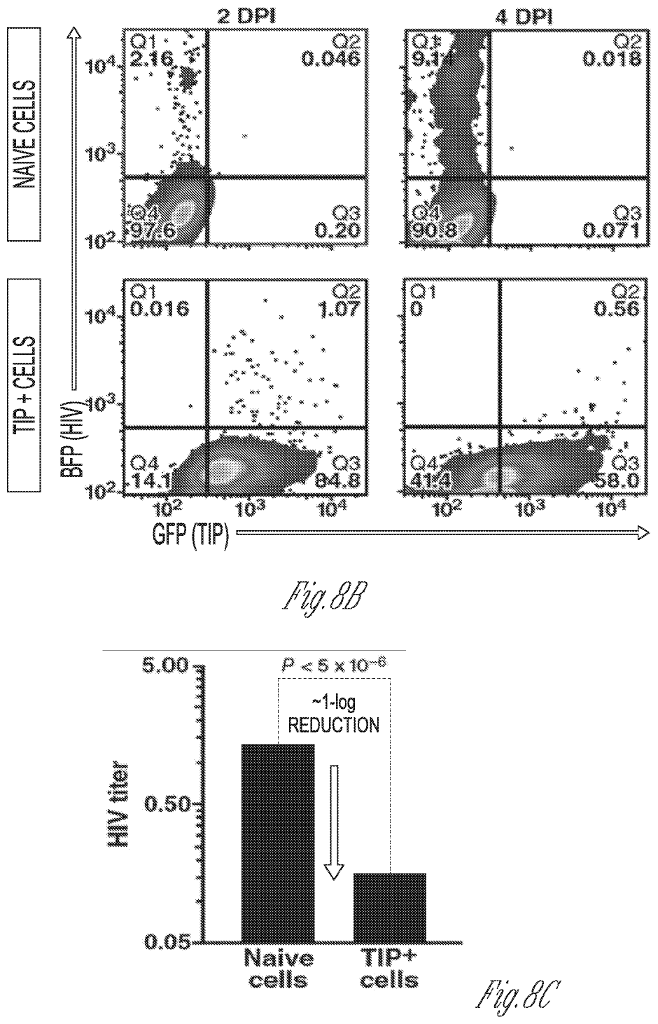

F1^{cPPT} exhibits R₀>1 (satisfying criterion for a TIP). To determine the R₀ of F1^{cPPT}, we quantified spread of F1^{cPPT} (by GFP) into the mCherry target cells. Briefly, cells transduced with F1^{cPPT} were infected with HIV-BFP and then diluted 10:90 in naïve mCherry-expressing cells. This was done since the formal definition of R₀ holds only when the vast majority of cells are susceptible to infection (i.e., initial infection). In parallel, we also measured the kinetics of the rate of F1^{cPPT} spread (by GFP) through mCherry cells. Both, approaches gave measures of R₀>1 for F1^{cPPT} and showed that the R₀ for HIV was reduced in the presence of F1^{cPPT}(FIG. 10).

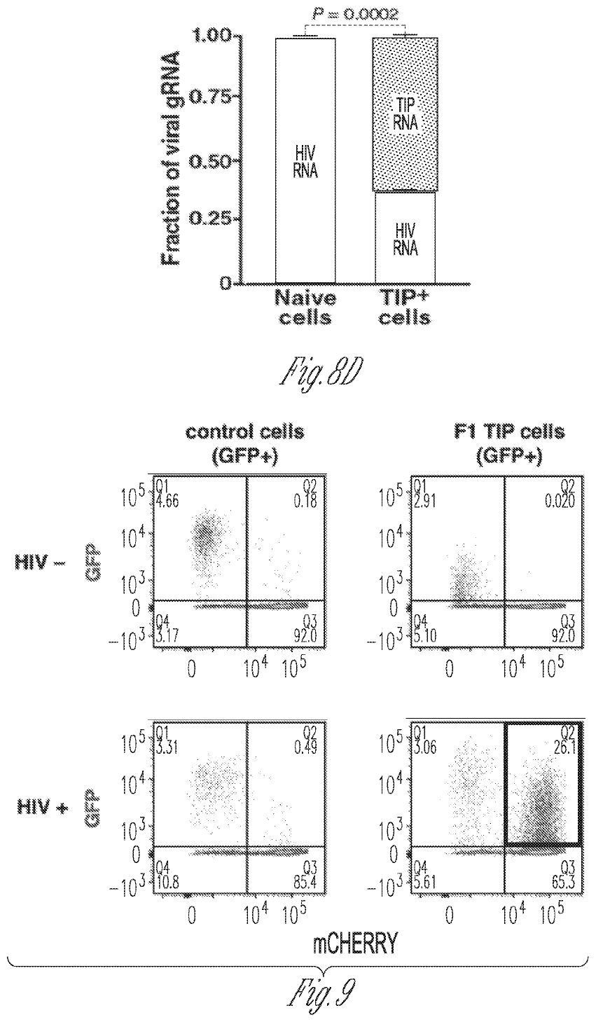

F1^{cPPT} efficiently interferes with HIV in long-term culture and exhibits a possible signature of co-evolution. To determine if F1^{cPPT} could inhibit HIV in a multi-round, spreading infection, we used the CEM T-cell reactor approach (FIG. 3). The reactor was seeded with a fraction of cells pre-transduced with F1^{cPPT}. At 1:1 seeding with F1^{cPPT} cells, there was a substantial inhibition of HIV spread through the reactor (FIG. 11) and F1^{cPPT} mobilized to >50% of cells. Seeding the reactor with 5% and 10% F1^{cPPT} T also exhibited interference with HIV, but theory (Rouzine and Weinberger, 2013; Weinberger, et al., 2003) predicts that 50-100 days would be required to see full inhibition at such seeding fractions. Surprisingly, around day 25, the F1^{cPPT} reactor exhibited a significant increase in HIV infected cells followed 10 days later by a drop. These types of dynamic ‘blips’ are a canonical signature of co-evolutionary arms-races between bacteriophages and E. coli (Lenski and Levin, 1985).

F1 interferes via multiple mechanisms of action. To address how F1 interferes with HIV while also enhancing encapsidation of its own gRNA, we initiated a set of biochemical and mutational studies.

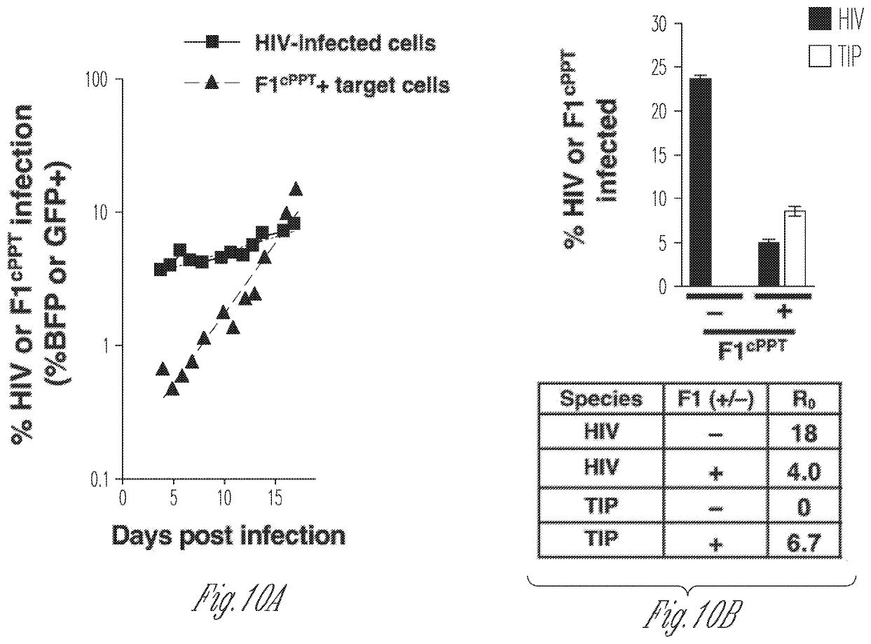

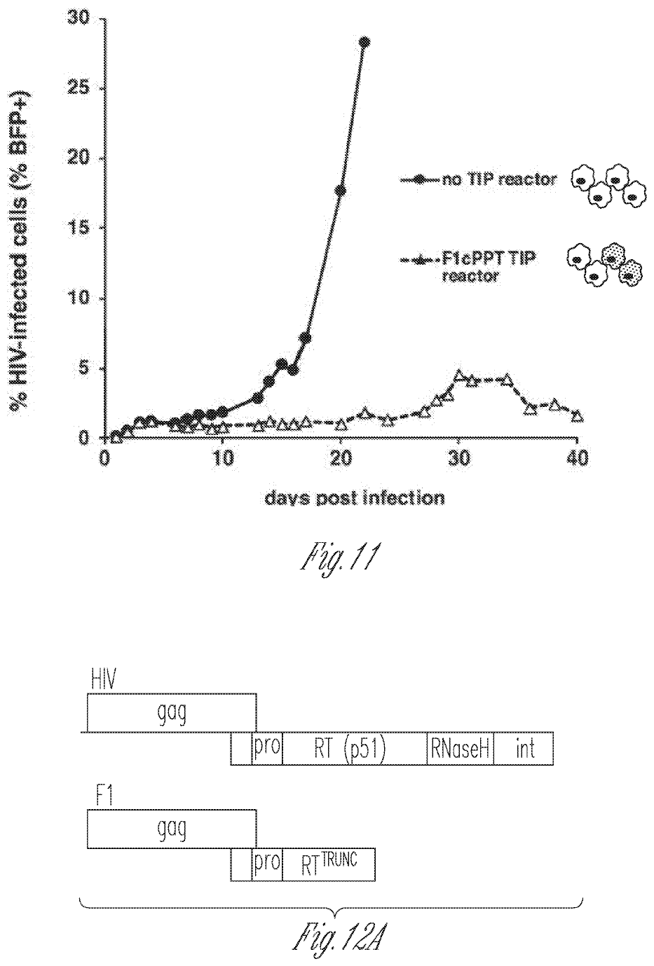

F1 truncation protein is required; full RT deletion does not recapitulate phenotype. Since the F1 deletion truncated the reverse transcriptase (RT) gene (FIG. 12A), we first analyzed RT activity in viral supernatants with and without F1. As expected, RT activity was significantly reduced in the F1 supernatants (FIG. 12B). However, when RT was fully deleted from F1 (F1ΔRT; expansion of the F1 deletion) only partial HIV interference occurred (FIG. 12C). These data indicate that RT does not fully account for the interference phenotype and that F1 appears to act, in part, as a dominant negative protein.

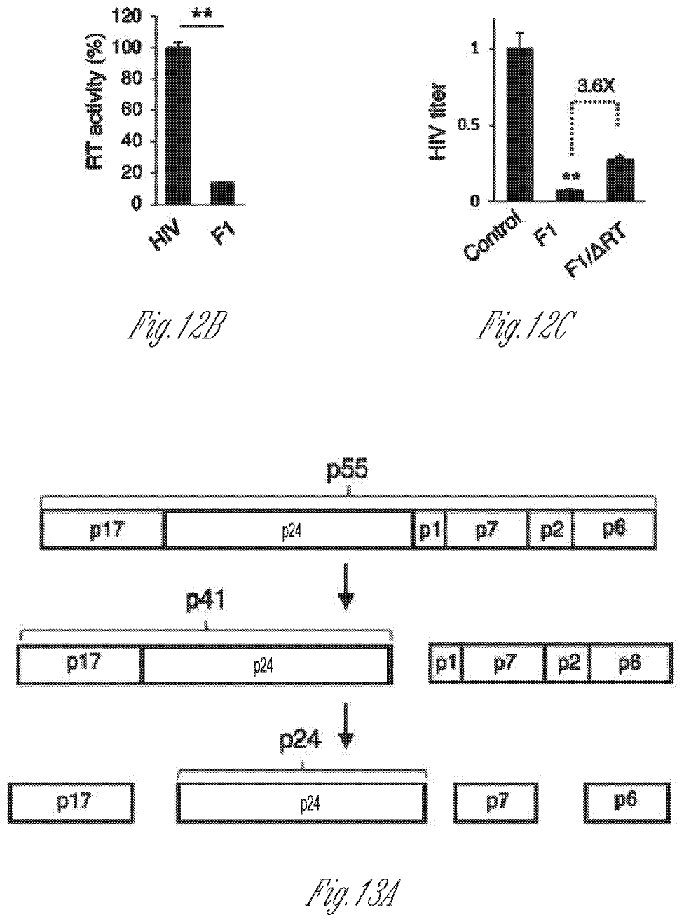

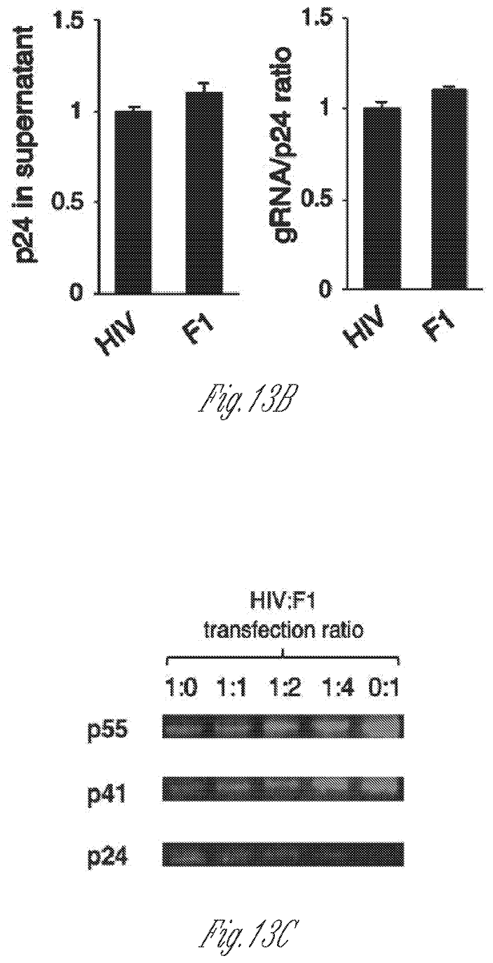

F1 interferes with Gag processing. Since certain non-nucleoside RT inhibitors (e.g., efavirenz) misregulate Gag-Pol polyprotein processing (Figueiredo, et al., 2006), we tested if F1 also disrupted Gag-Pol processing (FIG. 13). Despite F1 cultures producing similar levels of p24 capsid protein in the supernatant and exhibiting similar gRNA:p24 ratios (FIG. 13B), Western blot analysis showed a severe defect in Gag proteolytic cleavage (FIG. 13C), which is consistent with Gag processing defects being potent dominant negatives (Lee, et al., 2009). A notable increase in overall Gag polyprotein translation was also observed in the presence of F1 (FIG. 13C) and will be further explored by ribosomal fractionation.

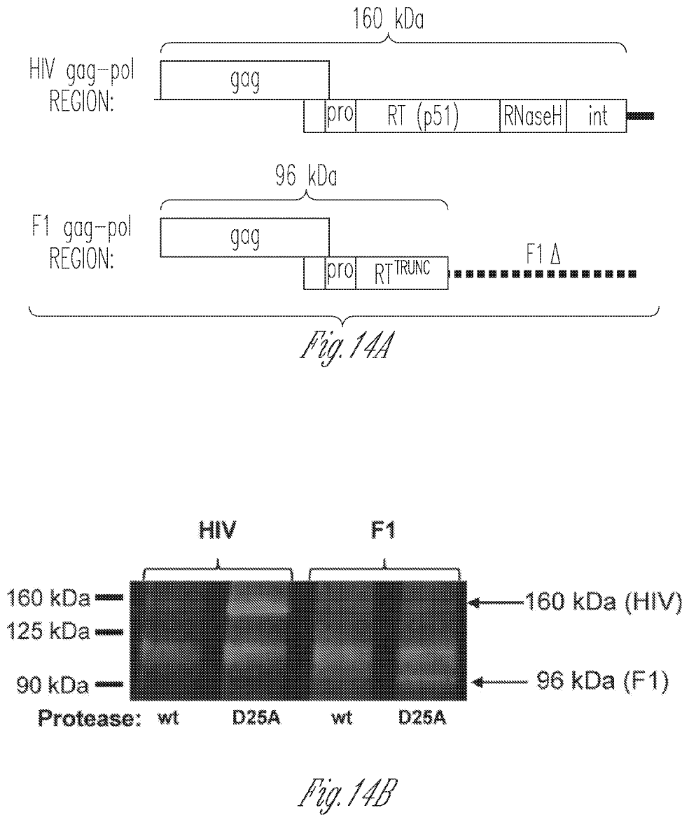

The F1 truncation protein is packaged in virions. We next developed an assay to test if truncated F1 proteins were packaged into virion particles. Western blot analysis of virion particles indicated that the truncated protein is present in virion particles (FIG. 14).

### Electron Microscopy of F1 and HIV Virion Particles

‘Hybrid’ HIV-F1^{cPPT} virion particles are being characterized by electron microscopy in the Sundquist laboratory (Univ. of Utah) to determine whether incorporation of variant F1 proteins and RNA genomes influences virion ultrastructure, and to define which stage(s) of virion morphogenesis are altered.

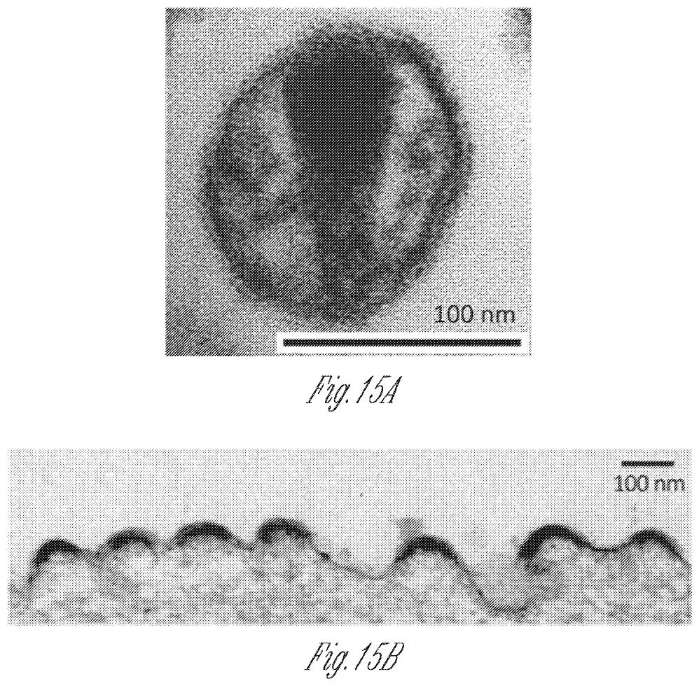

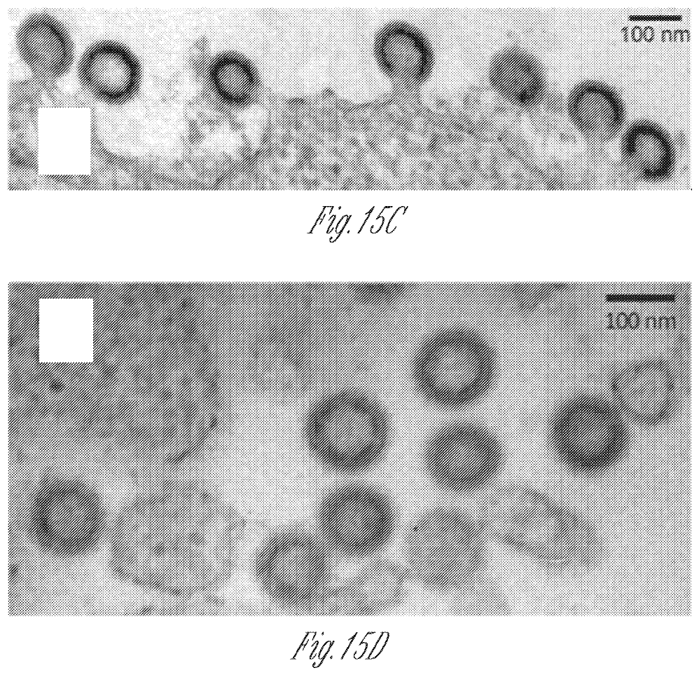

During normal HIV virion morphogenesis, the viral Gag polyprotein assembles at the plasma membrane, becomes enveloped in the membrane, buds from the cell, and then assembles a mature conical viral capsid following Gag processing (Sundquist and Krausslich, 2012). Mature infectious HIV particles therefore contain central conical capsids that are visible in electron micrographs of thin sectioned virions (FIG. 15A). Virions that fail to reach this mature, infectious state can arrest at different stage of biogenesis, and the Sundquist laboratory has previously characterized HIV virions that (i) failed during envelopment (FIG. 15B) (Mercenne, et al., 2015), (ii) budding (FIG. 15C) (Garrus, et al., 2001), and (iii) maturation (FIG. 15D) (Hiatt, et al., 2019), owing to aberrant interactions with different essential host factors.

The morphologies of thin-sectioned HIV virions produced in the presence of F1^{cPPT} are examined using staining protocols designed to optimize capsid visualization (Jurado, et al., 2013). As controls, wild-type HIV particles and pure F1^{cPPT} virus-like particles (VLPs) are examined in parallel, and all virion phenotypes are scored quantitatively (Hiatt, et al., 2019; Mercenne, et al., 2015). If morphological defects are observed in thin sectioned F1^{cPPT} DIP/HIV hybrid particles, these virions are imaged at higher resolution and in their native states by cryo-EM, both in projection images, and by electron cryotomography (ECT). The latter approach allows us to visualize the 3D spatial characteristics of assembly and maturation defects. We have previously performed ECT reconstructions to characterize mature (Benjamin, et al., 2005) and immature (Wright, et al., 2007) HIV particles (with Jensen), and designed enveloped protein nanoparticles (Votteler, et al., 2016) (ourselves). Approaches for reconstructing HIV virions have advanced rapidly (Hagen, et al., 2016; Mattei, et al., 2016; Schur, et al., 2016), and we can now produce the highest possible resolution reconstructions using a Titan Krios microscope, with a K2 detector and energy filter. SerialEM (Mastronarde, 2005) is used to automate tilt series acquisition using the low dose mode, tomograms are generated and processed using the IMOD software package (Kremer, et al., 1996), and segmentation and rendering is performed in Amira (FEI). Collectively, these experiments show whether co-assembly of F1 truncation proteins with wild-type HIV Gag alters particle morphology, define the stage(s) at which any aberrations occur, and visualize their structures at the highest possible resolution.

### Mass Spectrometry Analysis of HIV and F1 Virion Proteomes

To assess if F1^{cPPT} interferes by altering the protein composition or polyprotein processing of virions, the protein composition and processing status of virions are measured together with Dr. Nevan Krogan (Gladstone/UCSF) using a mass spectrometry-based proteomics approach.

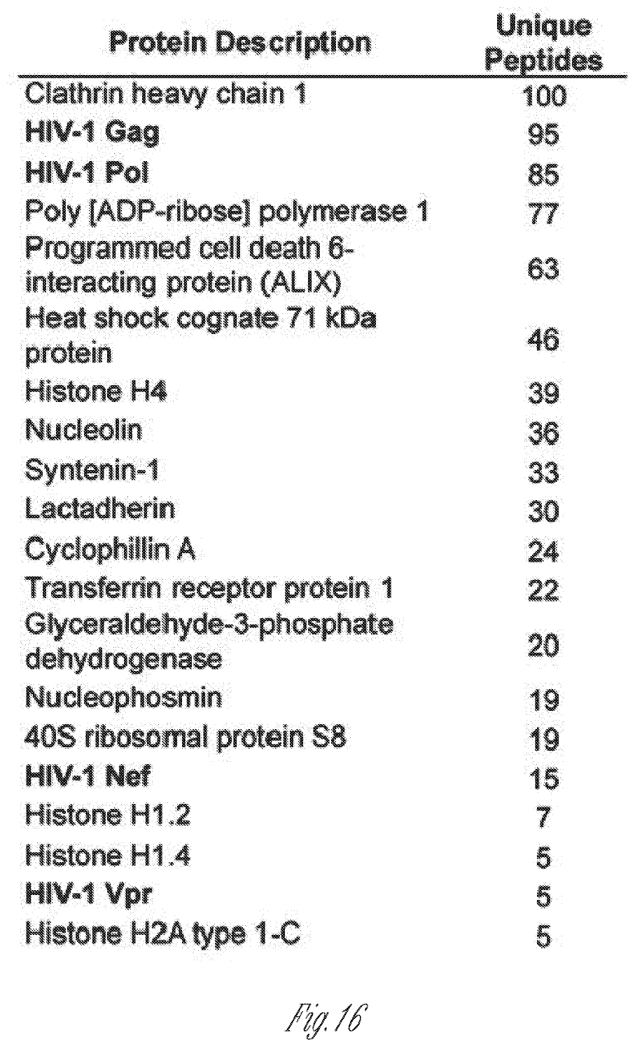

The Krogan lab (UCSF) has developed mass spectrometry proteomics approaches to determine HIV protein-protein interaction networks (Chou, et al., 2013; Jager, et al., 2011a; Jager, et al., 2011b) and recently applied LC-MS/MS mass spectrometry to assay protein composition of virions (FIG. 16). The analysis identified the expected viral components (Gag, Pol, Vpr, and Nef) and host components (e.g., ALIX, cyclophillin A, and clathrin) packaged in the virions. HIV Gag peptides represented >85% of the primary sequence, including peptides that span most cleavage sites.

We are applying a similar approach to quantify differences in protein composition and protein processing status for HIV virions produced in the presence and absence of F1^{cPPT}. HEK293T cells will be used to package pure F1^{cPPT}, or co-package HIV and F1^{cPPT} hybrids (empty plasmids will be used to subtract non-virion proteins enriched by the purification procedure). All experiments are performed in biological triplicate. Purified virions or VLPs are normalized by p24 using a FLAQ assay, digested with trypsin, and analyzed by LC-MS/MS on an Orbitrap Fusion MS system. As in our previous studies, peptide sequences are matched to raw mass spectra, intensities extracted by MaxQuant, and statistical analysis performed using the MSstats package to identify proteins and cleavage site-spanning peptides that are differentially abundant in virions produced in the presence vs. absence of F1^{cPPT}.

### Single Virion Imaging of F1 RNA Packaging

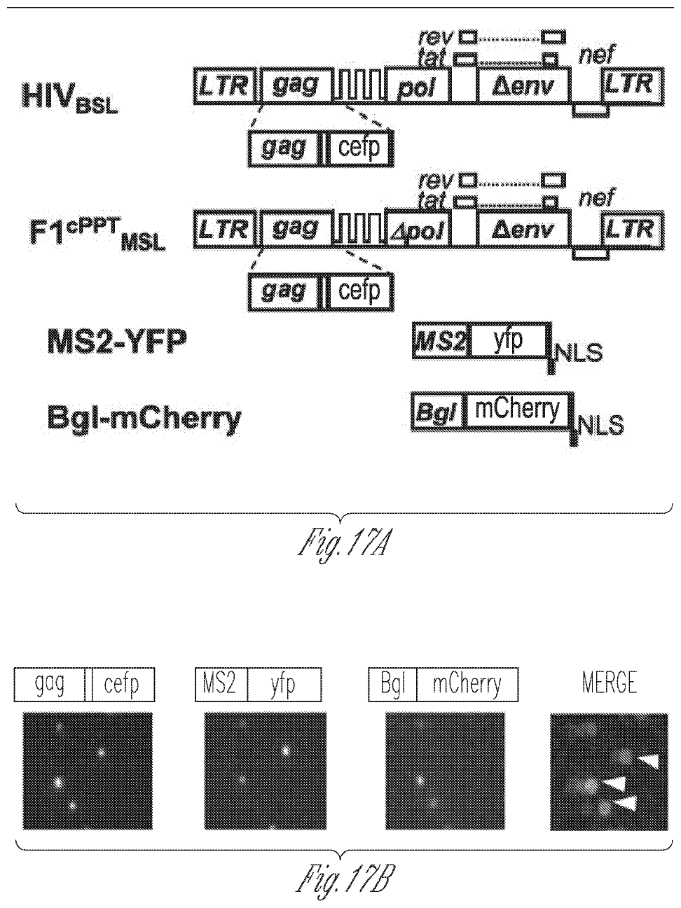

The effect of F1^{cPPT} on RNA packaging into virions is measured by imaging-based single virion analysis (SVA) together with Dr. Wei-Shau Hu (NCI/NIH), who pioneered SVA (Chen, et al., 2009). The SVA imaging approach (FIG. 17) demonstrated that >90% of HIV virion particles package two viral RNAs (Chen, et al., 2009). More recently, we extended SVA to live-cell imaging using total internal reflection fluorescence (TIRF) microscopy to show: (i) HIV RNAs dimerize not in the cytoplasm but on the plasma membrane, (ii) stabilization of the RNA dimer requires Gag protein, (iii) dimerization occurs at an early stage of virus assembly, and (iv) that dimerization is mediated by the interactions of two RNA-Gag complexes, rather than two RNAs (Chen, et al., 2016).

SVA imaging is used to compare F1^{cPPT} RNA packaging to HIV RNA packaging in the presence and absence of F1 or F1^{cPPT}. As described (Chen, et al., 2009), SVA uses two fluorescent protein labels (FIG. 17A). HIV (NL43) and F1/F1^{cPPT} is re-cloned to encode either a set of Bg1 stem loops (BSL) or MS2 stem loops (MSL); BSL and MSL are recognized by their RNA-binding coat proteins Bg1 and MS2, respectively, and binding to its cognate stem loops catalyzes coat protein localization into punctate foci. When the Bg1 and MS2 coat proteins are fused to YFP and mCherry, RNAs can be visually tracked by following the YFP and mCherry puncta. This enables quantification of the fractions of: (i) virions with two HIV RNAs (homodiploid), (ii) virions with two F1^{cPPT} RNAs (homodiploid F1), and (iii) virions with one HIV and F1^{cPPT} RNA (heterodiploid).

In addition, Gag fusion to Cerulean fluorescent protein (CeFP)—both tagged and untagged Gag are used to ensure normal morphology of the viral particles—enables spatiotemporal analysis, by TIRF microscopy, of the intracellular dynamics of how HIV and F1^{cPPT} RNA localize with Gag, as described (Chen, et al., 2016). F1^{cPPT} RNA's translation efficiency is also assayed by polysome fractionation which the Hu lab recently adapted for HIV, in this way, we determine whether F1^{cPPT} is simply competing stoichiometrically for packaging with HIV RNA or if per-capita RNA affinity for Gag and capsid is enhanced.

### Quantify F1 Interference Across a Broad Spectrum of HIV Patient Isolates and Cells

Transformed cell lines and HIV lab strains are convenient tools but do not reflect the biological complexities of infection in vivo. Thus, the use of primary human and patient cells has become standard. Here, we focus on examining F1^{cPPT} efficacy in patient cells and virus isolates.

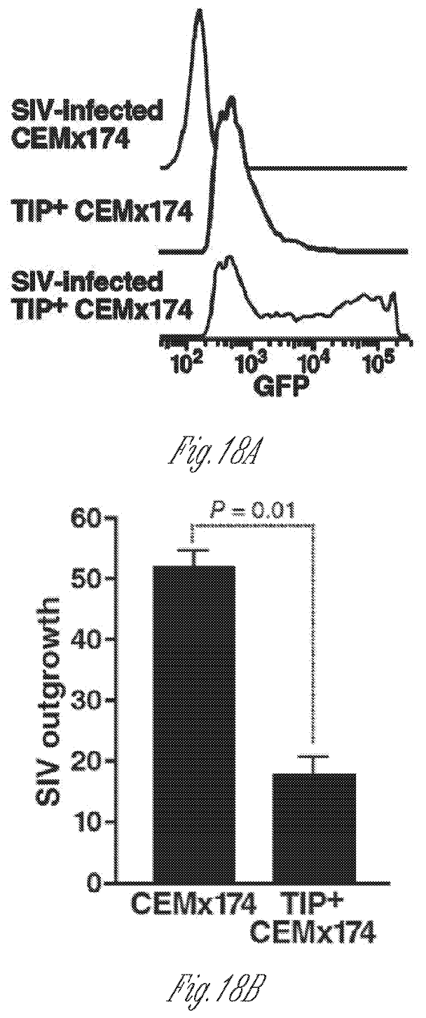

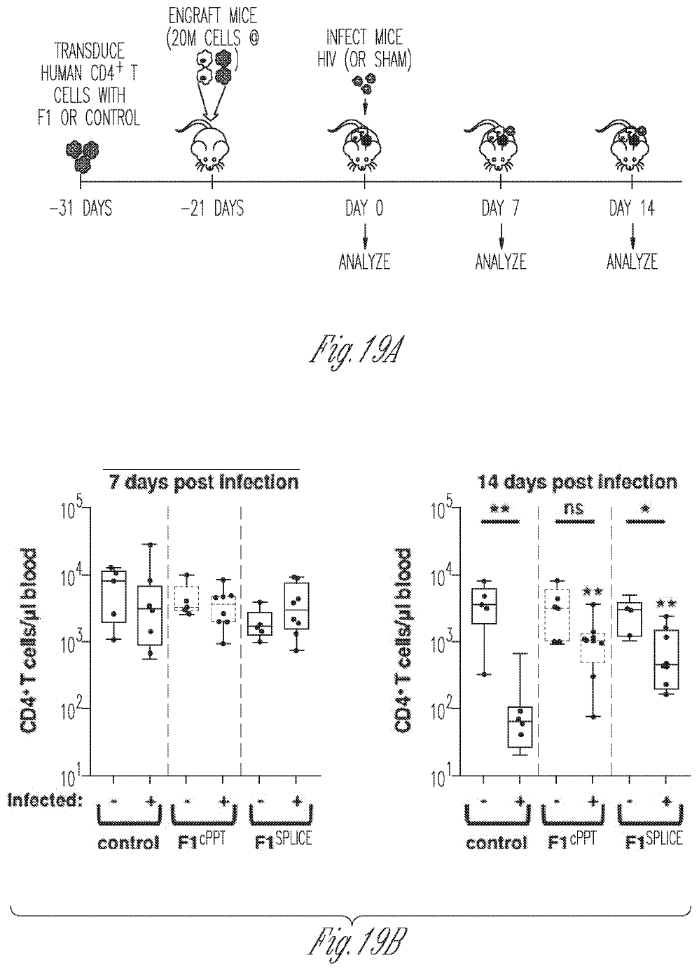

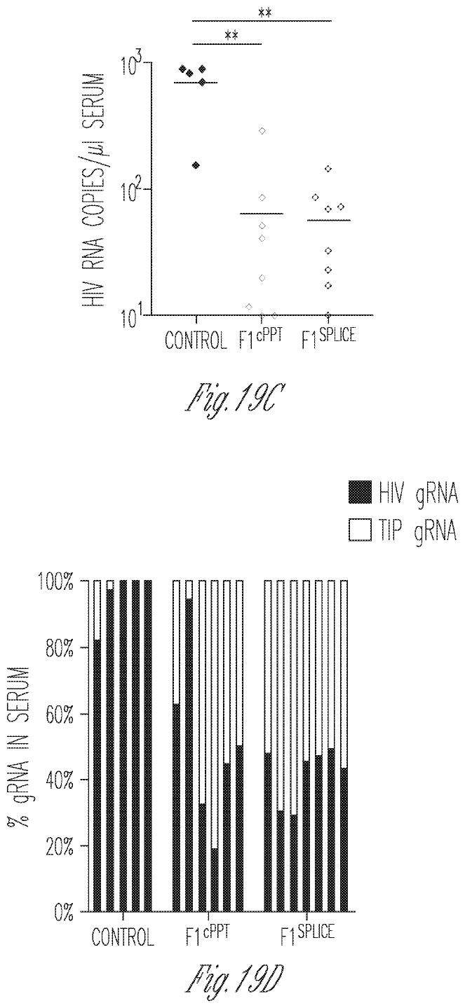

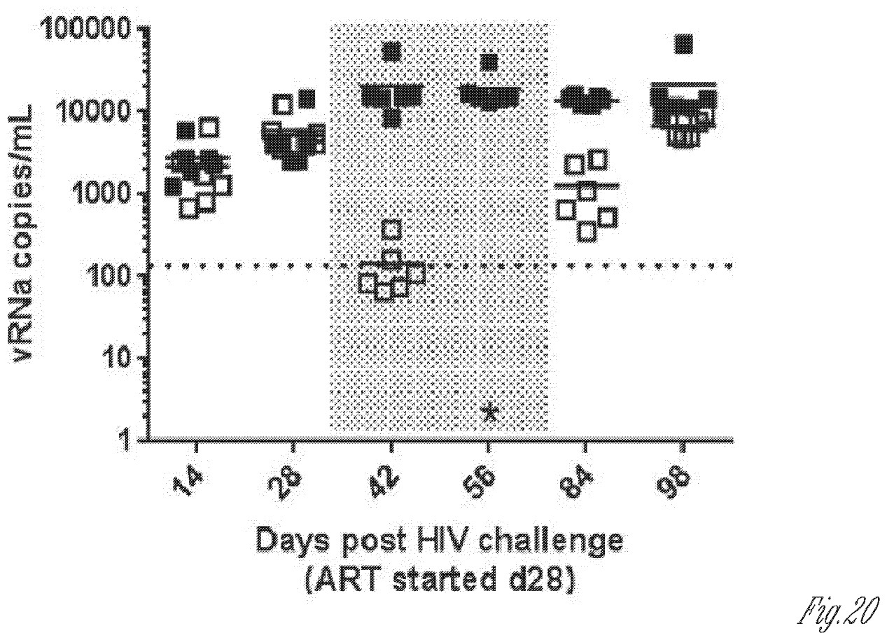

Building on our previous HIV studies in donor-derived αCD3/28-activated primary human CD4⁺ T cells (Hansen, et al., 2018a; Razooky, et al., 2015), we verified that the F1^{cPPT} TIP could interfere with HIV in human primary CD4⁺ T cells in vivo (initial experiments for mouse data in FIG. 19). Moreover, we found that F1^{cPPT} was trans-activated by the HIV-2-derived SIVmac239 molecular clone and significantly interfered with SIV in CEM×174 cells (FIG. 18). Given the sequence divergence between HIV-2 and HIV-1, F1's ability to interfere with HIV-2 argues it could interfere with a broad spectrum of HIV-1 subtypes.

F1^{cPPT} effect on diverse HIV patient virus isolates. Patient cells are obtained from the SCOPE cohort at UCSF (clinicaltrials.gov/ct2/show/NCT00187512). As we previously described, patient CD4⁺ T cells are isolated from large-volume (220 mL) blood draws (Eriksson, et al., 2013). To account for inter-patient variability, F1^{cPPT} are tested on ≥10 patient samples. Cells will be PMA/ionomycin activated, transduced with F1^{cPPT}, or sham vectors, and infected with 10 primary patient-virus isolates from a panel of international isolates (including subtypes A, B, C, D and circulating recombinants) obtained from the NIH AIDS reagent database (Cat #180130). Although cells from healthy donors could be used, testing on cells from infected patients accounts for potential physiological differences in the cells of chronically infected, ART-treated patients that may inhibit F1^{cPPT} function. For all isolates, HIV outgrowth from patient cells are quantified by a limiting-dilution outgrowth assay using probe-based qPCR (Hansen, et al., 2018a)—a slight modification of the standard QVOA which measures p24, since F1^{cPPT} virions also carry p24.

F1^{cPPT} effect on virus outgrowth from patient cells. HIV outgrowth from patient cells is directly quantified in the presence/absence of F1^{cPPT} transduction, as described (Eriksson, et al., 2013). In addition, historical, cryopreserved cell stocks from untreated patients (viral loads>10³ copies//mL) are F1P′ transduced, PMA/ionomycin activated, and outgrowth quantified as above. The Weinberger and Deeks labs have extensive experience in latency reversal and quantifying viral outgrowth (Dar, et al., 2014: Eriksson, et al., 2013). To account for inter-patient variability, ≥10 patient samples will be tested.

### Quantify F1 Interference Over Time and Co-Evolution in a Humanized Mouse Model

The long-term duration of F1^{cPPT} interference, co-evolutionary dynamics, and potential genotoxicity are assayed in the hu-HSPC NSG mouse model together with John Burnett (City of Hope) and Paula Cannon (Univ. of Southern California). Compared to the bone-marrow liver thymus (BLT) mouse (Denton, et al., 2012), which requires complicated surgical procedures for co-implantation of multiple tissue fragments, hu-HSPC NSG mice require no such surgery, enabling a larger number of animals to be studied per experiment (an important consideration for FDA approval).

With Dr. Jim Riley (Univ. of Penn.) we recently completed preliminary experiments in the HIV hu-PBL mouse model (FIG. 19a). F1^{cPPT} and a version with more extensive splicing ablation (F1^{splice}) both inhibited HIV by 1.5-2 Logs and protected CD4+ T cells by >1-Log (FIG. 19b-d). Similar results were observed in a second replicate experiment (not shown). However, hu-PBL is a short-term model: mice succumb to graft-vs-host disease (GVHD) in ˜5-6 weeks. The Burnett and Cannon labs established an in vivo model system for testing HIV cure strategies in HIV-challenged, humanized NOD.Cg-Prkdcscid IL2rgtm1Wjl/SzJ (NSG) mice (Akkina, et al., 2016; Holt, et al., 2010; Satheesan, et al., 2018). Critically, hu-NSG mice can recapitulate HIV latency and maintain viremia for ˜40 weeks without GVHD, establish relatively stable set-point viremia (FIG. 20), and Drs. Burnett and Weinberger have a history of collaboration (Weinberger, et al., 2005).

hu-HSPC NSG mouse studies: To test long-term F1 interference, hu-NSG mice are prepared as described (Ishikawa et al., 2005). Male and female mice are equally distributed among all experimental and control groups to account for any gender-biases in cell engraftment. At 12-14 weeks post engraftment, humanized mice are challenged with 100-200 ng p24 of HIV-1_{BaL} by IP injection. At 3-4 weeks post-infection, animals are in vivo transduced with gp120 env pseudo-typed F1^{cPPT} or sham vector via tail vein injection of 10⁸ IU. Initial transduction efficiency is assayed by FACS for GFP expression and a recent ddPCR assay, the intact provirus detection assay, IPDA (Bruner, et al., 2019), is adapted to quantify cells with intact F1^{cPPT} and HIV proviruses.

HIV viremia, cell counts, and cytokine profiles (via ELISpot/FluorSpot) are assayed weekly from peripheral blood (collected by retro-orbital bleed) in F1^{cPPT}- and sham-transduced mice. At week 40, mice are necropsied to harvest spleen, liver, bone marrow, intestine, and brain for analysis of HIV and F1^{cPPT} gDNA by ddPCR; we have found persistent HIV infection in these tissues (Satheesan, et al., 2018).

Co-evolution analysis via sequencing: Full-length gDNA collected from mouse blood/tissue is analyzed on a PacBio RSII Single Molecule, Real-Time (SMRT) Sequencing system (City of Hope Sequencing Core) for possible signatures of sequence co-evolution (e.g., dN/dS analysis of F1 and HIV Gag). This analysis determines if HIV sequences subsets are evolving more rapidly in the F1^{cPPT} treated animals and how or if F1^{cPPT} is diverging from its parent sequence. PacBio technology combines deep massive-parallel sequencing of NGS with long-sequencing reads (>10 kb), can simultaneously sequence mixtures of up to 40 different full-length HIV-1 genomes (Dilernia, et al., 2015). The Gladstone Genomics Core has extensive experience analyzing PacBio data (Thomas, et al., 2014) and will assist our efforts.

Analytical Therapy Interruption (ATI): To mimic the structure of potential human clinical trials (NCT03617198), ATI studies are performed on hu-NSG mice receiving ART. We have established a protocol for oral-delivery of ART to HIV-infected mice and demonstrated latency during ART (FIG. 20): undetectable HIV RNA in serum followed by robust and rapid viral rebound 2-4 weeks following ART interruption (Satheesan, et al., 2018). At 4 weeks after HIV challenge, mice are infused with F1^{cPPT} and then at 8 weeks ART is administered until viremia is undetectable. ART is interrupted 4-6 weeks later, and HIV vRNA is quantified until week 40 to determine if F1^{cPPT} delays or controls HIV rebound. At week 40, mice are necropsied for tissue analysis.

TABLE 1
F1pol

| | RETAINED | | | |
|---|---|---|---|---|
| | nt coordinates | | | |
| | in NL4-3 | | | |
| RETAINED | genome | | | |
| proteins | start | end | HIV element | function in TIP |
| Gag | 1 | 634 | 5′ LTR | promoter required to respond to Tat |
| 5′ part of Pol | | | | transactivation element |
| (1074 nt) | 635 | 789 | gag leader | various cis-acting sequences required for |
| Rev | | | sequence | packaging and reverse transcription |
| Vpu | 686 | 823 | psi element | major packaging signal |
| Env | 790 | 2292 | gag orf | polyprotein (structural) |
| cis-acting | 2085 | 3158 | truncated pol ORF | truncated polyprotein (believed to give TIP its |
| elements | | | | genome packaging advantage) |
| 5′ LTR | 4781 | 4903 | cPPT | cis-acting element required for efficient |
| psi | | | | genome transmission |
| packaging | 5738 | 5849 | none | end of vpr sequence with no coding potential |
| signal | 5830 | 6044 | tat exon l | sequence for exon l of tat transactivator |
| gag-leader | | | *mutated* | protein but mutated to prevent translation |
| sequence | 5872 | 5880 | mutations to | Tat knockout makes TIP transcriptionally |
| cPPT | | | make two stop | repressed until infection with HIV |
| RRE | | | codons | |
| PPT | 5969 | 6044 | rev exon 1 | protein that exports viral transcripts (that |
| 3′ LTR | | | | have an RRE) from the nucleus into the |
| MSD | | | | cytoplasm |
| D4 | 6045 | 6060 | none | |
| A3 | 6061 | 6306 | vpu orf | an HIV accessory protein |
| A4 | 6221 | 8785 | env orf | envelope protein |
| A5 | 7759 | 7992 | RRE | rev-response element, cis-acting sequence |
| A7 | | | | required for exporting some viral transcripts |
| | | | | from nucleus |
| | 8369 | 8414 | tat exon 2 | although the tat transcript is made, it is not |
| | | | | translated (see tat exon 1 info) |
| | 8369 | 8643 | rev exon 2 | (see rev exon l for info) |
| | 9049 | 9709 | 3′ LTR | termination of viral genome/required for |
| | | | | reverse transcription and integration |
| | DELETED | | | |
| DELETED/ | coordinates in | | | |
| MUTATED | NL4-3 genome | | | |
| proteins | start | end | HIV element | function in TIP |
| 3′ sequence | 3159 | 4780 | 5′ pol deletion | causes pol truncation |
| of Pol(1938 | | | (part 1 of deletion) | |
| nt) | 4904 | 5737 | 3′ pol-vif-vpr | causes pol truncation, removes splice donors |
| Vif | | | deletion (part 11 | and acceptors: D2, D3, A1 and A2 |
| Vpr | | | of deletion) | |
| Tat (stop | 8786 | 9048 | nef deletion | replaced with GFP |
| codons) | | | | |
| Nef | | | | |
| cis-acting | | | | |
| element | | | | |
| D2 | | | | |
| D3 | | | | |
| A1 | | | | |
| A2 | | | | |

TABLE 2
F1pol splice

| | RETAINED | | | |
|---|---|---|---|---|
| | nt coordinates | | | |
| | in NL4-3 | | | |
| RETAINED | genome | | | |
| proteins | start | end | HIV element | function in TIP |
| gag | 1 | 634 | 5′ LTR | promoter required to respond to Tat |
| 5′ part of Pol | | | | transactivation element |
| (1074 nt) | 635 | 789 | gag leader | various cis-acting sequences required for |
| cis-acting | | | sequence | packaging and reverse transcription |
| elements | 686 | 823 | psi element | major packaging signal |
| 5′ LTR | 790 | 2292 | gag ORF | polyprotein (structural) |
| gag-leader | 2085 | 3158 | truncated pol | truncated polyprotein (believed to give TIP its |
| sequence | | | ORF | genome packaging advantage) |
| psi packaging | 4781 | 4903 | cPPT | cis-acting element required for efficient genome |
| signal | | | | transmission |
| cPPT | 5738 | 5849 | none | partial vpr sequence with no coding potential |
| RRE | 6342 | 8785 | none | partial env sequence with no coding potential |
| PPT | 7759 | 7992 | RRE | rev-response element, cis-acting sequence |
| 3′ LTR | | | | required for exporting some viral transcripts from |
| MSD | | | | nucleus |
| A3 | 8369 | 8414 | none | Tat exon II sequence but because exon I is |
| A7 | | | | deleted, Tat protein is not made |
| | 8369 | 8643 | none | Rev exon 11 sequence but because exon I is |
| | | | | deleted, Rev protein is not made |
| | 9049 | 9709 | 3′ LTR | termination of viral genome/required for reverse |
| | | | | transcription and integration |
| | DELETED | | | |
| DELETED/ | coordinates in | | | |
| MUTATED | NL4-3 genome | | | |
| proteins | start | end | HIV element | function in TIP |
| 3′ part of pol | 3159 | 4780 | 5′ pol deletion (part I | causes pol truncation |
| (1938 nt) | | | of deletion) | |
| Vif | 4904 | 5737 | 3′ pol-vif-vpr deletion | causes pol truncation, removes splice donors and |
| Vpr | | | (part II of deletion) | acceptors: D2, D3, A1 and A2 |
| Tat | 5850 | 6341 | Tat exon I, rev exon I, | knocks out expression of Tat, Rev, Vpu and Env, |
| Rev | | | vpu, beginning of env | removes splice donor and acceptors: D4, A4 and |
| Vpu | | | | A5 |
| Env | 8786 | 9048 | nef deletion | replaced with GFP |
| Nef | | | | |
| cis-acting | | | | |
| elements | | | | |
| D2 | | | | |
| D3 | | | | |
| D4 | | | | |
| A1 | | | | |
| A2 | | | | |
| A4 | | | | |
| A5 | | | | |

TABLE 3
F1vpr

| | RETAINED | | | |
|---|---|---|---|---|
| | nt coordinates | | | |
| | in NL4-3 | | | |
| RETAINED | genome | | | |
| proteins | start | end | HIV element | function in TIP |
| Gag | 1 | 634 | 5′ LTR | promoter required to respond to Tat transactivation |
| 5′ sequence of | | | | element |
| Pol (1074 nt) | 635 | 789 | gag leader | various cis-acting sequences required for packaging |
| 3′ sequence of | | | sequence | and reverse transcription |
| Vpr (219 nt) | 686 | 823 | psi element | major packaging signal |
| Rev | 790 | 2292 | gag ORF | polyprotein (structural) |
| Vpu | 2085 | 3158 | truncated | fusion between Pol polyprotein and Vpr protein |
| Env | 5631 | 5849 | pol/vpr fusion | (believed to give TIP its genome packaging advantage) |
| cis-acting | | | protein | |
| elements | 4778 | 4898 | cPPT* | cis-acting element required for efficient genome |
| 5¹ LTR | | | (inserted after | transmission |
| gag-leader | | | vpr) | |
| sequence | 5850 | 6044 | partial tat exon | beginning of Tat exon moved to other part of genome, |
| psi packaging | | | I (non-coding) | so no coding potential |
| signal | 5969 | 6044 | rev exon 1 | protein that exports viral transcripts (that have the |
| cP PT | | | | RRE) from the nucleus into the cytoplasm |
| RRE | 6045 | 6060 | none | |
| PPT | 6061 | 6306 | vpu orf | an HIV accessory protein |
| 3′ LTR | 6221 | 8785 | env orf | envelope protein |
| MSD | 7759 | 7992 | RRE | rev-response element, cis-acting sequence required for |
| D4 | | | | exporting some viral transcripts from nucleus |
| A3 | 8369 | 8414 | none (tat exon | beginning of tat exon I missing, so no coding potential |
| A4 | | | 2) | (see tat exon 1 info) |
| A5 | 8369 | 8643 | rev exon 2 | (see rev exon 1 for info) |
| A7 | 9049 | 9709 | 3′ LTR | termination of viral genome/required for reverse |
| | | | | transcription and integration |
| | DELETED | | | |
| DELETED/ | coordinates in | | | |
| MUTATED | NL4-3 genome | | | |
| proteins | start | end | HIV element | function in TIP |
| 3′ sequence of | 3159 | 5630 | pol through | this deletion makes the novel truncated pol/vpr fusion |
| Pol (1938 nt) | | | vpr | ORF, removes splice donors and acceptors: D2, D3, A1 |
| Vif | | | deletion/fusion | and A2 |
| 5′ sequence of | 8786 | 9048 | nef deletion | replaced with GFP |
| Vpr (72 bp) | | | | |
| Tat | | | | |
| Nef | | | | |
| cis-acting | | | | |
| element | | | | |
| D2 | | | | |
| D3 | | | | |
| A1 | | | | |
| A2 | | | | |

TABLE 4
F1vpr splice

| | RETAINED | | | |
|---|---|---|---|---|
| | nt coordinates | | | |
| | in NL4-3 | | | |
| RETAINED: | genome | | | |
| proteins | start | end | HIV element | function in TIP |
| gag | 1 | 634 | 5′ LTR | promoter required to respond to Tat transactivation |
| 5′ sequence | | | | element |
| of Pol (1074 | 635 | 789 | gag leader | various cis-acting sequences required for packaging |
| nt) | | | sequence | and reverse transcription |
| 3′ sequence | 686 | 823 | psi element | major packaging signal |
| of Vpr (219 | 790 | 2292 | gag ORF | polyprotein (structural) |
| nt) | 2085 | 3158 | truncated pol/vpr | fusion between Pol polyprotein and Vpr protein |
| cis-acting | 5631 | 5849 | fusion protein | (believed to give TIP its genome packaging |
| elements | | | | advantage) |
| 5′ LTR | 4778 | 4898 | cPPT* (inserted | cis-acting element required for efficient genome |
| psi | | | after vpr) | transmission |
| packaging | 6342 | 8785 | none | partial env sequence with no coding potential |
| signal | 7759 | 7992 | RRE | rev-response element, cis-acting sequence required |
| gag-leader | | | | for exporting some viral transcripts from nucleus |
| sequence | 8369 | 8414 | none | Tat exon II sequence but because exon I is deleted, |
| cPPT | | | | Tat protein is not made |
| PPT | 8369 | 8643 | none | Rev exon II sequence but because exon 1 is deleted, |
| 3′ LTR | | | | Rev protein is not made |
| MSD | 9049 | 9709 | 3′ LTR | termination of viral genome/required for reverse |
| A3 | | | | transcription and integration |
| A7 | | | | |
| | DELETED | | | |
| DELETED/ | coordinates in | | | |
| MUTATED: | NL4-3 genome | | | |
| proteins | start | end | HIV element | function in TIP |
| 3′ sequence | 3159 | 5630 | poi through vpr | this deletion makes the novel truncated pol/vpr |
| of Pol (1938 | | | deletion/fusion | fusion ORF, removes splice donors and acceptors: |
| nt) | | | | D2, D3, A1 and A2 |
| Vif | 5850 | 6341 | Tat exon I, rev exon | knocks out expression of Tat, Rev, Vpu and Env, |
| 5′ sequence | | | I, vpu, beginning of | removes splice donor and acceptors: D4, A4 and A5 |
| of Vpr (72 bp) | | | env | |
| Tat | 8786 | 9048 | nef deletion | replaced with GFP |
| Rev | | | | |
| Vpu | | | | |
| Env | | | | |
| Nef | | | | |
| eis-acting | | | | |
| element | | | | |
| D2 | | | | |
| D3 | | | | |
| D4 | | | | |
| Al | | | | |
| A2 | | | | |
| A4 | | | | |
| A5 | | | | |

### Example 2: Materials and Methods

Cell lines. All cells were grown at 37° C. with 5% CO₂. MT-4 (NIH AIDS Reagent Program, 120) and CEM CD4+(NIH AIDS Reagent Program, 117) cells were maintained in RPMI 1640 (Fisher Scientific, MT10040CV) supplemented with 10% FBS (Fisher Scientific, MT35011CV) and 1% Pen/Strep (Fisher Scientific, MT30002CI). 293 Ts (ATCC, CRL-3216) were maintained in DMEM (Fisher Scientific, MT10013CV) supplemented as for RPMI.

Simulations

Virostat infection and analysis. Once virostats were constructed, cells were added at ˜0.5-1 million cells/mL in 80-120 mL volumes and cultures were monitored for cell density and viability for one to two weeks to ensure stable cell concentration and viability. Once cultures were stable, cells were infected with replication competent HIV (expressing either GFP or BFP) at low MOI (˜0.01-0.1, depending on experiment). On indicated days post infection, 3 mL of total flask contents (i.e. cells and supernatant) were removed from flasks and analyzed. Cell concentration and viability were determined using a Bio-Rad TC20 Automated cell counter (Catalog #1450102), cells were fixed in 1% formaldehyde (Tousimis Research Corporation, 1008A) and fluorescence was measured on a BD LSR II flow cytometer and analyzed in FlowJo. Virostat supernatants were harvested by pelleting cells at 1,000 g for 3 minutes, removing supernatants to clean tubes and freezing at −80. Supernatants were filtered upon thawing for downstream analysis. For the original virostat, viral titers were determined by TCID₅₀ as indicated below. For virostats started with TIP cells, the frequency of BFP+ cells was used as a proxy for viral load since the full-length HIV construct harbored a BFP tag.

PCR and Sanger sequencing of proviral DNA. Total genomic DNA was extracted from ˜1 million virostat cells using the NucleoSpin Tissue kit (740052.50) according to the manufacturer's protocol. PCR was performed with primers Pol forward and Vpu reverse (see Primers table) using the Phusion High-Fidelity PCR Master Mix (NEB, M0531S) and the following conditions: initial denaturation: 98° C. for 30 seconds; cycle 30 times: 98° C. for 10 seconds, 60° C. for 30 seconds, 72° C. for 4 minutes; final extension: 72° C. for 10 min, hold at 4° C. PCR products were analyzed on a 0.8% agarose gel and excised for sequencing. PCR products were gel extracted using the QIAquick gel extraction kit (Qiagen, 28115) and sent to Elim Biopharm for sequencing confirmation using the Pol forward and Vpu reverse primers (see Primers table).

Plasmids: The mutations for all F1 constructs and other deletion variants are listed in Tables 1-4. Cloning was done by various PCR cloning techniques as convenient. Restriction cloning was used to exchange plasmid sequences as necessary. Sequences were confirmed by Sanger sequencing at Elim Biopharm.

Transfection for viral stocks, lentivirus or co-transfection competition experiments. For full-length HIV stocks, 293T cells were transfected with plasmid pNLENG1-IRES-Nef (Levy et al., 2005) or derivatives thereof (see Tables 1-4). To package TIP constructs or lentiviral vectors, the transfer plasmid, packaging plasmid pCMV-dR8.91 (Zufferey et al., 1997) and pseudotyping plasmid pCMV-VSVG (Addgene, #8454) were mixed at a mass ratio of 4:10:5, respectively, except for TIP constructs prepared for primary cell transduction which were mixed at a 1:2:1 ratio. Co-transfection competitions were performed by mixing plasmids at the indicated molar ratios and equalizing the mass across all reactions with excess pUC19. To make the transfection mix, plasmids were diluted in unsupplemented DMEM (i.e. no serum or antibiotics added) to a concentration of 10 ng/mL total DNA and PEI was added to a concentration of 30 μg/mL in a volume˜10% of the total volume in the transfection well or dish (e.g. 200 μl for a 6-well plate with 2 mL media). The transfection mix was vortexed vigorously, incubated for 10 minutes at room temperature, added to cells and incubated overnight. Transfection supernatant was replaced with fresh, complete DMEM the following day and supernatant was collected around 48 hours post transfection. Cell debris was removed by centrifugation and supernatants were filtered through a 0.45 μm filter. Concentrated virus was prepared by ultracentrifugation (Beckman Coulter Optima XE-90, rotor SW 32 Ti) at 20,000 rpm through a 6% iodixanol gradient (Sigma Aldrich, D1556-250 mL) for 1.5-2 hours.

Viral and lentiviral titers. Fluorescently-tagged full-length HIV and lentivirus were titered by serial dilution on MT-4s and analyzed by flow cytometry on an BD LSR II or MACSQuant VYB flow cytometer at 2 dpi. Lentiviral stocks with weak expression from the LTR (i.e. constructs without Tat) were titered by transducing MT-4s, superinfecting with full-length HIV at 2 or 3 days post transduction (to provide Tat in trans) and then measured as the percent of infected cells that were positive for lentiviral expression at 2 dpi. All fluorescence data was analyzed in FlowJo. Original virostat titers were determined by TCID₅₀ in case the GFP tag was lost through mutation over the course of the long-term infection

Supernatant p24 concentration. The supernatant concentration of p24 was determined by the FLAQ method (Hayden et al., 2003 and Gesner et al., 2014), essentially a sandwich ELISA performed on beads using a PE-linked antibody to enable quantification by flow cytometry. Briefly, serially diluted supernatant samples were incubated with KC57-PE p24 antibody (Beckman Coulter, 6604667) and polystyrene beads previously cross-linked to Human anti-p24 Gag HIV-1 IIIB polyclonal antibodies (ImmunoDC LLC, 2503). After washing and fixing, bead fluorescence was analyzed by flow cytometry on a BD LSR II and fluorescence intensity was converted to concentration using a standard curve in FlowJo.

Transduction efficiency calculation. The efficiency of lentiviral stock transduction was measured as the transduction units (“titer”) per ng of p24 (i.e. capsid protein). Titers and p24 concentration were determined as described above.

Construction of single-integration polyclonal DIP/TIP cell lines. Polyclonal cell lines were created by transducing CEMs with lentivirus at low MOI (to ensure single integrations) and batch sorting GFP or BFP-expressing cells (depending on construct used) using a BD FACSAria II cell sorter. Prior to sorting, transduced populations were treated with 5 μM Darunavir for 3-5 days to eliminate replication-competent recombinants. NB: Although GFP or BFP expression was very low for most constructs due to the absence of Tat, dim expression was detectable on the BD FACSAria II.

Cell line infection, analysis and supernatant harvest. Cell lines (CEM or MT-4) transduced with TIP constructs (or controls) were superinfected with fluorescently-tagged HIV at a density of 0.5 million cells/mL. The frequency of infection was monitored by fixing cells in 1% formaldehyde (Tousimis Research Corporation, 1008A), measuring fluorescent cells by flow cytometry on a BD LSR II and analyzing in FlowJo. For single cycle infection analyses, supernatants were collected at 3 dpi by pelleting cells, filtering supernatant through a 0.45 μM filter (Millex, SLHV033RS).

CD4 receptor staining and analysis. Cells were stained with a Brilliant Violet 650-conjugated anti-human CD4 antibody (Biolegend, 317436) or isotype control (Biolegend, 400351) using 1 μl of antibody for 0.5 million cells in a 0.1 mL volume. Cells were incubated with antibody for 30 minutes protected from light, washed with 1 mL flow buffer (2% FBS, 2 mM EDTA in DPBS) and resuspended in 0.25 mL fixing solution (1% formaldehyde in flow buffer). CD4 staining was assessed by flow cytometry on a BD LSR II and analyzed in FlowJo.

Analysis of viral RNA by RT-qPCR. Viral genomic RNA was extracted from filtered supernatants using the QIAamp Viral RNA Mini Kit (Qiagen, 52904) according to the manufacturer's protocol, 2 μl of a 1:1,000 dilution of MS2 RNA solution (Sigma-Aldrich, 10165948001) was also spiked in as a normalization control. Extracted viral RNA was DNase treated with the TURBO DNA-free kit (Thermo Fisher Scientific, AM1907) according to routine DNase treatment protocol. Total cellular RNA was extracted using the Qiagen RNeasy Mini Kit (Qiagen, 74104) according to the manufacturer's protocols including the on-column DNA digestion (RNase-free DNase I, Qiagen, 79254). RNA was reverse transcribed using either the QuantiTect Reverse Transcription Kit (Qiagen, 205311) according to the manufacturer's protocols (including the gDNA wipeout step) or M-MuLV reverse transcriptase (NEB, M0253L) and Murine RNase Inhibitor (NEB, M0314S) according to manufacturer's standard protocol including the recommended 25° C. incubation for random primers, except that samples were denatured at 70° C. and the enzyme was inactivated at 80° C. for 5 min. For all cDNA synthesis, a no-RT enzyme control was included and reactions were primed with random hexamers. qPCR was performed using the Fast SYBR Green master mix (ThermoFisher Scientific, 4385612) according to manufacturer's protocols using a 10 μl reaction volume and 200 nM final primer concentration on an Applied Biosystems 7500 fast real-time PCR system. Primers for total, full-length and TIP gRNA as well as MS2 control RNA can be found in the Primers table. The percent of full-length RNA was determined by converting total and full-length Ct values to fold change (normalized to full-length only samples) and dividing the WT fold change by the total fold change. TIP RNA was assumed to be the remaining gRNA in the sample.

HIV-1-humanized mouse challenge (adapted from Leslie et al. PLoS Pathog, 2016). NOD.Cg-Prkdc^{scid} Il2rg^{tm1Wjl}/SzJ (NSG) mice were engrafted with 2×10⁷ unmodified primary human CD4 T cells or a 50/50 mix of unmodified and TIP-transduced primary human CD4 T cells (i.e. ˜1×10⁷ untransduced cells and ˜1×10⁷TIP cells), and monitored for engraftment of CD45⁺CD4⁺ cells after 3 weeks by TruCount (Becton Dickinson) analysis using 50 μL of blood from each animal. Mice were then normalized based on engraftment into groups of 5 control (uninfected) and 8 test (infected) animals per experimental group. Mice were infected with IU BaL full-length HIV bled every 7 days for 3 weeks and CD4+ T cell counts were analyzed by TruCount. TruCount analysis was performed using PerCP-Cy5.5 conjugated anti-human CD45 antibody (eBioscience), and BV421-conjugated anti-CD4 antibody (eBioscience). Approximately 50 μl serum was saved for viral load analysis when available. Animals were housed at the University of Pennsylvania.

Mouse serum RNA extraction. RNA was extracted from mouse serum using the BD TRI Reagent (Molecular Research Center, TB 126) according to manufacturer's protocol for RNA extraction from serum. Before beginning the protocol, 150 μl DPBS was added to the ˜50 μl serum sample to bring the volume to ˜200 μl. Final resuspension of RNA was performed in 50 μl of nuclease-free water.

ddPCR and analysis of mouse RNA samples. Digital Droplet PCR was performed using the Bio-Rad QX100 droplet generator (1863002) and reader (1863003) according to the manufacturer's protocol. RT-PCR reactions were carried out using the Bio-Rad One-Step RT-ddPCR Advanced Kit for Probes (BioRad, 1864021) following the manufacturer's protocol with the following reaction conditions: 900 nM primers, 250 nM Taqman target probes and a 1× concentration of serum-extracted RNA. For thermocycling conditions, initial denaturation was 65° C. for 5 min, and annealing and extension was performed at 56° C. for 1 min, 5 sec. Data were acquired and analyzed with QuantaSoft Software. See primers table for list of ddPCR primers and probes. All reactions were performed in technical duplicate and samples below the limit of detection (determined by negative controls) were not analyzed for TIP content.

Statistical analysis. For titer or qPCR comparisons, p-values were obtained using an unpaired T test. For TIP virostat infection trajectories over time, a paired t-test was used. For CD4+ T cell concentration in mouse experiments, intra- and inter-group comparisons were done using a Mann-Whitney test and viral loads were analyzed by one-way ANOVA and the Tukey's test for multiple comparisons.

### BIOGRAPHY

Gesner M, Maiti M, Grant R and Cavrois M. Fluorescence-linked antigen quantification (FLAQ) assay for fast quantification of HIV-1 p24 Gag. Bio-protocol, 2014. 4(24): e1366.
Hayden M S, Palacios E H and Grant R M. Real-time quantitation of HIV-1 p24 and SIV p27 using fluorescence-linked antigen quantification assays. AIDS, 2003. 17 (4): 629-631.
Leslie G J, Wang J, Richardson M W et al. Potent and broad inhibition of HIV-1 by a peptide from the gp41 heptad repeat-2 domain conjugated to the CXCR4 amino terminus. PLoS Pathogens, 2016. 12(11): e1005983.
Levy D N, Aldrovandi G M, Kutsch O and Shaw G M. Dynamics of HIV-1 recombination in its natural target cells. PNAS, 2004. 101 (12): 4204-9.
Zufferey R, Nagy D, Mandel R J, Naldini L and Trono D. Multiply attenuated lentiviral vector achieves efficient gene delivery in vivo. Nature Biotechnology, 1997. 15(9): 871-5.
U.S. Pat. No. 7,572,906.
Metzger et al. (2011) PLoS Comp. Biol. 7:e1002015.
Dull et al. (1998) J. Virol. 72:8463-71.
Huang and Baltimore (1970) Nature 226:325-7.
Levine et al. (2006) Proc. Natl. Acad Sci. USA 103:17372-7.
Weinberger et al. (2003) J. Virol. 77:10028-36.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An interfering, conditionally replicating and conditionally mobilizing, recombinant human immunodeficiency virus (HIV) construct, the construct comprising:
a) cis-acting elements comprising 5′ and 3′ long terminal repeat, a Gag-leader sequence, a Ψ packaging signal, a central polypurine tract (cPPT), a rev response element (RRE) sequence, a polypurine tract (PPT), a major splice donor (MSD), A3, and A7; and
b) one or more alterations in an HIV nucleotide sequence, wherein the one or more alterations renders each of Pol, Tat, Vpr, Nef, and Vif nonfunctional such that the construct is incapable of replication and production of virus on its own but requires replication-competent HIV to act as a helper virus,
wherein the construct further comprises one or more alterations that renders each of Rev, Vpu, and Env nonfunctional.

2. The construct of claim 1, wherein the cis-acting elements further comprise D4, A4, and A5.

3. (canceled)

4. (canceled)

5. The construct of claim 1, wherein genomic RNA (gRNA) encoded by the construct is produced at a higher rate than wild-type HIV gRNA when present in a host cell infected with a wild-type HIV, such that the ratio of the gRNA encoded by the construct to the wild-type HIV gRNA is higher than about 1 in the cell.

6. The construct of claim 5, wherein the construct has a higher cell-to-cell transmission frequency than the wild-type HIV.

7. The construct of claim 1, wherein the construct has a basic reproductive ratio (R0)>1 in the presence of HIV-1.

8. The construct of claim 1, wherein the construct does not include any heterologous nucleotide sequences that encode a gene product.

9. The construct of claim 1, wherein the construct is packaged with an equal or higher efficiency than wild-type HIV when present in a host cell infected with a wild-type HIV.

10. The construct of claim 1, wherein the cis-acting elements include at least one cis element embedded within an HIV protein-coding sequence.

11. The construct of claim 1, further comprising i) one or more alterations comprising a deletion or mutation in an HIV splice donor site, wherein D2 and D3 splice donor sites are deleted, ii) further comprising one or more alterations comprising a deletion or mutation in an HIV splice acceptor site, wherein A1 and A2 splice acceptor sites are deleted: or iii) a combination of i) and ii).

12. (canceled)

13. (canceled)

14. (canceled)

15. (canceled)

16. The construct of claim 1, wherein the construct comprises a deletion of the nucleotides corresponding to positions 3159 to 4780, 4904 to 5737, and 8786 to 9048 numbered relative to the reference HIV sequence of SEQ ID NO: 1.

17. The construct of claim 16, wherein the construct comprises the nucleotides corresponding to positions 1 to 634, 635 to 789, 686 to 823, 790 to 2292, 2085 to 3158, 4781 to 4903, 5738 to 5849, 5830 to 6044, 5872 to 5880, 5969 to 6044, 6045 to 6060, 6061 to 6306, 6221 to 8785, 7759 to 7992, 8369 to 8414, 8369 to 8643, and 9049 to 9709 numbered relative to the reference HIV sequence of SEQ ID NO:1.

18. The construct of claim 16, wherein the construct comprises or consists of the nucleotide sequence of SEQ ID NO:2, or a nucleotide sequence having at least 90% identity to the nucleotide sequence of SEQ ID NO:2.

19. The construct of claim 16, further comprising a deletion or a mutation of one or more nucleotides in the nucleotide sequence encoding HIV Rev, a deletion or a mutation of one or more nucleotides in the nucleotide sequence encoding HIV Vpu, and a deletion or a mutation of one or more nucleotides in the nucleotide sequence encoding HIV Env.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. The construct of claim 1, wherein the construct comprises a deletion in the nucleotide sequence encoding HIV Gag rendering a subset or all gag proteins non-functional.

38. A pharmaceutical composition comprising the construct of claim 1 and a pharmaceutically acceptable excipient.

39. (canceled)

40. A method of reducing human immunodeficiency virus viral load in an individual, the method comprising administering to the individual an effective amount of the pharmaceutical composition of claim 38.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. An isolated biological fluid comprising the construct of claim 1.

53. (canceled)

54. A method of generating a variant interfering, conditionally replicating, human immunodeficiency virus (HIV) construct, the method comprising:
a) introducing the construct of claim 1 into a first individual;
b) obtaining a biological sample from a second individual to whom the construct of claim 1 has been transmitted from the first individual, wherein the construct present in the second individual is a variant of the construct of claim 1; and
c) cloning the variant construct from the second individual.

55. (canceled)

56. (canceled)

57. (canceled)


SEQUENCE LISTING (20 sequences)

SEQ ID NO: 1 - DNA, 9000 bp, Human immunodeficiency virus
  ref <308>: DATABASE ACCESSION NUMBER: NCBI/U26942
  ref <309>: DATABASE ENTRY DATE: 1995-11-15
  ref <313>: RELEVANT RESIDUES IN SEQ ID NO: (1)..(9000)
aaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcactgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaggcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacgtagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttagaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtggcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcgggaatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagtcctaggtgtgaatatcaagcaggacataacaaggtaggatctctacagtacttggcactagcagcattaataaaaccaaaacagataaagccacctttgcctagtgttaggaaactgacagaggacagatggaacaagccccagaagaccaagggccacagagggagccatacaacgaatggacactagagcttttagaggaacttaagagtgaagctgttagacattttcctaggatatggctccataacttaggacgacatatctatgaaacttacggggatacttgggcaggagtggaagccataataagaattctgcaacaactgccgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaagatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaatccccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagaggcagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagggc

SEQ ID NO: 2 - DNA, 12888 bp, Artificial Sequence
  F1pol plasmid
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagagttttaaaagaaaaggggggattggggggtacagtgcaggggaaagaatagtagacataatagcaacagacatacaaactaaagaattacaaaaacaaattacaaaaattcaaaattttcgggtaataagaattctgcaacaactgctgtttatccatttcagaattgggtgtcgacatagcagaataggcgttactcgacagaggagagcaagaaatggagccagtagatcctagactagagccctggaagcatccatgaagttagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcaccccccaggaggtagaggttgcagtgagccaagatcgcgccactgcattccagcctgggcaagaaaacaagactgtctaaaataataataataagttaagggtattaaatatatttatacatggaggtcataaaaatatatatatttgggctgggcgcagtggctcacacctgcgcccggccctttgggaggccgaggcaggtggatcacctgagtttgggagttccagaccagcctgaccaacatggagaaaccccttctctgtgtatttttagtagattttattttatgtgtattttattcacaggtatttctggaaaactgaaactgtttttcctctactctgataccacaagaatcatcagcacagaggaagacttctgtgatcaaatgtggtgggagagggaggttttcaccagcacatgagcagtcagttctgccgcagactcggcgggtgtccttcggttcagttccaacaccgcctgcctggagagaggtcagaccacagggtgagggctcagtccccaagacataaacacccaagacataaacacccaacaggtccaccccgcctgctgcccaggcagagccgattcaccaagacgggaattaggatagagaaagagtaagtcacacagagccggctgtgcgggagaacggagttctattatgactcaaatcagtctccccaagcattcggggatcagagtttttaaggataacttagtgtgtagggggccagtgagttggagatgaaagcgtagggagtcgaaggtgtccttttgcgccgagtcagttcctgggtgggggccacaagatcggatgagccagtttatcaatccgggggtgccagctgatccatggagtgcagggtctgcaaaatatctcaagcactgattgatcttaggttttacaatagtgatgttaccccaggaacaatttggggaaggtcagaatcttgtagcctgtagctgcatgactcctaaaccataatttcttttttgtttttttttttttatttttgagacagggtctcactctgtcacctaggctggagtgcagtggtgcaatcacagctcactgcagcctcaacgtcgtaagctcaagcgatcctcccacctcagcctgcctggtagctgagactacaagcgacgccccagttaatttttgtatttttggtagaggcagcgttttgccgtgtggccctggctggtctcgaactcctgggctcaagtgatccagcctcagcctcccaaagtgctgggacaaccggggccagtcactgcacctggccctaaaccataatttctaatcttttggctaatttgttagtcctacaaaggcagtctagtccccaggcaaaaagggggtttgtttcgggaaagggctgttactgtctttgtttcaaactataaactaagttcctcctaaacttagttcggcctacacccaggaatgaacaaggagagcttggaggttagaagcacgatggaattggttaggtcagatctctttcactgtctgagttataattttgcaatggtggttcaaagactgcccgcttctgacaccagtcgctgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaactgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtactggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccaggggaggcagagattgcagtaagctgagatcgcagcactgcactccagcctgggcgacagagtaagactctgtctcaaaaataaaataaataaatcaatcagatattccaatcttttcctttatttatttatttattttctattttggaaacacagtccttccttattccagaattacacatatattctatttttctttatatgctccagttttttttagaccttcacctgaaatgtgtgtatacaaaatctaggccagtccagcagagcctaaaggtaaaaaataaaataataaaaaataaataaaatctagctcactccttcacatcaaaatggagatacagctgttagcattaaataccaaataacccatcttgtcctcaataattttaagcgcctctctccaccacatctaactcctgtcaaaggcatgtgccccttccgggcgctctgctgtgctgccaaccaactggcatgtggactctgcagggtccctaactgccaagccccacagtgtgccctgaggctgccccttccttctagcggctgcccccactcggctttgctttccctagtttcagttacttgcgttcagccaaggtctgaaactaggtgcgcacagagcggtaagactgcgagagaaagagaccagctttacagggggtttatcacagtgcaccctgacagtcgtcagcctcacagggggtttatcacattgcaccctgacagtcgtcagcctcacagggggtttatcacagtgcacccttacaatcattccatttgattcacaatttttttagtctctactgtgcctaacttgtaagttaaatttgatcagaggtgtgttcccagaggggaaaacagtatatacagggttcagtactatcgcatttcaggcctccacctgggtcttggaatgtgtcccccgaggggtgatgactacctcagttggatctccacaggtcacagtgacacaagataaccaagacacctcccaaggctaccacaatgggccgccctccacgtgcacatggccggaggaactgccatgtcggaggtgcaagcacacctgcgcatcagagtccttggtgtggagggagggaccagcgcagcttccagccatccacctgatgaacagaacctagggaaagccccagttctacttacaccaggaaaggc

SEQ ID NO: 3 - DNA, 12396 bp, Artificial Sequence
  F1pol splice plasmid
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagagttttaaaagaaaaggggggattggggggtacagtgcaggggaaagaatagtagacataatagcaacagacatacaaactaaagaattacaaaaacaaattacaaaaattcaaaattttcgggtaataagaattctgcaacaactgctgtttatccatttcagaattgggtgtcgacatagcagaataggcgttactcgacagaggagagcaagaaatggagccagtagatcctaggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcaccccccaggaggtagaggttgcagtgagccaagatcgcgccactgcattccagcctgggcaagaaaacaagactgtctaaaataataataataagttaagggtattaaatatatttatacatggaggtcataaaaatatatatatttgggctgggcgcagtggctcacacctgcgcccggccctttgggaggccgaggcaggtggatcacctgagtttgggagttccagaccagcctgaccaacatggagaaaccccttctctgtgtatttttagtagattttattttatgtgtattttattcacaggtatttctggaaaactgaaactgtttttcctctactctgataccacaagaatcatcagcacagaggaagacttctgtgatcaaatgtggtgggagagggaggttttcaccagcacatgagcagtcagttctgccgcagactcggcgggtgtccttcggttcagttccaacaccgcctgcctggagagaggtcagaccacagggtgagggctcagtccccaagacataaacacccaagacataaacacccaacaggtccaccccgcctgctgcccaggcagagccgattcaccaagacgggaattaggatagagaaagagtaagtcacacagagccggctgtgcgggagaacggagttctattatgactcaaatcagtctccccaagcattcggggatcagagtttttaaggataacttagtgtgtagggggccagtgagttggagatgaaagcgtagggagtcgaaggtgtccttttgcgccgagtcagttcctgggtgggggccacaagatcggatgagccagtttatcaatccgggggtgccagctgatccatggagtgcagggtctgcaaaatatctcaagcactgattgatcttaggttttacaatagtgatgttaccccaggaacaatttggggaaggtcagaatcttgtagcctgtagctgcatgactcctaaaccataatttcttttttgtttttttttttttatttttgagacagggtctcactctgtcacctaggctggagtgcagtggtgcaatcacagctcactgcagcctcaacgtcgtaagctcaagcgatcctcccacctcagcctgcctggtagctgagactacaagcgacgccccagttaatttttgtatttttggtagaggcagcgttttgccgtgtggccctggctggtctcgaactcctgggctcaagtgatccagcctcagcctcccaaagtgctgggacaaccggggccagtcactgcacctggccctaaaccataatttctaatcttttggctaatttgttagtcctacaaaggcagtctagtccccaggcaaaaagggggtttgtttcgggaaagggctgttactgtctttgtttcaaactataaactaagttcctcctaaacttagttcggcctacacccaggaatgaacaaggagagcttggaggttagaagcacgatggaattggttaggtcagatctctttcactgtctgagttataattttgcaatggtggttcaaagactgcccgcttctgacaccagtcgctgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaactgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtactggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccaggggaggcagagattgcagtaagctgagatcgcagcactgcactccagcctgggcgacagagtaagactctgtctcaaaaataaaataaataaatcaatcagatattccaatcttttcctttatttatttatttattttctattttggaaacacagtccttccttattccagaattacacatatattctatttttctttatatgctccagttttttttagaccttcacctgaaatgtgtgtatacaaaatctaggccagtccagcagagcctaaaggtaaaaaataaaataataaaaaataaataaaatctagctcactccttcacatcaaaatggagatacagctgttagcattaaataccaaataacccatcttgtcctcaataattttaagcgcctctctccaccacatctaactcctgtcaaaggcatgtgccccttccgggcgctctgctgtgctgccaaccaactggcatgtggactctgcagggtccctaactgccaagccccacagtgtgccctgaggctgccccttccttctagcggctgcccccactcggctttgctttccctagtttcagttacttgcgttcagccaaggtctgaaactaggtgcgcacagagcggtaagactgcgagagaaagagaccagctttacagggggtttatcacagtgcaccctgacagtcgtcagcctcacagggggtttatcacattgcaccctgacagtcgtcagcctcacagggggtttatcacagtgcacccttacaatcattccatttgattcacaatttttttagtctctactgtgcctaacttgtaagttaaatttgatcagaggtgtgttcccagaggggaaaacagtatatacagggttcagtactatcgcatttcaggcctccacctgggtcttggaatgtgtcccccgaggggtgatgactacctcagttggatctccacaggtcacagtgacacaagataaccaagacacctcccaaggctaccacaatgggccgccctccacgtgcacatggccggaggaactgccatgtcggaggtgcaagcacacctgcgcatcagagtccttggtgtggagggagggaccagcgcagcttccagccatccacctgatgaacagaacctagggaaagccccagttctacttacaccaggaaaggc

SEQ ID NO: 4 - DNA, 12996 bp, Artificial Sequence
  F1vpr plasmid
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctagcaattttaaaagaaaaggggggattggggggtacagtgcaggggaaagaatagtagacataatagcaacagacatacaaactaaagaattacaaaaacaaattacaaaaattcaaaattttgccactagagccctggaagcatccatgaagttagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcaccccccaggaggtagaggttgcagtgagccaagatcgcgccactgcattccagcctgggcaagaaaacaagactgtctaaaataataataataagttaagggtattaaatatatttatacatggaggtcataaaaatatatatatttgggctgggcgcagtggctcacacctgcgcccggccctttgggaggccgaggcaggtggatcacctgagtttgggagttccagaccagcctgaccaacatggagaaaccccttctctgtgtatttttagtagattttattttatgtgtattttattcacaggtatttctggaaaactgaaactgtttttcctctactctgataccacaagaatcatcagcacagaggaagacttctgtgatcaaatgtggtgggagagggaggttttcaccagcacatgagcagtcagttctgccgcagactcggcgggtgtccttcggttcagttccaacaccgcctgcctggagagaggtcagaccacagggtgagggctcagtccccaagacataaacacccaagacataaacacccaacaggtccaccccgcctgctgcccaggcagagccgattcaccaagacgggaattaggatagagaaagagtaagtcacacagagccggctgtgcgggagaacggagttctattatgactcaaatcagtctccccaagcattcggggatcagagtttttaaggataacttagtgtgtagggggccagtgagttggagatgaaagcgtagggagtcgaaggtgtccttttgcgccgagtcagttcctgggtgggggccacaagatcggatgagccagtttatcaatccgggggtgccagctgatccatggagtgcagggtctgcaaaatatctcaagcactgattgatcttaggttttacaatagtgatgttaccccaggaacaatttggggaaggtcagaatcttgtagcctgtagctgcatgactcctaaaccataatttcttttttgtttttttttttttatttttgagacagggtctcactctgtcacctaggctggagtgcagtggtgcaatcacagctcactgcagcctcaacgtcgtaagctcaagcgatcctcccacctcagcctgcctggtagctgagactacaagcgacgccccagttaatttttgtatttttggtagaggcagcgttttgccgtgtggccctggctggtctcgaactcctgggctcaagtgatccagcctcagcctcccaaagtgctgggacaaccggggccagtcactgcacctggccctaaaccataatttctaatcttttggctaatttgttagtcctacaaaggcagtctagtccccaggcaaaaagggggtttgtttcgggaaagggctgttactgtctttgtttcaaactataaactaagttcctcctaaacttagttcggcctacacccaggaatgaacaaggagagcttggaggttagaagcacgatggaattggttaggtcagatctctttcactgtctgagttataattttgcaatggtggttcaaagactgcccgcttctgacaccagtcgctgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaactgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtactggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccaggggaggcagagattgcagtaagctgagatcgcagcactgcactccagcctgggcgacagagtaagactctgtctcaaaaataaaataaataaatcaatcagatattccaatcttttcctttatttatttatttattttctattttggaaacacagtccttccttattccagaattacacatatattctatttttctttatatgctccagttttttttagaccttcacctgaaatgtgtgtatacaaaatctaggccagtccagcagagcctaaaggtaaaaaataaaataataaaaaataaataaaatctagctcactccttcacatcaaaatggagatacagctgttagcattaaataccaaataacccatcttgtcctcaataattttaagcgcctctctccaccacatctaactcctgtcaaaggcatgtgccccttccgggcgctctgctgtgctgccaaccaactggcatgtggactctgcagggtccctaactgccaagccccacagtgtgccctgaggctgccccttccttctagcggctgcccccactcggctttgctttccctagtttcagttacttgcgttcagccaaggtctgaaactaggtgcgcacagagcggtaagactgcgagagaaagagaccagctttacagggggtttatcacagtgcaccctgacagtcgtcagcctcacagggggtttatcacattgcaccctgacagtcgtcagcctcacagggggtttatcacagtgcacccttacaatcattccatttgattcacaatttttttagtctctactgtgcctaacttgtaagttaaatttgatcagaggtgtgttcccagaggggaaaacagtatatacagggttcagtactatcgcatttcaggcctccacctgggtcttggaatgtgtcccccgaggggtgatgactacctcagttggatctccacaggtcacagtgacacaagataaccaagacacctcccaaggctaccacaatgggccgccctccacgtgcacatggccggaggaactgccatgtcggaggtgcaagcacacctgcgcatcagagtccttggtgtggagggagggaccagcgcagcttccagccatccacctgatgaacagaacctagggaaagccccagttctacttacaccaggaaaggc

SEQ ID NO: 5 - DNA, 12510 bp, Artificial Sequence
  F1vpr splice plasmid
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctagcaattttaaaagaaaaggggggattggggggtacagtgcaggggaaagaatagtagacataatagcaacagacatacaaactaaagaattacaaaaacaaattacaaaaattcaaaattttcgggttggagggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcaccccccaggaggtagaggttgcagtgagccaagatcgcgccactgcattccagcctgggcaagaaaacaagactgtctaaaataataataataagttaagggtattaaatatatttatacatggaggtcataaaaatatatatatttgggctgggcgcagtggctcacacctgcgcccggccctttgggaggccgaggcaggtggatcacctgagtttgggagttccagaccagcctgaccaacatggagaaaccccttctctgtgtatttttagtagattttattttatgtgtattttattcacaggtatttctggaaaactgaaactgtttttcctctactctgataccacaagaatcatcagcacagaggaagacttctgtgatcaaatgtggtgggagagggaggttttcaccagcacatgagcagtcagttctgccgcagactcggcgggtgtccttcggttcagttccaacaccgcctgcctggagagaggtcagaccacagggtgagggctcagtccccaagacataaacacccaagacataaacacccaacaggtccaccccgcctgctgcccaggcagagccgattcaccaagacgggaattaggatagagaaagagtaagtcacacagagccggctgtgcgggagaacggagttctattatgactcaaatcagtctccccaagcattcggggatcagagtttttaaggataacttagtgtgtagggggccagtgagttggagatgaaagcgtagggagtcgaaggtgtccttttgcgccgagtcagttcctgggtgggggccacaagatcggatgagccagtttatcaatccgggggtgccagctgatccatggagtgcagggtctgcaaaatatctcaagcactgattgatcttaggttttacaatagtgatgttaccccaggaacaatttggggaaggtcagaatcttgtagcctgtagctgcatgactcctaaaccataatttcttttttgtttttttttttttatttttgagacagggtctcactctgtcacctaggctggagtgcagtggtgcaatcacagctcactgcagcctcaacgtcgtaagctcaagcgatcctcccacctcagcctgcctggtagctgagactacaagcgacgccccagttaatttttgtatttttggtagaggcagcgttttgccgtgtggccctggctggtctcgaactcctgggctcaagtgatccagcctcagcctcccaaagtgctgggacaaccggggccagtcactgcacctggccctaaaccataatttctaatcttttggctaatttgttagtcctacaaaggcagtctagtccccaggcaaaaagggggtttgtttcgggaaagggctgttactgtctttgtttcaaactataaactaagttcctcctaaacttagttcggcctacacccaggaatgaacaaggagagcttggaggttagaagcacgatggaattggttaggtcagatctctttcactgtctgagttataattttgcaatggtggttcaaagactgcccgcttctgacaccagtcgctgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaactgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtactggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccaggggaggcagagattgcagtaagctgagatcgcagcactgcactccagcctgggcgacagagtaagactctgtctcaaaaataaaataaataaatcaatcagatattccaatcttttcctttatttatttatttattttctattttggaaacacagtccttccttattccagaattacacatatattctatttttctttatatgctccagttttttttagaccttcacctgaaatgtgtgtatacaaaatctaggccagtccagcagagcctaaaggtaaaaaataaaataataaaaaataaataaaatctagctcactccttcacatcaaaatggagatacagctgttagcattaaataccaaataacccatcttgtcctcaataattttaagcgcctctctccaccacatctaactcctgtcaaaggcatgtgccccttccgggcgctctgctgtgctgccaaccaactggcatgtggactctgcagggtccctaactgccaagccccacagtgtgccctgaggctgccccttccttctagcggctgcccccactcggctttgctttccctagtttcagttacttgcgttcagccaaggtctgaaactaggtgcgcacagagcggtaagactgcgagagaaagagaccagctttacagggggtttatcacagtgcaccctgacagtcgtcagcctcacagggggtttatcacattgcaccctgacagtcgtcagcctcacagggggtttatcacagtgcacccttacaatcattccatttgattcacaatttttttagtctctactgtgcctaacttgtaagttaaatttgatcagaggtgtgttcccagaggggaaaacagtatatacagggttcagtactatcgcatttcaggcctccacctgggtcttggaatgtgtcccccgaggggtgatgactacctcagttggatctccacaggtcacagtgacacaagataaccaagacacctcccaaggctaccacaatgggccgccctccacgtgcacatggccggaggaactgccatgtcggaggtgcaagcacacctgcgcatcagagtccttggtgtggagggagggaccagcgcagcttccagccatccacctgatgaacagaacctagggaaagccccagttctacttacaccaggaaaggc

SEQ ID NO: 6 - RNA, 40 nt, Artificial Sequence
  HIV-1 SL-1
ggacucggcuugcugaagygcrcwcrgcaagaggcgagrg

SEQ ID NO: 7 - RNA, 19 nt, Artificial Sequence
  HIV-1 SL2
rgcgacuggugaguacgch

SEQ ID NO: 8 - RNA, 23 nt, Artificial Sequence
  HIV-1 SL3
ugacuagcggaggcuagaaggag

SEQ ID NO: 9 - RNA, 20 nt, Artificial Sequence
  HIV-1 SL4
ugggugcgagagcgucarua

SEQ ID NO: 10 - DNA, 19 bp, Human immunodeficiency virus
ggcgactggtgagtacgcc

SEQ ID NO: 11 - DNA, 19 bp, Human immunodeficiency virus
ggcggctggtgagtacgcc

SEQ ID NO: 12 - DNA, 18 bp, Human immunodeficiency virus
ggcgaatggtgagtacgc

SEQ ID NO: 13 - DNA, 19 bp, Human immunodeficiency virus
agcgactggtgagtacgct

SEQ ID NO: 14 - DNA, 19 bp, Human immunodeficiency virus
agcgaccggtgagtacgct

SEQ ID NO: 15 - RNA, 11 nt, Human immunodeficiency virus
aaggugaaggg

SEQ ID NO: 16 - RNA, 11 nt, Human immunodeficiency virus
aagguagguca

SEQ ID NO: 17 - RNA, 11 nt, Human immunodeficiency virus
cuagacuagag

SEQ ID NO: 18 - RNA, 11 nt, Human immunodeficiency virus
gcaguaaguag

SEQ ID NO: 19 - DNA, 77 bp, Human immunodeficiency virus
ataacaaaagccttaggcatctcctatggcaggaagaagagagttaggcagggatattcaccattatcgtttcagcc

SEQ ID NO: 20 - protein, 36 aa, Artificial Sequence
  Fuzeon
YTSLIHSLIEESQNQQEKNEQELLELDKWASLWNWF